SEQUENCE LISTING (24 sequences)

SEQ ID NO: 1 - DNA, 2886 bp, Mus musculus
  CDS (24)..(2165)
gaattcggcacgagggccacaacatggctgcggcgccggggctgctcttctggctgttcgtgctgggggcgctctggtgggtcccgggccagtcggatctcagccacggacggcgtttctcggacctcaaagtgtgcggggacgaagagtgcagcatgttaatgtaccgtgggaaagctcttgaagacttcacgggccctgattgtcgttttgtgaattttaaaaaaggtgacgatgtatatgtctactacaaactggcagggggatcccttgaactttgggctggaagtgttgaacacagttttggatattttccaaaagatttgatcaaggtacttcataaatacacggaagaagagctacatattccagcagatgagacagactttgtctgctttgaaggaggaagagatgattttaatagttataatgtagaagagcttttaggatctttggaactggaggactctgtacctgaagagtcgaagaaagctgaagaagtttctcagcacagagagaaatctcctgaggagtctcgggggcgtgaacttgaccctgtgcctgagcccgaggcattcagagctgattcagaggatggagaaggtgctttctcagagagcaccgaggggctgcagggacagccctcagctcaggagagccaccctcacaccagcggtcctgcggctaacgctcagggagtgcagtcttcgttggacacttttgaagaaattctgcacgataaattgaaagtgccgggaagcgaaagcagaactggcaatagttctcctgcctcggtggagcgggagaagacagatgcttacaaagtcctgaaaacagaaatgagtcttgacctgaaaaccaagtttggctcaactgccgacgcccttgtgtctgacgacgaggcaaccagactggtcacttcattagaagacggcttcgatgaagcgctggatgctgagtattacccgatggaggaggaggaggaggtagaggaggatgcagacagctctgatgagctaccattactaaccttctcagacaaagatgagaaagtccccgggaagcccatgatagagaaatatttaacagataaggatccgaatttgagtgaagaggataaagttgaaccccctacctggggggatgcttttttttctattgtcacaggaggtgaagggaaaccaggtgtggtggatttagaaagatctatcgaggaggaggaagatgtgtcagtttcatctagccatcagaggaagccacagccagcagctggctatacagacagtgaggatgaaggtgacgatctttttgtagaagagcccaaaacaaatgatgtaaaggactcagaaacagacccagaacttgtcattacaggagaagaaaaggacatccaggaatccaggaaaggcctggtacagcctgagtcacaatcagaggatgccaagtcagaaacggcgagcgcttatcgtcttcagggtagcaagctcaaccctctgtcagctgctgaaaagggcagagactttacactaaaagccgtctttgaaaaaaaagaaaactcgagtggcctaaaagaatcagttatccatatctcaaaggaaacactacatgaagacaagaccagagagatccagagagacagcttggagagtgaattggtacacagagctttagggagttcagtgacagaaaataacaagcccaaatccttgggtgttgcaccactcttgggaaataacaagcccgatgcatccaaagacagtacagaggtcccagacggttccgtcagtggaccaaaagcaggccagcaggagggatttctagaaccaggtcttaaaactcagcaccaacctagattctcccctccagaggaaactggtccctcaagagaactggggggaaaagttcctatttcgggaagaaatctttcttggcagcaagagcaagacgtagctgctgtagtcggtaagcatgcgaatgagaagacagggtttccggaggaagagagcagggaagacgggacagatgccaactcaccaccctttggaccgttctgtggcatcgacactgtggtagcaccctttcatgcttcatcaaatcgggtcttcataagattccatgcggagtatgcaaccgtttcttcaggatttgaaataatgtggagtagctgaatcgggagagctgtgtgtaacccctctggctctgccccgcgccgtcagacgcaacccgcaaccctgcccagaactctgtggttctcatccccatctctgcccacgccatcaaacacggactgagcttctatggcctttacaagagaaagtgtatgaccaatttttttacatttggtgttctgaaggcagagcctggtggtgttccactcaggtgtacatgccatccttactggatgcaccatttagtggagaccttttaaaacgttctttaaagggtagaaattatcctggcacactgatgtacatttctaaaagttggtgatacaaatagctgtatggtttccactatacaaaagtcaaagtacgggtaataaacatttcttaaaaccataaaaaaaaaaaaaaaaaacggcacgaggagggggggcccggtacccaattcgccctatagtgagtcgtattacaattcactggccgtcgttttacaaccgtcgtgactgggaaaaccctggcgttacccaacttaatcgccttgcagcacatccccctttcgccagctggcgtaatagcgaagaggcccgcaccgatcgcccttccaacagttgcgcagcctgaatggcgaatggcaaattgtaagcgttaatattttgttaaaattcgcgttaaatttttgttaaatcagctcattttttaaccaataggccgaaatcggcaaaat

SEQ ID NO: 2 - DNA, 2142 bp, Mus musculus
  CDS (1)..(2142)
atggctgcggcgccggggctgctcttctggctgttcgtgctgggggcgctctggtgggtcccgggccagtcggatctcagccacggacggcgtttctcggacctcaaagtgtgcggggacgaagagtgcagcatgttaatgtaccgtgggaaagctcttgaagacttcacgggccctgattgtcgttttgtgaattttaaaaaaggtgacgatgtatatgtctactacaaactggcagggggatcccttgaactttgggctggaagtgttgaacacagttttggatattttccaaaagatttgatcaaggtacttcataaatacacggaagaagagctacatattccagcagatgagacagactttgtctgctttgaaggaggaagagatgattttaatagttataatgtagaagagcttttaggatctttggaactggaggactctgtacctgaagagtcgaagaaagctgaagaagtttctcagcacagagagaaatctcctgaggagtctcgggggcgtgaacttgaccctgtgcctgagcccgaggcattcagagctgattcagaggatggagaaggtgctttctcagagagcaccgaggggctgcagggacagccctcagctcaggagagccaccctcacaccagcggtcctgcggctaacgctcagggagtgcagtcttcgttggacacttttgaagaaattctgcacgataaattgaaagtgccgggaagcgaaagcagaactggcaatagttctcctgcctcggtggagcgggagaagacagatgcttacaaagtcctgaaaacagaaatgagtcttgacctgaaaaccaagtttggctcaactgccgacgcccttgtgtctgacgacgaggcaaccagactggtcacttcattagaagacggcttcgatgaagcgctggatgctgagtattacccgatggaggaggaggaggaggtagaggaggatgcagacagctctgatgagctaccattactaaccttctcagacaaagatgagaaagtccccgggaagcccatgatagagaaatatttaacagataaggatccgaatttgagtgaagaggataaagttgaaccccctacctggggggatgcttttttttctattgtcacaggaggtgaagggaaaccaggtgtggtggatttagaaagatctatcgaggaggaggaagatgtgtcagtttcatctagccatcagaggaagccacagccagcagctggctatacagacagtgaggatgaaggtgacgatctttttgtagaagagcccaaaacaaatgatgtaaaggactcagaaacagacccagaacttgtcattacaggagaagaaaaggacatccaggaatccaggaaaggcctggtacagcctgagtcacaatcagaggatgccaagtcagaaacggcgagcgcttatcgtcttcagggtagcaagctcaaccctctgtcagctgctgaaaagggcagagactttacactaaaagccgtctttgaaaaaaaagaaaactcgagtggcctaaaagaatcagttatccatatctcaaaggaaacactacatgaagacaagaccagagagatccagagagacagcttggagagtgaattggtacacagagctttagggagttcagtgacagaaaataacaagcccaaatccttgggtgttgcaccactcttgggaaataacaagcccgatgcatccaaagacagtacagaggtcccagacggttccgtcagtggaccaaaagcaggccagcaggagggatttctagaaccaggtcttaaaactcagcaccaacctagattctcccctccagaggaaactggtccctcaagagaactggggggaaaagttcctatttcgggaagaaatctttcttggcagcaagagcaagacgtagctgctgtagtcggtaagcatgcgaatgagaagacagggtttccggaggaagagagcagggaagacgggacagatgccaactcaccaccctttggaccgttctgtggcatcgacactgtggtagcaccctttcatgcttcatcaaatcgggtcttcataagattccatgcggagtatgcaaccgtttcttcaggatttgaaataatgtggagtagc

SEQ ID NO: 3 - protein, 714 aa, Mus musculus
MAAAPGLLFWLFVLGALWWVPGQSDLSHGRRFSDLKVCGDEECSMLMYRGKALEDFTGPDCRFVNFKKGDDVYVYYKLAGGSLELWAGSVEHSFGYFPKDLIKVLHKYTEEELHIPADETDFVCFEGGRDDFNSYNVEELLGSLELEDSVPEESKKAEEVSQHREKSPEESRGRELDPVPEPEAFRADSEDGEGAFSESTEGLQGQPSAQESHPHTSGPAANAQGVQSSLDTFEEILHDKLKVPGSESRTGNSSPASVEREKTDAYKVLKTEMSLDLKTKFGSTADALVSDDEATRLVTSLEDGFDEALDAEYYPMEEEEEVEEDADSSDELPLLTFSDKDEKVPGKPMIEKYLTDKDPNLSEEDKVEPPTWGDAFFSIVTGGEGKPGVVDLERSIEEEEDVSVSSSHQRKPQPAAGYTDSEDEGDDLFVEEPKTNDVKDSETDPELVITGEEKDIQESRKGLVQPESQSEDAKSETASAYRLQGSKLNPLSAAEKGRDFTLKAVFEKKENSSGLKESVIHISKETLHEDKTREIQRDSLESELVHRALGSSVTENNKPKSLGVAPLLGNNKPDASKDSTEVPDGSVSGPKAGQQEGFLEPGLKTQHQPRFSPPEETGPSRELGGKVPISGRNLSWQQEQDVAAVVGKHANEKTGFPEEESREDGTDANSPPFGPFCGIDTVVAPFHASSNRVFIRFHAEYATVSSGFEIMWSS

SEQ ID NO: 4 - protein, 690 aa, Mus musculus
DLSHGRRFSDLKVCGDEECSMLMYRGKALEDFTGPDCRFVNFKKGDDVYVYYKLAGGSLELWAGSVEHSFGYFPKDLIKVLHKYTEEELHIPADETDFVCFEGGRDDFNSYNVEELLGSLELEDSVPEESKKAEEVSQHREKSPEESRGRELDPVPEPEAFRADSEDGEGAFSESTEGLQGQPSAQESHPHTSGPAANAQGVQSSLDTFEEILHDKLKVPGSESRTGNSSPASVEREKTDAYKVLKTEMSLDLKTKFGSTADALVSDDEATRLVTSLEDGFDEALDAEYYPMEEEEEVEEDADSSDELPLLTFSDKDEKVPGKPMIEKYLTDKDPNLSEEDKVEPPTWGDAFFSIVTGGEGKPGVVDLERSIEEEEDVSVSSSHQRKPQPAAGYTDSEDEGDDLFVEEPKTNDVKDSETDPELVITGEEKDIQESRKGLVQPESQSEDAKSETASAYRLQGSKLNPLSAAEKGRDFTLKAVFEKKENSSGLKESVIHISKETLHEDKTREIQRDSLESELVHRALGSSVTENNKPKSLGVAPLLGNNKPDASKDSTEVPDGSVSGPKAGQQEGFLEPGLKTQHQPRFSPPEETGPSRELGGKVPISGRNLSWQQEQDVAAVVGKHANEKTGFPEEESREDGTDANSPPFGPFCGIDTVVAPFHASSNRVFIRFHAEYATVSSGFEIMWSS

SEQ ID NO: 5 - protein, 24 aa, Mus musculus
MAAAPGLLFWLFVLGALWWVPGQS

SEQ ID NO: 6 - protein, 125 aa, Mus musculus
MAAAPGLLFWLFVLGALWWVPGQSDLSHGRRFSDLKVCGDEECSMLMYRGKALEDFTGPDCRFVNFKKGDDVYVYYKLAGGSLELWAGSVEHSFGYFPKDLIKVLHKYTEEELHIPADETDFVCF

SEQ ID NO: 7 - DNA, 1263 bp, Homo sapiens
  CDS (34)..(1263)
gtcgacccacgcgtccgcccacgcgtccgcaacatggctgcggcgcctgggctgctcgtctggctgctcgtgctccggctgccctggcgggtgccgggccagctggaccccagcactggccggcggttctcggagcacaaactctgcgcggacgacgaatgcagcatgttaatgtaccgcggtgaggctcttgaagatttcacaggcccggattgtcgttttgtgaattttaaaaaaggtgatcctgtatatgtttactataaactggcaagaggatggcctgaagtttgggctggaagtgttggacgcacttttggatattttccaaaagatttaatccaggtagttcatgaatataccaaagaagagctacaagttccaacagatgagacggattttgtttgttttgatggaggaagagatgattttcataattataatgtagaagaacttttagggtttttggaactgtacaattctgcagctacagattctgagaaagctgtagaaaaaactttacaggatatggaaaaaaaccctgaattatctaaggaaagggaacctgaacctgaaccagtagaagccaactcagaggaaagtgatagtgtattctcagaaaacactgaggatcttcaggaacagtttacaactcagaagcaccactcccatgcaaacagccaagcaaatcatgctcagggagagcaggcttcatttgaatcttttgaagaaatgctgcaagataaactaaaagtgccagaaagtgaaaacaacaaaaccagcaatagttctcaggtctcaaatgaacaggataagattgatgcctataaacttttgaaaaaagaaatgactctagacttgaaaaccaaatttggctcaacagctgatgcacttgtatctgatgatgagacaaccagactcgttacttcattagaagatgattttgatgaggaattggatactgagtattatgcagttggaaaggaagatgaggagaaccaagaagactttgatgagttgccattacttacctttacagatggggaagatatgaaaactccagcaaagtctggcgttgagaaatatccaacagataaagagcagaattcaaatgaagaggacaaggttcagctaactgtgccccctggcatcaaaaatgatgataaaaatatactaacaacctggggggacactatcttctctattgtcacaggaggtgaagaaacaagagatacgatggatttagagagctctagttcagaggaa

SEQ ID NO: 8 - DNA, 1230 bp, Homo sapiens
  CDS (1)..(1230)
atggctgcggcgcctgggctgctcgtctggctgctcgtgctccggctgccctggcgggtgccgggccagctggaccccagcactggccggcggttctcggagcacaaactctgcgcggacgacgaatgcagcatgttaatgtaccgcggtgaggctcttgaagatttcacaggcccggattgtcgttttgtgaattttaaaaaaggtgatcctgtatatgtttactataaactggcaagaggatggcctgaagtttgggctggaagtgttggacgcacttttggatattttccaaaagatttaatccaggtagttcatgaatataccaaagaagagctacaagttccaacagatgagacggattttgtttgttttgatggaggaagagatgattttcataattataatgtagaagaacttttagggtttttggaactgtacaattctgcagctacagattctgagaaagctgtagaaaaaactttacaggatatggaaaaaaaccctgaattatctaaggaaagggaacctgaacctgaaccagtagaagccaactcagaggaaagtgatagtgtattctcagaaaacactgaggatcttcaggaacagtttacaactcagaagcaccactcccatgcaaacagccaagcaaatcatgctcagggagagcaggcttcatttgaatcttttgaagaaatgctgcaagataaactaaaagtgccagaaagtgaaaacaacaaaaccagcaatagttctcaggtctcaaatgaacaggataagattgatgcctataaacttttgaaaaaagaaatgactctagacttgaaaaccaaatttggctcaacagctgatgcacttgtatctgatgatgagacaaccagactcgttacttcattagaagatgattttgatgaggaattggatactgagtattatgcagttggaaaggaagatgaggagaaccaagaagactttgatgagttgccattacttacctttacagatggggaagatatgaaaactccagcaaagtctggcgttgagaaatatccaacagataaagagcagaattcaaatgaagaggacaaggttcagctaactgtgccccctggcatcaaaaatgatgataaaaatatactaacaacctggggggacactatcttctctattgtcacaggaggtgaagaaacaagagatacgatggatttagagagctctagttcagaggaa

SEQ ID NO: 9 - protein, 410 aa, Homo sapiens
MAAAPGLLVWLLVLRLPWRVPGQLDPSTGRRFSEHKLCADDECSMLMYRGEALEDFTGPDCRFVNFKKGDPVYVYYKLARGWPEVWAGSVGRTFGYFPKDLIQVVHEYTKEELQVPTDETDFVCFDGGRDDFHNYNVEELLGFLELYNSAATDSEKAVEKTLQDMEKNPELSKEREPEPEPVEANSEESDSVFSENTEDLQEQFTTQKHHSHANSQANHAQGEQASFESFEEMLQDKLKVPESENNKTSNSSQVSNEQDKIDAYKLLKKEMTLDLKTKFGSTADALVSDDETTRLVTSLEDDFDEELDTEYYAVGKEDEENQEDFDELPLLTFTDGEDMKTPAKSGVEKYPTDKEQNSNEEDKVQLTVPPGIKNDDKNILTTWGDTIFSIVTGGEETRDTMDLESSSSEE

SEQ ID NO: 10 - protein, 387 aa, Homo sapiens
LDPSTGRRFSEHKLCADDECSMLMYRGEALEDFTGPDCRFVNFKKGDPVYVYYKLARGWPEVWAGSVGRTFGYFPKDLIQVVHEYTKEELQVPTDETDFVCFDGGRDDFHNYNVEELLGFLELYNSAATDSEKAVEKTLQDMEKNPELSKEREPEPEPVEANSEESDSVFSENTEDLQEQFTTQKHHSHANSQANHAQGEQASFESFEEMLQDKLKVPESENNKTSNSSQVSNEQDKIDAYKLLKKEMTLDLKTKFGSTADALVSDDETTRLVTSLEDDFDEELDTEYYAVGKEDEENQEDFDELPLLTFTDGEDMKTPAKSGVEKYPTDKEQNSNEEDKVQLTVPPGIKNDDKNILTTWGDTIFSIVTGGEETRDTMDLESSSSEE

SEQ ID NO: 11 - protein, 23 aa, Homo sapiens
MAAAPGLLVWLLVLRLPWRVPGQ

SEQ ID NO: 12 - protein, 125 aa, Homo sapiens
MAAAPGLLVWLLVLRLPWRVPGQLDPSTGRRFSEHKLCADDECSMLMYRGEALEDFTGPDCRFVNFKKGDPVYVYYKLARGWPEVWAGSVGRTFGYFPKDLIQVVHEYTKEELQVPTDETDFVCF

SEQ ID NO: 13 - protein, 133 aa, Artificial Sequence
  consensus sequence
  VARIANT 2-7, 9-13, 15-35: Xaa = any anino acid
  VARIANT 37: Xaa = Leu, or Val
  VARIANT 39: Xaa = Ala, or Gly
  VARIANT 41, 45-51, 54, 56-58, 64-68, 70, 71, 75, 76, 79-85, 87,91-102, 107-125, 127, 128, 131, 133: Xaa = any amino acid
MXXXXXXLXXXXXLXXXXXXXXXXXXXXXXXXXXXKXCXDXECSXXXXXXXALXDXXXPDCRFXXXXXGXXVYVXXKLXXXXXXXWXGSVXXXXXXXXXXXXGYFPXXXXXXXXXXXXXXXXXXXDXXDFXCX

SEQ ID NO: 14 - DNA, 8121 bp, Homo sapiens
  5′UTR (1)...(4)
  CDS (5)...(5725)
  3′UTR (5726)...(8121)
caacatggctgcggcgcctgggctgctagtctggctgctcgtgctccggctgccctggcgggtgccgggccagctggaccccagcactggccggcggttctcggagcacaaactctgcgcggacgacgaatgcagcatgttaatgtaccgcggtgaggctcttgaagatttcacaggcccggattgtcgttttgtgaattttaaaaaaggtgatcctgtatatgtttactataaactggcaagaggatggcctgaagtttgggctggaagtgttggacgcacttttggatattttccaaaagatttaatccaggtagttcatgaatataccaaagaagagctacaagttccaacagatgagacggattttgtttgttttgatggaggaagagatgattttcataattataatgtagaagaacttttagggtttttggaactgtacaattctgcagctacagattctgagaaagctgtagaaaaaactttacaggatatggaaaaaaaccctgaattatctaaggaaagggaacctgaacctgaaccagtagaagccaactcagaggaaagtgatagtgtattctcagaaaacactgaggatcttcaggaacagtttacaactcagaagcaccactcccatgcaaacagccaagcaaatcatgctcagggagagcaggcttcatttgaatcttttgaagaaatgctgcaagataaactaaaagtgccagaaagtgaaaacaacaaaaccagcaatagttctcaggtctcaaatgaacaggataagattgatgcctataaacttttgaaaaaagaaatgactctagacttgaaaaccaaatttggctcaacagctgatgcacttgtatctgatgatgagacaaccagactcgttacttcattagaagatgattttgatgaggaattggatactgagtattatgcagttggaaaggaagatgaggagaaccaagaagactttgatgagttgccattacttacctttacagatggggaagatatgaaaactccagcaaagtctggcgttgagaaatatccaacagataaagagcagaattcaaatgaagaggacaaggttcagctaactgtgccccctggcatcaaaaatgatgataaaaatatactaacaacctggggggacactatcttctctattgtcacaggaggtgaagaaacaagagatacgatggatttagagagctctagttcagaggaagaaaaagaagatgatgatgatgcattagtcccagatagcaaacaggggaaaccacagtcagcaacagattatagtgaccctgacaatgtagatgatggtctttttattgtagacattcctaaaacaaataatgacaaagaagtaaacgcagaacatcacattaaaggaaaagggaggggagttcaggaatccaagaggggcctggtacaagatgagacagaattagaggatgaaaatcaagaaggcatgactgtgcacagttctgttcacagcaataacctcaactctatgccagctgctgaaaagggtaaagacacattaaaatcagcttatgatgatacagaaaatgacctaaaaggagcagctattcatatctcaaaaggaatgctccacgaagaaaagcctggagagcagattttggaaggtggctcagagagtgaatctgcacagaaagctgcagggaatcaaatgaatgacagaaagattcaacaggaatccctgggtagtgcaccactcatgggagatgaccaccctaacgcatccagagacagtgtggagggagacgctttggtaaatggggccaaactgcacacgctttcagtggagcatcaacgtgaggaattgaaagaggaattagttcttaaaactcaaaaccaacctagattctcctctccagatgagattgatttgcccagagaactggaagacgaggttcccattctgggaagaaatcttccctggcaacaagaaagagatgtggctgccacagccagtaagcaaatgagtgagaagataaggctctctgagggagaagccaaagaggactccttggatgaagagttttttcatcacaaggcaatgcagggcacagaggtaggacagacagaccaaactgacagcacaggaggaccagctttcctttctaaagtagaagaggatgattatccctctgaagaactactagaggatgaaaacgctataaatgcaaaacggtctaaagaaaaaaacccggggaatcagggcaggcagtttgatgttaatctgcaagtccctgacagagcagttttagggaccattcatccagatccagaaattgaagaaagcaagcaagaaactagtatgattttggatagtgaaaaaacaagtgagactgctgccaaaggggtcaacacaggaggcagggaaccaaatacaatggtggaaaaagaacgccctctggcagataagaaagcacagagaccatttgaacgaagtgacttttctgacagcataaaaattcagactccagaattaggtgaagtgtttcagaataaagattctgattatctgaagaacgacaaccctgaggaacatctgaagacctcagggcttgcaggggagcctgagggagaactctcaaaagaggaccatgagaacacagagaagtacatgggcacagaaagccaggggtctgctgctgcagaacctgaagatgactcgttccactggactccacatacaagtgtagagccagggcatagtgacaagagggaggacttacttatcataagcagcttctttaaagaacaacagtctttgcagcggttccagaagtactttaatgtccatgagctggaagccttgctacaagaaatgtcatcaaaactgaagtcagcgcagcaggagagcctgccctataatatggaaaaagtcctagataaggtcttccgtgcttctgagtcacaaattctgagcatagcagaaaaaatgcttgatactcgtgtggctgaaaatagagatctgggaatgaacgaaaataacatatttgaagaggctgcagtgcttgatgacattcaagacctcatctattttgtcaggtacaagcactccacagcagaggagacagccacactggtgatggcaccacctctagaggaaggcttgggtggagcaatggaagagatgcaaccactgcatgaagataatttctcacgagagaagacagcagaacttaatgtgcaggttcctgaagaacccacccacttggaccaacgtgtgattggggacactcatgcctcagaagtgtcacagaagccaaatactgagaaagacctggacccagggccagttacaacagaagacactcctatggatgctattgatgcaaacaagcaaccagagacagccgccgaagagccggcaagtgtcacacctttggaaaacgcaatccttctaatatattcattcatgttttatttaactaagtcgctagttgctacattgcctgatgatgttcagcctgggcctgatttttatggactgccatggaaacctgtatttatcactgccttcttgggaattgcttcgtttgccattttcttatggagaactgtccttgttgtgaaggatagagtatatcaagtcacggaacagcaaatttctgagaagttgaagactatcatgaaagaaaatacagaacttgtacaaaaattgtcaaattatgaacagaagatcaaggaatcaaagaaacatgttcaggaaaccaggaaacaaaatatgattctctctgatgaagcaattaaatataaggataaaatcaagacacttgaaaaaaatcaggaaattctggatgacacagctaaaaatcttcgtgttatgctagaatctgagagagaacagaatgtcaagaatcaggacttgatatcagaaaacaagaaatctatagagaagttaaaggatgttatttcaatgaatgcctcagaattttcagaggttcagattgcacttaatgaagctaagcttagtgaagagaaggtgaagtctgaatgccatcgggttcaagaagaaaatgctaggcttaagaagaaaaaagagcagttgcagcaggaaatcgaagactggagtaaattacatgctgagctcagtgagcaaatcaaatcatttgagaagtctcagaaagatttggaagtagctcttactcacaaggatgataatattaatgctttgactaactgcattacacagttgaatctgttagagtgtgaatctgaatctgagggtcaaaataaaggtggaaatgattcagatgaattagcaaatggagaagtgggaggtgaccggaatgagaagatgaaaaatcaaattaagcagatgatggatgtctctcggacacagactgcaatatcggtagttgaagaggatctaaagcttttacagcttaagctaagagcctccgtgtccactaaatgtaacctggaagaccaggtaaagaaattggaagatgaccgcaactcactacaagctgccaaagctggactggaagatgaatgcaaaaccttgaggcagaaagtggagattctgaatgagctctatcagcagaaggagatggctttgcaaaagaaactgagtcaagaagagtatgaacggcaagaaagagagcacaggctgtcagctgcagatgaaaaggcagtttcggctgcagaggaagtaaaaacttacaagcggagaattgaagaaatggaggatgaattacagaagacagagcggtcatttaaaaaccagatcgctacccatgagaagaaagctcatgaaaactggctcaaagctcgtgctgcagaaagagctatagctgaagagaaaagggaagctgccaatttgagacacaaattattagaattaacacaaaagatggcaatgctgcaagaagaacctgtgattgtaaaaccaatgccaggaaaaccaaatacacaaaaccctccacggagaggtcctctgagccagaatggctcttttggcccatcccctgtgagtggtggagaatgctcccctccattgacagtggagccacccgtgagacctctctctgctactctcaatcgaagagatatgcctagaagtgaatttggatcagtggacgggcctctacctcatcctcgatggtcagctgaggcatctgggaaaccctctccttctgatccaggatctggtacagctaccatgatgaacagcagctcaagaggctcttcccctaccagggtactcgatgaaggcaaggttaatatggctccaaaagggccccctcctttcccaggagtccctctcatgagcacccccatgggaggccctgtaccaccacccattcgatatggaccaccacctcagctctgcggaccttttgggcctcggccacttcctccaccctttggccctggtatgcgtccaccactaggcttaagagaatttgcaccaggcgttccaccaggaagacgggacctgcctctccaccctcggggatttttacctggacacgcaccatttagacctttaggttcacttggcccaagagagtactttattcctggtacccgattaccacccccaacccatggtccccaggaatacccaccaccacctgctgtaagagacttactgccgtcaggctctagagatgagcctccacctgcctctcagagcactagccaggactgttcacaggctttaaaacagagcccataaaactatgacctctgaggtttcattggaaagaaagtgtactgtgcattatccattacagtaaaggatttcattggcttcaaaatccaaaagtttattttaaaaggtttgttgttagaactaagctgccttggcagtgtgcatttttgagccaaacaattcaaaaatgtcatttcttccctaaataaaaatcaccttttaagctagagcgtccttacaactttgaaatgtgcaataaagaatacctgtgttttagctaatgtagcatatgtaattgcaaaatgatttagaatgtcatgaaaaatatgaacatttcctgtggaaatgctttaagaacatgtatttccattatcctatttttagtgtacaccagctgaatacggagcaatggtgtttataagcgtttttttaaactatctggtcacaaagactgttacgctaaaaatgtttactaaaagatcactaaactatctcccctcttgctgaagttctttgtagtaatagctcataaaaatttgtttattaatatttcccaagtgtctgttgactcattggactgttatgaggcttgtgccatttggggaacatgtaaactcaggctcccagaactgaagatggtggctggtggcacacttccggctgctcctccgtcacctgtgaactctacaagtgatgtctttttatttcaaagaagtttatttcccacttgtatagcattcacatgctttctttacgatcctcattgtctatttgagaatggttttctgagagtgagtttacattagtagcaagagttgtttgacctgatgttccattgtttttaccattcctgtagaaaaagggtgcacaacagaaaaatgaaaatgatgtgtcatggccataaaagtatagaaatctttaaaaattttaaaatgtacagtcccttatctatctttcccattccttgccactgatttttgaggaatataataaaaagattggaagagtataatgccatgagaaagaatgatttaggactgtgagggttataacatgccctaggtcagcaaccaagggttgaaatcagttctgttttagggggaaatggggggggcgacagatattattccaaaattaatattaattaatatttaaacgttggtgtttttatttaaaaatcagtaactaaccatctggaattgcaccatacttaaagtcttatccattactacactgtctttaaaacaatgtttctttaaatactctacaacgtttctaagaacgaacttcagacattttaattacagtaataatagcactccttttaaggagtttcagatccacactaaaactaaaatcataaaaggctgatacttttgtttgctgctaggctatattcttccattctttgaagtcctatgatgtaatatttttgaaacctagtgtatgtcttgtcactgttgtgatatttaatcgattaagaataccttgtaaaaaggagcaaaagcttcaatgtgaaacaattttctctctttatactaaacaactgaagatagatagtttagaaagataaggacctttgaaagaagacaactctgtcaaagttcataaggaatataaaaattcttcaggaaaagagaattcaatctatatgtcctcccgtttaatatcaagaatagaagaaattaagaggaaaactccacagaagagcataggccacttttagccatgtaaaaataagattaagtcacaaatacaacttttgaatttacctgtcaatatctctttaggacacaaaacaatgctgaagttaatataatttctaattttaaatgtcatttaagtgtagattatgccatctaggaaggtaagtaggaaaggtaaattaaatctatttttaaaattcaaaatattagagtatttttcccctctaaagccttttttggtgattattctgtatctgacataattgagaaactggtaagctgtaaagattccagtgtagcttctctgagaagttgtgagccagtccataactgcttcctcacatccatctgattgcaccatttctgcagcaaaccccaaagcagggtgccaatatgcagatggcatagggagtatcatccctcagccaaatcacttttccatctctaaagtttcatctattttggaagtcatctccaactaattgtgtctggatttagttgctaaaattgtcttatttatttatgaagcagcaatattcagcctgaaagcatttctgccatagttgttgtagttatatcgccaatggctgatttttttcattggaaagtaaatttaagtaattcgtgggatgtggtatattctgtgtcaacttcaagataatcactcattttctcgttatattcaggtctgaattaaagttaagttaatca

SEQ ID NO: 15 - DNA, 5721 bp, Homo sapiens
  CDS (1)...(5721)
atggctgcggcgcctgggctgctagtctggctgctcgtgctccggctgccctggcgggtgccgggccagctggaccccagcactggccggcggttctcggagcacaaactctgcgcggacgacgaatgcagcatgttaatgtaccgcggtgaggctcttgaagatttcacaggcccggattgtcgttttgtgaattttaaaaaaggtgatcctgtatatgtttactataaactggcaagaggatggcctgaagtttgggctggaagtgttggacgcacttttggatattttccaaaagatttaatccaggtagttcatgaatataccaaagaagagctacaagttccaacagatgagacggattttgtttgttttgatggaggaagagatgattttcataattataatgtagaagaacttttagggtttttggaactgtacaattctgcagctacagattctgagaaagctgtagaaaaaactttacaggatatggaaaaaaaccctgaattatctaaggaaagggaacctgaacctgaaccagtagaagccaactcagaggaaagtgatagtgtattctcagaaaacactgaggatcttcaggaacagtttacaactcagaagcaccactcccatgcaaacagccaagcaaatcatgctcagggagagcaggcttcatttgaatcttttgaagaaatgctgcaagataaactaaaagtgccagaaagtgaaaacaacaaaaccagcaatagttctcaggtctcaaatgaacaggataagattgatgcctataaacttttgaaaaaagaaatgactctagacttgaaaaccaaatttggctcaacagctgatgcacttgtatctgatgatgagacaaccagactcgttacttcattagaagatgattttgatgaggaattggatactgagtattatgcagttggaaaggaagatgaggagaaccaagaagactttgatgagttgccattacttacctttacagatggggaagatatgaaaactccagcaaagtctggcgttgagaaatatccaacagataaagagcagaattcaaatgaagaggacaaggttcagctaactgtgccccctggcatcaaaaatgatgataaaaatatactaacaacctggggggacactatcttctctattgtcacaggaggtgaagaaacaagagatacgatggatttagagagctctagttcagaggaagaaaaagaagatgatgatgatgcattagtcccagatagcaaacaggggaaaccacagtcagcaacagattatagtgaccctgacaatgtagatgatggtctttttattgtagacattcctaaaacaaataatgacaaagaagtaaacgcagaacatcacattaaaggaaaagggaggggagttcaggaatccaagaggggcctggtacaagatgagacagaattagaggatgaaaatcaagaaggcatgactgtgcacagttctgttcacagcaataacctcaactctatgccagctgctgaaaagggtaaagacacattaaaatcagcttatgatgatacagaaaatgacctaaaaggagcagctattcatatctcaaaaggaatgctccacgaagaaaagcctggagagcagattttggaaggtggctcagagagtgaatctgcacagaaagctgcagggaatcaaatgaatgacagaaagattcaacaggaatccctgggtagtgcaccactcatgggagatgaccaccctaacgcatccagagacagtgtggagggagacgctttggtaaatggggccaaactgcacacgctttcagtggagcatcaacgtgaggaattgaaagaggaattagttcttaaaactcaaaaccaacctagattctcctctccagatgagattgatttgcccagagaactggaagacgaggttcccattctgggaagaaatcttccctggcaacaagaaagagatgtggctgccacagccagtaagcaaatgagtgagaagataaggctctctgagggagaagccaaagaggactccttggatgaagagttttttcatcacaaggcaatgcagggcacagaggtaggacagacagaccaaactgacagcacaggaggaccagctttcctttctaaagtagaagaggatgattatccctctgaagaactactagaggatgaaaacgctataaatgcaaaacggtctaaagaaaaaaacccggggaatcagggcaggcagtttgatgttaatctgcaagtccctgacagagcagttttagggaccattcatccagatccagaaattgaagaaagcaagcaagaaactagtatgattttggatagtgaaaaaacaagtgagactgctgccaaaggggtcaacacaggaggcagggaaccaaatacaatggtggaaaaagaacgccctctggcagataagaaagcacagagaccatttgaacgaagtgacttttctgacagcataaaaattcagactccagaattaggtgaagtgtttcagaataaagattctgattatctgaagaacgacaaccctgaggaacatctgaagacctcagggcttgcaggggagcctgagggagaactctcaaaagaggaccatgagaacacagagaagtacatgggcacagaaagccaggggtctgctgctgcagaacctgaagatgactcgttccactggactccacatacaagtgtagagccagggcatagtgacaagagggaggacttacttatcataagcagcttctttaaagaacaacagtctttgcagcggttccagaagtactttaatgtccatgagctggaagccttgctacaagaaatgtcatcaaaactgaagtcagcgcagcaggagagcctgccctataatatggaaaaagtcctagataaggtcttccgtgcttctgagtcacaaattctgagcatagcagaaaaaatgcttgatactcgtgtggctgaaaatagagatctgggaatgaacgaaaataacatatttgaagaggctgcagtgcttgatgacattcaagacctcatctattttgtcaggtacaagcactccacagcagaggagacagccacactggtgatggcaccacctctagaggaaggcttgggtggagcaatggaagagatgcaaccactgcatgaagataatttctcacgagagaagacagcagaacttaatgtgcaggttcctgaagaacccacccacttggaccaacgtgtgattggggacactcatgcctcagaagtgtcacagaagccaaatactgagaaagacctggacccagggccagttacaacagaagacactcctatggatgctattgatgcaaacaagcaaccagagacagccgccgaagagccggcaagtgtcacacctttggaaaacgcaatccttctaatatattcattcatgttttatttaactaagtcgctagttgctacattgcctgatgatgttcagcctgggcctgatttttatggactgccatggaaacctgtatttatcactgccttcttgggaattgcttcgtttgccattttcttatggagaactgtccttgttgtgaaggatagagtatatcaagtcacggaacagcaaatttctgagaagttgaagactatcatgaaagaaaatacagaacttgtacaaaaattgtcaaattatgaacagaagatcaaggaatcaaagaaacatgttcaggaaaccaggaaacaaaatatgattctctctgatgaagcaattaaatataaggataaaatcaagacacttgaaaaaaatcaggaaattctggatgacacagctaaaaatcttcgtgttatgctagaatctgagagagaacagaatgtcaagaatcaggacttgatatcagaaaacaagaaatctatagagaagttaaaggatgttatttcaatgaatgcctcagaattttcagaggttcagattgcacttaatgaagctaagcttagtgaagagaaggtgaagtctgaatgccatcgggttcaagaagaaaatgctaggcttaagaagaaaaaagagcagttgcagcaggaaatcgaagactggagtaaattacatgctgagctcagtgagcaaatcaaatcatttgagaagtctcagaaagatttggaagtagctcttactcacaaggatgataatattaatgctttgactaactgcattacacagttgaatctgttagagtgtgaatctgaatctgagggtcaaaataaaggtggaaatgattcagatgaattagcaaatggagaagtgggaggtgaccggaatgagaagatgaaaaatcaaattaagcagatgatggatgtctctcggacacagactgcaatatcggtagttgaagaggatctaaagcttttacagcttaagctaagagcctccgtgtccactaaatgtaacctggaagaccaggtaaagaaattggaagatgaccgcaactcactacaagctgccaaagctggactggaagatgaatgcaaaaccttgaggcagaaagtggagattctgaatgagctctatcagcagaaggagatggctttgcaaaagaaactgagtcaagaagagtatgaacggcaagaaagagagcacaggctgtcagctgcagatgaaaaggcagtttcggctgcagaggaagtaaaaacttacaagcggagaattgaagaaatggaggatgaattacagaagacagagcggtcatttaaaaaccagatcgctacccatgagaagaaagctcatgaaaactggctcaaagctcgtgctgcagaaagagctatagctgaagagaaaagggaagctgccaatttgagacacaaattattagaattaacacaaaagatggcaatgctgcaagaagaacctgtgattgtaaaaccaatgccaggaaaaccaaatacacaaaaccctccacggagaggtcctctgagccagaatggctcttttggcccatcccctgtgagtggtggagaatgctcccctccattgacagtggagccacccgtgagacctctctctgctactctcaatcgaagagatatgcctagaagtgaatttggatcagtggacgggcctctacctcatcctcgatggtcagctgaggcatctgggaaaccctctccttctgatccaggatctggtacagctaccatgatgaacagcagctcaagaggctcttcccctaccagggtactcgatgaaggcaaggttaatatggctccaaaagggccccctcctttcccaggagtccctctcatgagcacccccatgggaggccctgtaccaccacccattcgatatggaccaccacctcagctctgcggaccttttgggcctcggccacttcctccaccctttggccctggtatgcgtccaccactaggcttaagagaatttgcaccaggcgttccaccaggaagacgggacctgcctctccaccctcggggatttttacctggacacgcaccatttagacctttaggttcacttggcccaagagagtactttattcctggtacccgattaccacccccaacccatggtccccaggaatacccaccaccacctgctgtaagagacttactgccgtcaggctctagagatgagcctccacctgcctctcagagcactagccaggactgttcacaggctttaaaacagagccca

SEQ ID NO: 16 - protein, 1907 aa, Homo sapiens [sequence omitted]

SEQ ID NO: 17 - protein, 1884 aa, Homo sapiens
LDPSTGRRFSEHKLCADDECSMLMYRGEALEDFTGPDCRFVNFKKGDPVYVYYKLARGWPEVWAGSVGRTFGYFPKDLIQVVHEYTKEELQVPTDETDFVCFDGGRDDFHNYNVEELLGFLELYNSAATDSEKAVEKTLQDMEKNPELSKEREPEPEPVEANSEESDSVFSENTEDLQEQFTTQKHHSHANSQANHAQGEQASFESFEEMLQDKLKVPESENNKTSNSSQVSNEQDKIDAYKLLKKEMTLDLKTKFGSTADALVSDDETTRLVTSLEDDFDEELDTEYYAVGKEDEENQEDFDELPLLTFTDGEDMKTPAKSGVEKYPTDKEQNSNEEDKVQLTVPPGIKNDDKNILTTWGDTIFSIVTGGEETRDTMDLESSSSEEEKEDDDDALVPDSKQGKPQSATDYSDPDNVDDGLFIVDIPKTNNDKEVNAEHHIKGKGRGVQESKRGLVQDETELEDENQEGMTVHSSVHSNNLNSMPAAEKGKDTLKSAYDDTENDLKGAAIHISKGMLHEEKPGEQILEGGSESESAQKAAGNQMNDRKIQQESLGSAPLMGDDHPNASRDSVEGDALVNGAKLHTLSVEHQREELKEELVLKTQNQPRFSSPDEIDLPRELEDEVPILGRNLPWQQERDVAATASKQMSEKIRLSEGEAKEDSLDEEFFHHKAMQGTEVGQTDQTDSTGGPAFLSKVEEDDYPSEELLEDENAINAKRSKEKNPGNQGRQFDVNLQVPDRAVLGTIHPDPEIEESKQETSMILDSEKTSETAAKGVNTGGREPNTMVEKERPLADKKAQRPFERSDFSDSIKIQTPELGEVFQNKDSDYLKNDNPEEHLKTSGLAGEPEGELSKEDHENTEKYMGTESQGSAAAEPEDDSFHWTPHTSVEPGHSDKREDLLIISSFFKEQQSLQRFQKYFNVHELEALLQEMSSKLKSAQQESLPYNMEKVLDKVFRASESQILSIAEKMLDTRVAENRDLGMNENNIFEEAAVLDDIQDLIYFVRYKHSTAEETATLVMAPPLEEGLGGAMEEMQPLHEDNFSREKTAELNVQVPEEPTHLDQRVIGDTHASEVSQKPNTEKDLDPGPVTTEDTPMDAIDANKQPETAAEEPASVTPLENAILLIYSFMFYLTKSLVATLPDDVQPGPDFYGLPWKPVFITAFLGIASFAIFLWRTVLVVKDRVYQVTEQQISEKLKTIMKENTELVQKLSNYEQKIKESKKHVQETRKQNMILSDEAIKYKDKIKTLEKNQEILDDTAKNLRVMLESEREQNVKNQDLISENKKSIEKLKDVISMNASEFSEVQIALNEAKLSEEKVKSECHRVQEENARLKKKKEQLQQEIEDWSKLHAELSEQIKSFEKSQKDLEVALTHKDDNINALTNCITQLNLLECESESEGQNKGGNDSDELANGEVGGDRNEKMKNQIKQMMDVSRTQTAISVVEEDLKLLQLKLRASVSTKCNLEDQVKKLEDDRNSLQAAKAGLEDECKTLRQKVEILNELYQQKEMALQKKLSQEEYERQEREHRLSAADEKAVSAAEEVKTYKRRIEEMEDELQKTERSFKNQIATHEKKAHENWLKARAAERAIAEEKREAANLRHKLLELTQKMAMLQEEPVIVKPMPGKPNTQNPPRRGPLSQNGSFGPSPVSGGECSPPLTVEPPVRPLSATLNRRDMPRSEFGSVDGPLPHPRWSAEASGKPSPSDPGSGTATMMNSSSRGSSPTRVLDEGKVNMAPKGPPPFPGVPLMSTPMGGPVPPPIRYGPPPQLCGPFGPRPLPPPFGPGMRPPLGLREFAPGVPPGRRDLPLHPRGFLPGHAPFRPLGSLGPREYFIPGTRLPPPTHGPQEYPPPPAVRDLLPSGSRDEPPPASQSTSQDCSQALKQSP

SEQ ID NO: 18 - protein, 23 aa, Homo sapiens
MAAAPGLLVWLLVLRLPWRVPGQ

SEQ ID NO: 19 - protein, 125 aa, Homo sapiens; identical to SEQ ID NO: 12

SEQ ID NO: 20 - protein, 12 aa, Mus musculus
DLSHGRRFSDLK

SEQ ID NO: 21 - protein, 15 aa, Mus musculus
EDFTGPDCRFVNFKK

SEQ ID NO: 22 - protein, 14 aa, Homo sapiens
QLDPSTGRRFSEHK

SEQ ID NO: 23 - protein, 15 aa, Homo sapiens
EDFTGPDCRFVNFKK

SEQ ID NO: 24 - protein, 14 aa, Homo sapiens
GFLELYNSAATDSE

(19) United States
(12) Patent Application Publication
(10) Pub. No.: US 2007/0031439 A1
(43) Pub. Date: Feb. 8, 2007

(54) Novel protein related to melanoma-inhibiting protein and uses thereof

(75) Inventors: Yang Pan, Bellevue, WA (US); Thomas M. Barnes, Brookline, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 11/583,426

(22) Filed: Oct. 19, 2006

Related U.S. Application Data
(63) Continuation of application No. 10/409,966, filed on Apr. 9, 2003.
Application No. 10/409,966 is a continuation of application No. 09/785,770, filed on Feb. 16, 2001, now abandoned.
Application No. 09/785,770 is a continuation-in-part of application No. 09/387,462, filed on Sep. 1, 1999, now abandoned.
Application No. 09/387,462 is a continuation-in-part of application No. 09/145,056, filed on Sep. 1, 1998, now abandoned.

(51) Int. Cl. A61K 39/395; C12Q 1/68; G01N 33/574; C07H 21/04; C12P 21/06; C07K 14/82; C07K 16/30

(52) U.S. Cl. 424/155.1; 536/23.5; 530/350; 530/388.8; 435/6; 435/69.1; 435/7.23; 435/320.1; 435/325

(57) ABSTRACT

Novel TANGO 130 nucleic acid molecules which encode proteins having homology to melanoma-inhibiting protein are disclosed. In addition to TANGO 130 nucleic acid molecules and proteins, the invention further provides isolated TANGO 130 fusion proteins, antigenic peptides and anti-TANGO 130 antibodies. The invention also provides vectors containing nucleic acid molecules of the invention, host cells into which the vectors have been introduced and non-human transgenic animals in which a TANGO 130 gene has been introduced or disrupted. Diagnostic, screening and therapeutic methods utilizing compositions of the invention are also provided.

### CROSS-REFERENCE TO RELATED APPLICATIONS

This application is a continuation of U.S. application Ser. No. 09/785,770, filed on Feb. 16, 2001, which is a continuation-in-part of U.S. application Ser. No. 09/387,462, filed on Sep. 1, 1999, which is a continuation-in-part of U.S. application Ser. No. 09/145,056, filed on Sep. 1, 1998. The contents of each of the applications cross-referenced in this section are incorporated into this disclosure by this reference.

### BACKGROUND OF THE INVENTION

A variety of factors participate in the tightly controlled regulation of cell growth and differentiation. One molecule believed to be involved in such regulation is Melanoma-Inhibiting Protein (MIA). Human and murine MIA cDNAs were first cloned from malignant melanoma cells and shown to inhibit growth of melanoma cells in vitro (Blesch et al. (1994) Cancer Res. 54:5695). Human MIA cDNA encodes a 24 amino acid signal peptide and a mature 107 amino acid secreted protein, and shares little or no homology with other known proteins.

Cancer cells and embryonic cells are growth inhibited after treatment with MIA, observed as cell cycle arrest accompanied by a rounded up cell morphology and decreased adherence to the substrate. Furthermore, MIA expression is enhanced in developing cartilage and in chondrosarcoma (Bosserhoff et al. (1997) Dev. Dyn. 208:516). Based on this data, a biological role for MIA in embryonic cell growth and morphogenesis has been suggested, and a therapeutic application of MIA in the development of an antitumor therapeutic has been proposed. Additionally, MIA expression correlates with progressive malignancy of melanocytic lesions (Bosserhoff et al. (1997) J. Biol. Chem. 271:490), and MIA protein levels are enhanced in serum of patients with malignant melanoma (Bosserhoff et al. (1997) Cancer Res. 57:3149), supporting another proposed use of MIA as a marker of cancer progression.

The bovine orthologue of this molecule, named CD-RAP for cartilage derived RA-sensitive protein, was independently shown to be downregulated in retinoic acid (RA)-treated chondrocytes (Dietz & Sandell (1996) J. Biol. Chem. 271:3311). Retinoic acid is involved in the growth and differentiation of a variety of tissues, including the central nervous system, skin, and skeleton. RA increases the expression of the transcription factor AP-2, which has been shown to regulate CD-RAP transcription. The regulation of CD-RAP by AP-2 and the observed effects of RA on CD-RAP expression suggest that CD-RAP may participate in growth or developmental processes regulated by RA, or in other RA-regulated processes.

Homologues of MIA/CD-RAP appear to be present in a variety of tissues, including rat mammary carcinoma (GenBank™ Accession Number U67884), mouse mammary gland (GenBank™ Accession Numbers AA982842 and AA960553), mouse hypothalamus (GenBank™ Accession Number AA967578), human fetal heart (GenBank™ Accession Numbers AA062943, AA035545, W94322, W74647, W75984). The discovery of MIA/CD-RAP homologues in cancer cells and developing tissues suggests a common developmental role for the members of this family.

### SUMMARY OF THE INVENTION

The present invention is based, at least in part, on the discovery of a mouse and human genes encoding TANGO 130, a secreted protein which shares homology to MIA. These proteins, fragments thereof, derivatives thereof, and variants thereof are collectively referred to herein as the polypeptides of the invention or the proteins of the invention. Nucleic acid molecules encoding polypeptides of the invention are collectively referred to as nucleic acids of the invention.

The mouse TANGO 130 cDNA described below (SEQ ID NO:1) has a 2145 nucleotide open reading frame (nucleotides 24-2168 of SEQ ID NO:1; SEQ ID NO:2) which encodes a 714 amino acid protein (SEQ ID NO:3). This protein includes a predicted signal sequence of about 24 amino acids (from amino acid 1 to about amino acid 24 of SEQ ID NO:3; SEQ ID NO:5) and a predicted mature protein of about 690 amino acids (from about amino acid 25 to amino acid 714 of SEQ ID NO:3; SEQ ID NO:4).

The partial human TANGO 130 described below (SEQ ID NO:7) has a 1230 nucleotide open reading frame (nucleotides 34 to 1263 of SEQ ID NO:7; SEQ ID NO:8) which encodes a 410 amino acid protein (SEQ ID NO:9). The protein includes a predicted signal sequence of about amino acids (from amino acid 1 to about amino acid 23 of SEQ ID NO:10; SEQ ID NO:11) and a predicted mature protein of about 387 amino acids (from about amino acid 24 to amino acid 387 of SEQ ID NO:9; SEQ ID NO:10).

The full length human TANGO 130 described below (SEQ ID NO:14) has a 8121 nucleotide open reading frame (nucleotides 5 to 5725 of SEQ ID NO:14; SEQ ID NO:16) which encodes a 1907 amino acid protein (SEQ ID NO:15). The protein includes a predicted signal sequence of about amino acids (from amino acid 1 to about amino acid 23 of SEQ ID NO:16; SEQ ID NO:18) and a predicted mature protein of about 1884 amino acids (from about amino acid 24 to amino acid 1907 of SEQ ID NO:15; SEQ ID NO:17).

Mouse and human TANGO 130 proteins possess a MIA homology domain (described below) at their amino terminus (amino acids 1-125; SEQ ID NO:6 shows mouse, SEQ ID NO:12 shows human). The MIA homology domain contains four cysteines, conserved in both human and mouse TANGO 130 and is believed to be important to the molecules' structure and function. The amino acid sequence of the mouse TANGO 130 MIA homology domain is 36% identical to human MIA, while the human TANGO 130 MIA homology domains are 38% identical to human MIA (the MIA domains are the same in both the partial and full length versions of the human MIA protein sequence).

MIA and its bovine orthologue, CD-RAP, participate in activities involving cellular proliferation. These molecules inhibit the growth of melanocytes and chondrosarcoma tumor cells, and therefore have therapeutic utility in the treatment of malignant melanoma and chondrosarcoma. Additionally, they are abundantly expressed in malignant melanoma and are useful as serum markers of metastatic melanoma. Thus, molecules related to MIA and CD-RAP, e.g., TANGO 130 molecules, can be used to treat patients with such metastatic tumors. Also, TANGO 130 molecules of the invention which are overexpressed in abnormal cells can be used as serum markers in the diagnosis and monitoring of associated disease states.

In addition to cellular proliferation, MIA and CD-RAP are active in the process of cellular differentiation, being found in developing cartilaginous tissues from the onset of chondrogenesis and throughout development. Additionally, CD-RAP expression is sensitive to retinoic acid, which functions in the growth and differentiation of the central nervous system, skin and skeleton. The transcription factor AP-2, which increases in response to retinoic acid, binds to the CD-RAP promoter and inhibits CD-RAP transcription. Thus, modulation of CD-RAP can result in the modulation of retinoic acid or AP-2 function. Molecules related to CD-RAP, e.g., TANGO 130 molecules, also function in cell differentiation pathways, e.g., pathways involving retinoic acid and/or AP-2. The finding of TANGO 130 expression in mouse embryo throughout development supports a role for TANGO 130 in cellular differentiation. TANGO 130 molecules of the invention which modulate the function of factors involved in cellular differentiation are therefore useful in modulating responses involved in related disorders, e.g., developmental disorders.

MIA also has therapeutic utility as an immunosuppressive agent. Interleukin 2-dependent and phytohaemagglutinin-induced proliferation of peripheral blood lymphocytes are suppressed by MIA, as is the cytotoxicity of T lymphocytes (Canadian Patent Application Number 2,167,693). Thus, TANGO 130 molecules of the invention which modulate the proliferation of immune cells have utility as modulators of immune function.

Accordingly, in one aspect, this invention provides isolated nucleic acid molecules encoding TANGO 130 proteins or biologically active portions thereof, as well as nucleic acid fragments suitable as primers or hybridization probes for the detection of TANGO 130-encoding nucleic acids.

The invention features a nucleic acid molecule which is at least 45% (or 55%, 65%, 75%, 85%, 95%, or 98%) identical to the nucleotide sequence shown in SEQ ID NO:1, SEQ ID NO:2, SEQ ID NO:7, SEQ ID NO:8, the nucleotide sequence of the cDNA insert of the plasmid deposited with ATCC as Accession Number 98823 (the “cDNA of ATCC 98823”), the nucleotide sequence of the cDNA insert of the plasmid deposited with ATCC as Accession Number 98844 (the “cDNA of ATCC 98844”), the nucleotide sequence of the cDNA insert of the plasmid deposited with ATCC as Accession Number 98845 (the “cDNA of ATCC 98845”), or a complement thereof.

The invention features a nucleic acid molecule which is at least 75% (or 78%, 80%, 82%, 85%, 88%, 90%, 92%, 95%, 96%, 97%, 98%, or 99%) identical to the nucleotide sequence shown in SEQ ID NO:14, SEQ ID NO:15, or a complement thereof.

The invention features a nucleic acid molecule which includes a fragment of at least 500 (550, 600, 650, 700, 800, 900, 1000, or 1290) nucleotides of the nucleotide sequence shown in SEQ ID NO:7, the nucleotide sequence of the cDNA of ATCC 98823 or a complement thereof; or includes a fragment of at least 500 (550, 600, 650, 700, 800, 900, 1000, 1250, 1500, 1750, 2000, 2500, or 3000) nucleotides of the nucleotide sequence shown in SEQ ID NO:1, the nucleotide sequence of the cDNA of ATCC 98844, the nucleotide sequence of the cDNA of ATCC 98845, or a complement thereof.

The invention features a nucleic acid molecule which includes a fragment of at least 5820 (5840, 5860, 5880, 5900, 5920, 5940, 5960, 5980, 6000, 6020, 6040, 6060, 6080, 6100, 6120, 6140, 6160, 6180, 6200, 6220, 6240, 6260, 6280, 6300, 6320, 6340, 6360, 6380, 6400, 6420, 6440, 6460, 6480, 6500, 6520, 6540, 6560, 6580, 6600, 6620, 6640, 6660, 6680, 6700, 6720, 6740, 6760, 6780, 6800, 6820, 6840, 6860, 6880, 6900, 6920, 6940, 6960, 6980, 7000, 7020, 7040, 7060, 7080, 7100, 7120, 7140, 7160, 7180, 7200, 7220, 7240, 7260, 7280, 7300, 7320, 7340, 7360, 7380, 7400, 7420, 7440, 7460, 7480, 7500, 7520, 7540, 7560, 7580, 7600, 7620, 7640, 7660, 7680, 7700, 7720, 7740, 7760, 7780, 7800, 7820, 7840, 7860, 7880, 7900, 7920, 7940, 7960, 7980, 8000, 8020, 8040, 8060, 8080, 8100, or 8121) nucleotides of the nucleotide sequence shown in SEQ ID NO:14, or a complement thereof.

The invention features a nucleic acid molecule which includes a fragment of at least 3610 (3620, 3640, 3660, 3680, 3700, 3720, 3740, 3760, 3780, 3800, 3820, 3840, 3860, 3880, 3900, 4020, 4040, 4060, 4080, 4100, 4120, 4140, 4160, 4180, 4200, 4220, 4240, 4260, 4280, 4300, 4320, 4340, 4360, 4380, 4400, 4420, 4440, 4460, 4480, 4500, 4520, 4540, 4560, 4580, 4600, 4620, 4640, 4660, 4680, 4700, 4720, 4740, 4760, 4780, 4800, 4820, 4840, 4860, 4880, 4900, 4920, 4940, 4960, 4980, 5000, 5020, 5040, 5060, 5080, 5100, 5120, 5140, 5160, 5180, 5200, 5220, 5240, 5260, 5280, 5300, 5320, 5340, 5360, 5380, 5400, 5420, 5440, 5460, 5480, 5500, 5520, 5540, 5560, 5580, 5600, 5620, 5640, 5660, 5680, 5700, or 5720) nucleotides of the nucleotide sequence shown in SEQ ID NO:15, or a complement thereof.

The invention also features a nucleic acid molecule which includes a nucleotide sequence encoding a protein having an amino acid sequence that is at least 45% (or 55%, 65%, 75%, 85%, 95%, or 98%) identical to the amino acid sequence of SEQ ID NO:3, SEQ ID NO:4, SEQ ID NO:5, SEQ ID NO:6, SEQ ID NO:9, SEQ ID NO:10, SEQ ID NO:11, SEQ ID NO:12, the amino acid sequence encoded by the cDNA of ATCC 98823, the amino acid sequence encoded by the cDNA of ATCC 98844, or the amino acid sequence encoded by the cDNA of ATCC 98845.

The invention also features a nucleic acid molecule which includes a nucleotide sequence encoding a protein having an amino acid sequence that is at least 65% (or 68%, 72%, 75%, 78%, 82%, 85%, 92%, 95%, or 98%) identical to the amino acid sequence of SEQ ID NO:16, SEQ ID NO:17, SEQ ID NO:18, or SEQ ID NO:19.

In a preferred embodiment, a TANGO 130 nucleic acid molecule has the nucleotide sequence shown SEQ ID NO:1, SEQ ID NO:2, SEQ ID NO:7, SEQ ID NO:8, SEQ ID NO:14, SEQ ID NO:15, the nucleotide sequence of the cDNA of ATCC 98823, the nucleotide sequence of the cDNA of ATCC 98844, or the nucleotide sequence of the cDNA of ATCC 98845.

Also within the invention is a nucleic acid molecule which encodes a fragment of a polypeptide having the amino acid sequence of SEQ ID NO:3, SEQ ID NO:9, or SEQ ID NO:16. The fragment includes at least 135 (200, 300, 400, 500, 600, 714) contiguous amino acids of SEQ ID NO:3 or the polypeptide encoded by the cDNA of ATCC 98844 or 98845; includes at least 15 (25, 30, 50, 100, 150, 200, 300, 410) contiguous amino acids of SEQ ID NO:9 or the polypeptide encoded by the cDNA of ATCC 98823; or includes at least 1200 (1220, 1240, 1260, 1280, 1300, 1320, 1340, 1360, 1380, 1400, 1420, 1440, 1460, 1480, 1500, 1520, 1540, 1560, 1580, 1600, 1620, 1640, 1660, 1680, 1700, 1720, 1740, 1760, 1780, 1800, 1820, 1840, 1860, 1880, 1900, or 1907) contiguous amino acids of SEQ ID NO:16.

The invention includes a nucleic acid molecule which encodes a naturally occurring allelic variant of a polypeptide comprising the amino acid sequence of SEQ ID NO:3, SEQ ID NO:4, SEQ ID NO:6, SEQ ID NO:9, SEQ ID NO:10, SEQ ID NO:12, SEQ ID NO:16, SEQ ID NO:17, SEQ ID NO:19, an amino acid sequence encoded by the cDNA of ATCC 98823, an amino acid sequence encoded by the cDNA of ATCC 98844, or an amino acid sequence encoded by the cDNA of ATCC 98845 wherein the nucleic acid molecule hybridizes to a nucleic acid molecule comprising SEQ ID NO:1, SEQ ID NO:7, SEQ ID NO:14, the cDNA of ATCC 98823, the cDNA of ATCC 98844, or the cDNA of ATCC 98845, or a complement thereof under stringent conditions.

Also within the invention are: an isolated TANGO 130 protein having an amino acid sequence that is at least about 45%, preferably 50%, 60%, 75%, 85%, 95%, or 98% identical to the amino acid sequence of SEQ ID NO:4, the amino acid sequence of SEQ ID NO:3, the amino acid sequence of SEQ ID NO:10 or SEQ ID NO:9; an isolated TANGO 130 protein having an amino acid sequence that is at least about 65%, preferably 68%, 72%, 75%, 78%, 82%, 85%, 88%, 92%, 95%, 98%, or 99% identical to the amino acid sequence of SEQ ID NO:17, the amino acid sequence of SEQ ID NO:16; and an isolated TANGO 130 protein having an amino acid sequence that is at least about 45%, 50%, 60%, 70%, 85%, 95%, or 98% identical to the MIA homology domain of SEQ ID NO:6, SEQ ID NO:12, or SEQ ID NO:19 (e.g., about amino acid residues 1 to 125 of SEQ ID NO:3, SEQ ID NO:9, or SEQ ID NO:16).

Also within the invention are: an isolated TANGO 130 protein which is encoded by a nucleic acid molecule having a nucleotide sequence that is at least about 45%, preferably 55%, 65%, 75%, 85%, or 95% identical to SEQ ID NO:2, SEQ ID NO:8, the cDNA of ATCC 98823, the cDNA of ATCC 98844, or the cDNA of ATCC 98845; an isolated TANGO 130 protein which is encoded by a nucleic acid molecule having a nucleotide sequence that is at least about 65%, preferably 68%, 72%, 75%, 78%, 82%, 85%, 88%, 92%, 95%, 98%, or 99% identical to SEQ ID NO:15; an isolated TANGO 130 protein which is encoded by a nucleic acid molecule having a nucleotide sequence at least about 45%, preferably 55%, 65%, 75%, 85%, or 95% identical to the MIA homology domain encoding portion of SEQ ID NO:1, SEQ ID NO:7, or SEQ ID NO:14 (e.g., about nucleotides 24 to 398 of SEQ ID NO:1, nucleotides 34 to 408 of SEQ ID NO:7, or nucleotides 5 to 379 of SEQ ID NO:14); and an isolated TANGO 130 protein which is encoded by a nucleic acid molecule having a nucleotide sequence which hybridizes under stringent hybridization conditions to a nucleic acid molecule having the nucleotide sequence of SEQ ID NO:2, SEQ ID NO:8, the cDNA of ATCC 98823, the cDNA of ATCC 98844, the cDNA of ATCC 98845, or a complement thereof.

Also within the invention is a polypeptide which is a naturally occurring allelic variant of a polypeptide that includes the amino acid sequence of SEQ ID NO:3, SEQ ID NO:4, SEQ ID NO:6, SEQ ID NO:9, SEQ ID NO:10, SEQ ID NO:12, SEQ ID NO:16, SEQ ID NO:17, SEQ ID NO:19, an amino acid sequence encoded by the cDNA insert of the plasmid deposited with ATCC as Accession Number 98823, an amino acid sequence encoded by the cDNA insert of the plasmid deposited with ATCC as Accession Number 98844, or an amino acid sequence encoded by the cDNA insert of the plasmid deposited with ATCC as Accession Number 98845, wherein the polypeptide is encoded by a nucleic acid molecule which hybridizes to a nucleic acid molecule comprising SEQ ID NO:1, SEQ ID NO:2, SEQ ID NO:7, SEQ ID NO:8, SEQ ID NO:14, SEQ ID NO:15, the cDNA of ATCC 98823, the cDNA of ATCC 98844, or the cDNA of ATCC 98845, or a complement thereof under stringent conditions.

Another embodiment of the invention features TANGO 130 nucleic acid molecules which specifically detect TANGO 130 nucleic acid molecules relative to nucleic acid molecules encoding related molecules, e.g., MIA or CD-RAP. For example, in one embodiment, a TANGO 130 nucleic acid molecule hybridizes under stringent conditions to a nucleic acid molecule comprising the nucleotide sequence of SEQ ID NO:1, SEQ ID NO:2, SEQ ID NO:7, SEQ ID NO:8, SEQ ID NO:14, SEQ ID NO:15, the cDNA of ATCC 98823, the cDNA of ATCC 98844, the cDNA of ATCC 98845, or a complement thereof. In another embodiment, the TANGO 130 nucleic acid molecule is at least 500 (550, 600, 650, 700, 800, 900, 1000, 1250, 1500, 1750, 2000, 2250, 2500, 2750, 3000, 3250, 3750, 4000, 4250, 4500, 4750, 5000, 5250, 550.0, 5750, 6000, 6250, 6500, 6750, 7000, 7250, 7500, 7750, or 8000) nucleotides in length and hybridizes under stringent conditions to a nucleic acid molecule comprising the nucleotide sequence shown in SEQ ID NO:1, SEQ ID NO:7, the cDNA of ATCC 98823, the cDNA of ATCC 98844, the cDNA of ATCC 98845, or a complement thereof. In a preferred embodiment, an isolated TANGO 130 nucleic acid molecule comprises nucleotides 24 to 398 of SEQ ID NO:1 (SEQ ID NO:6), nucleotides 34 to 408 of SEQ ID NO:7 (SEQ ID NO:12), or nucleotides 5 to 379 of SEQ ID NO:14 (SEQ ID NO:19) encoding the MIA homology domain of TANGO 130, or a complement thereof. In another embodiment, the invention provides an isolated nucleic acid molecule which is antisense to the coding strand of a TANGO 130 nucleic acid.

Another aspect of the invention provides a vector, e.g., a recombinant expression vector, comprising a TANGO 130 nucleic acid molecule of the invention. In another embodiment, the invention provides a host cell containing such a vector. The invention also provides a method for producing TANGO 130 protein by culturing, in a suitable medium, a host cell of the invention containing a recombinant expression vector such that a TANGO 130 protein is produced.

Another aspect of this invention features isolated or recombinant TANGO 130 proteins and polypeptides. Preferred TANGO 130 proteins and polypeptides possess at least one biological activity possessed by naturally occurring human TANGO 130, e.g., (1) the ability to form protein:protein interactions with proteins in the TANGO 130 signaling pathway; (2) the ability to bind TANGO 130 receptor; or (3) the ability to bind to an intracellular target. Other activities include: (1) the ability to modulate, e.g., inhibit, cell proliferation (e.g., proliferation of cells of the kidney, liver, heart, testis, immune system, skin, cartilage, skeleton, skeletal muscle and central nervous system), e.g., abnormal cell proliferation, e.g., malignant cell proliferation, e.g., malignant proliferation of epithelial cells (e.g., carcinomas, e.g., melanomas), malignant proliferation of mesenchymal cells (e.g., sarcomas, e.g., chondrosarcomas, glioblastomas); (2) the ability to modulate cell-cell interactions, e.g., cell adhesion, or cell-substrate interactions; (3) the ability to modulate cell migration, e.g., abnormal migration, e.g., metastasis of tumor cells; (4) the ability to modulate cell differentiation (e.g., differentiation of cells of the kidney, liver, heart, testis, immune system, skin, cartilage, skeleton, skeletal muscle and central nervous system); (5) the ability to modulate retinoic acid-mediated functions or activities, e.g., differentiation, e.g., differentiation of cells of the kidney, liver, heart, testis, immune system, skin, cartilage, skeleton, skeletal muscle and central nervous system, cell proliferation, e.g., proliferation of cells of the kidney, liver, heart, testis, immune system, skin, cartilage, skeleton, skeletal muscle and central nervous system or modulation of transcription factor function, e.g., modulation of AP-2 function; and (6) the ability to modulate embryonic cell growth and/or morphogenesis (e.g., of embryonic cells of the kidney, liver, heart, testis, immune system, skin, cartilage, skeleton, skeletal muscle and central nervous system).

The TANGO 130 proteins of the present invention, or biologically active portions thereof, can be operably linked to a non-TANGO 130 polypeptide (e.g., heterologous amino acid sequences) to form TANGO 130 fusion proteins. The invention further features antibodies that specifically bind TANGO 130 proteins, such as monoclonal or polyclonal antibodies. In addition, the TANGO 130 proteins or biologically active portions thereof can be incorporated into pharmaceutical compositions, which optionally include pharmaceutically acceptable carriers.

In another aspect, the present invention provides a method for detecting the presence of TANGO 130 activity or expression in a biological sample by contacting the biological sample with an agent capable of detecting an indicator of TANGO 130 activity such that the presence of TANGO 130 activity is detected in the biological sample.

In another aspect, the invention provides a method for modulating TANGO 130 activity comprising contacting a cell with an agent that modulates (inhibits or stimulates) TANGO 130 activity or expression such that TANGO 130 activity or expression in the cell is modulated. In one embodiment, the agent is an antibody that specifically binds to TANGO 130 protein. In another embodiment, the agent modulates expression of TANGO 130 by modulating transcription of a TANGO 130 gene, splicing of a TANGO 130 mRNA, or translation of a TANGO 130 mRNA. In yet another embodiment, the agent is a nucleic acid molecule having a nucleotide sequence that is antisense to the coding strand of the TANGO 130 mRNA or the TANGO 130 gene.

In one embodiment, the methods of the present invention are used to treat a subject having a disorder characterized by aberrant TANGO 130 protein activity or nucleic acid expression by administering an agent which is a TANGO 130 modulator to the subject. In one embodiment, the TANGO 130 modulator is a TANGO 130 protein. In another embodiment, the TANGO 130 modulator is a TANGO 130 nucleic acid molecule. In other embodiments, the TANGO 130 modulator is a peptide, peptidomimetic, or other small molecule. In a preferred embodiment, the disorder characterized by aberrant TANGO 130 protein or nucleic acid expression is marked by abnormal cellular growth, e.g., abnormal growth of the kidney, liver (e.g., regenerative disorders, liver atrophy, necrosis, cirrhosis or fibrosis), heart (e.g., cardiac damage following radiation therapy, hypertension, atherosclerosis or ischemic heart disease), central nervous system (e.g., motor neuron disease, multiple sclerosis, or degenerative disorders of the brain, e.g., Parkinson's or Alzheimer's diseases), cartilage (e.g., achondroplasia or osteoarthritis), skeleton (e.g., osteoporosis or fibrous dysplasia), skeletal muscle (e.g., fibromatoses), skin (e.g., psoriasis), immune system (e.g., inflammation or autoimmune disease) or testis (testicular atrophy).

Disorders characterized by abnormal cell growth also include cancer, e.g., primary or metastatic cancer of, e.g., the kidney, liver (e.g., hepatocellular carcinoma, cholangiocarcinoma, hepatoblastoma, angiosarcoma, or metastatic tumors of the liver), heart (e.g., primary sarcomas, e.g., lymphomas, or metastasis from cervical carcinoma), central nervous system (e.g., metastatic tumors (e.g., from lung, breast, kidney or the gastrointestinal tract, or metastatic tumors from melanoma), primary lymphomas, gliomas (e.g., astrocytomas or glioblastoma multiforme), or neuroblastomas), cartilage, skeleton (e.g., osteosarcoma, chondrosarcoma or Ewing's sarcoma), skeletal muscle (e.g., primary skeletal muscle lymphoma or rhabdomyosarcomas), skin (e.g., malignant melanoma), immune system or testis (e.g., seminomas or embryonal carcinomas).

Additionally, disorders characterized by aberrant TANGO 130 protein activity or nucleic acid expression may be marked by abnormal development, e.g., developmental disorders or abnormalities of the kidney, liver, heart, central nervous system, cartilage, skeleton, skeletal muscle, skin, immune system or testis, e.g., metabolic disorders (e.g., obesity, Gaucher's disease, or hemochromatosis), cardiac abnormalities (e.g., cardiac hypertrophy, cardiomyopathies, restenosis or congenital heart disease), neurodevelopmental disorders (e.g., developmental malformations of the central nervous system), chondrogenic disorders, skeletal abnormalities (e.g., Paget's disease), muscular atrophic or dystrophic disorders (e.g., congenital myopathy or muscular dystrophy), skin disorders, or sexual differentiation or dysfunction disorders (e.g., spermatogenesis, male infertility, undescended testis, gonadal dysgenesis, or androgen insensitivity syndrome).

The present invention also provides a diagnostic assay for identifying the presence or absence of a genetic lesion or mutation characterized by at least one of: (i) aberrant modification or mutation of a gene encoding a TANGO 130 protein; (ii) mis-regulation of a gene encoding a TANGO 130 protein; and (iii) aberrant post-translational modification of a TANGO 130 protein, wherein a wild-type form of the gene encodes a protein with a TANGO 130 activity.

In another aspect, the invention provides a method for identifying a compound that binds to or modulates the activity of a TANGO 130 protein. In general, such methods entail measuring a biological activity of a TANGO 130 protein in the presence and absence of a test compound and identifying those compounds which alter the activity of the TANGO 130 protein.

The invention also features methods for identifying a compound which modulates the expression of TANGO 130 by measuring the expression of TANGO 130 in the presence and absence of a compound.

Other features and advantages of the invention will be apparent from the following detailed description and claims.

### BRIEF DESCRIPTION OF THE DRAWINGS

FIGS. 1A-1D depict the cDNA sequence (SEQ ID NO:1) and predicted amino acid sequence (SEQ ID NO:3) of mouse TANGO 130. The open reading frame of SEQ ID NO:1 extends from nucleotide 24 to nucleotide 2165 (SEQ ID NO:2).

FIG. 2 is a hydropathy plot of mouse TANGO 130. The region encoding the signal sequence is indicated (“sp”), as are the position of cysteines (“cys”; small triangles immediately below the plot). Also shown is the region of homology to MIA and CD-RAP (“MIA”), hereinafter the “MIA homology domain”. Relative hydrophobicity is shown below the line marked “0”, and relative hydrophilicity is shown above the line marked “0”.

FIGS. 3A-3B depict the cDNA sequence (SEQ ID NO:7) and predicted amino acid sequence (SEQ ID NO:9) of partial human TANGO 130. The open reading frame of SEQ ID NO:7 extends from nucleotide 34 to nucleotide 1263 (SEQ ID NO:8).

FIG. 4 is a hydropathy plot of partial human TANGO 130. The region encoding the signal sequence is indicated (“sp”), as are the position of cysteines (“cys”; small triangles immediately below the plot). Also shown is the MIA homology domain (“MIA”). Relative hydrophobicity is shown below the line marked “0”, and relative hydrophilicity is shown above the line marked “0”.

FIG. 5 depicts an amino acid alignment of the MIA homology domain of human and mouse TANGO 130 (“Human T130 MIA”, SEQ ID NO:12 and “Mouse T130 MIA”, SEQ ID NO:6; corresponding to amino acids 1 to 125 of SEQ ID NO:9 and 3, respectively) with human MIA (GenBank™ Accession Number Q16674), mouse MIA (GenBank™ Accession Number Q61865), rat MIA (GenBank™ Accession Number U678.84) and bovine CD-RAP (GenBank™ Accession Number Q28038).

FIGS. 6A-6J depict the cDNA sequence (SEQ ID NO:14) and predicted amino acid sequence (SEQ ID NO:16) of full length human TANGO 130. The open reading frame of SEQ ID NO:14 extends from nucleotide 5 to nucleotide 5725 (SEQ ID NO:15).

FIG. 7 is a hydropathy plot of full length human TANGO 130. In the hydrophobicity plots disclosed herein, the locations of cysteine residues (“Cys”) and potential N-glycosylation sites (“Ngly”) are indicated by vertical bars and the predicted extracellular (“out”), intracellular (“ins”), or transmembrane (“TM”) portions of the protein backbone are indicated by a horizontal bar. Relatively hydrophobic regions of the protein are above the dashed horizontal line, and relatively hydrophilic regions of the protein are below the dashed horizontal line.

### DETAILED DESCRIPTION OF THE INVENTION

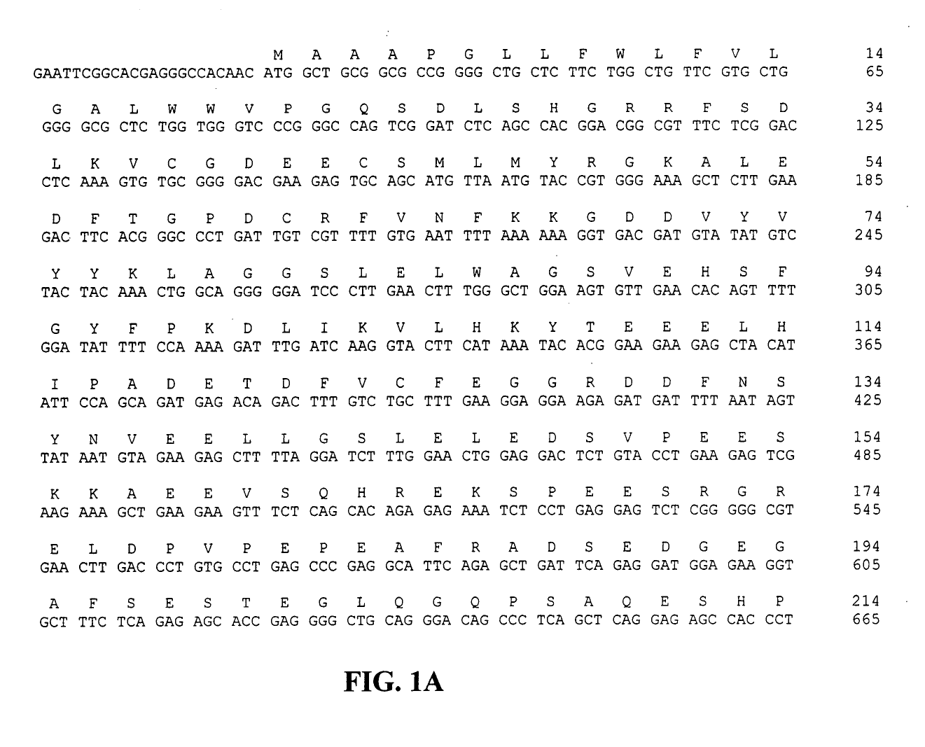

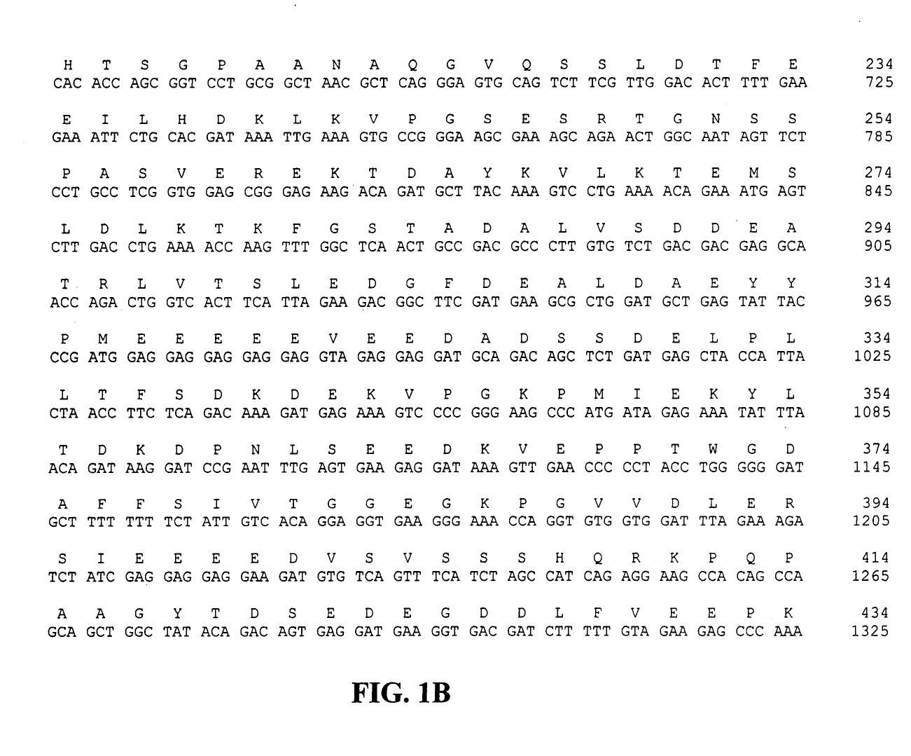

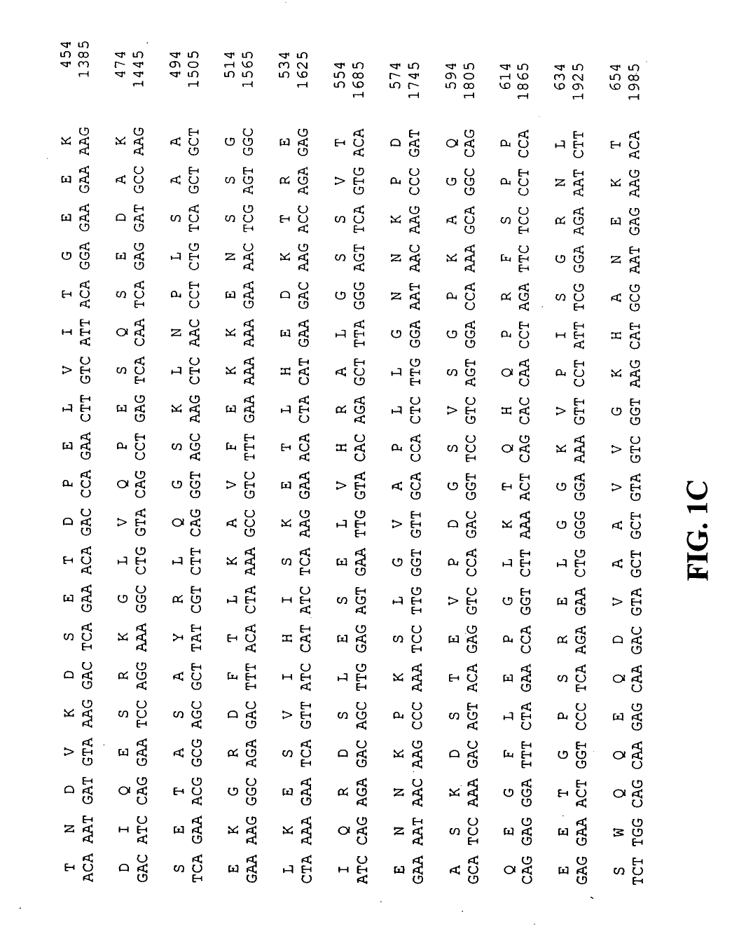

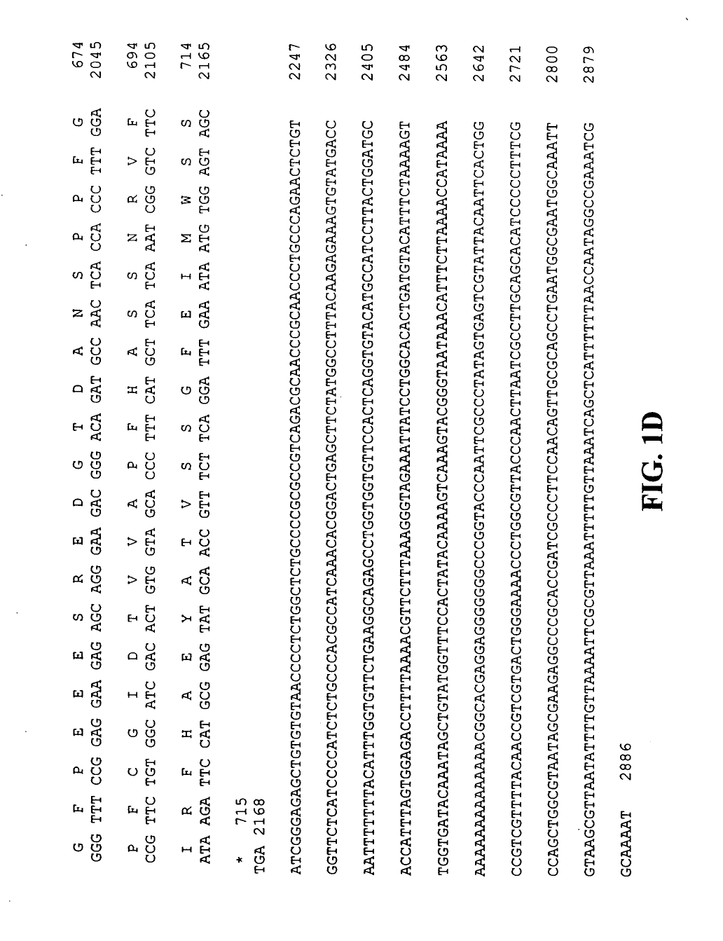

The present invention is based on the discovery of cDNA molecules encoding mouse and human TANGO 130 which share homology to MIA. A nucleotide sequence encoding a mouse TANGO 130 protein is shown in FIGS. 1A-1D (SEQ ID NO:1; SEQ ID NO:2 includes the open reading frame only). A predicted amino acid sequence of mouse TANGO 130 protein is also shown in FIGS. 1A-1D (SEQ ID NO:3). The mouse TANGO 130 cDNA is approximately 2886 nucleotides long including untranslated regions and encodes a 714 amino acid protein having a molecular weight of approximately 78.5 kDa (excluding post-translational modifications). The cDNA encodes a predicted amino terminal signal peptide sequence of approximately 24 amino acids (SEQ ID NO:5), the cleavage of which leaves an approximately 690 amino acid mature polypeptide (SEQ ID NO:4) with a molecular weight of approximately 75.9 kDa (excluding post-translational modifications). A plasmid containing nucleotides 1 to 1555 of murine TANGO 130 cDNA (with the cDNA insert name of “mamaMIA.mouse.5′”) was deposited with American Type Culture Collection (ATCC), Manassas, Va. on Aug. 21, 1998 and assigned Accession Number 98844. A plasmid containing nucleotides 1264-2886 of murine TANGO 130 cDNA (with the cDNA insert name of “mamaMIA.mouse.3′”) was deposited with American Type Culture Collection (ATCC), Manassas, Va. on Aug. 21, 1998 and assigned Accession Number 98845.

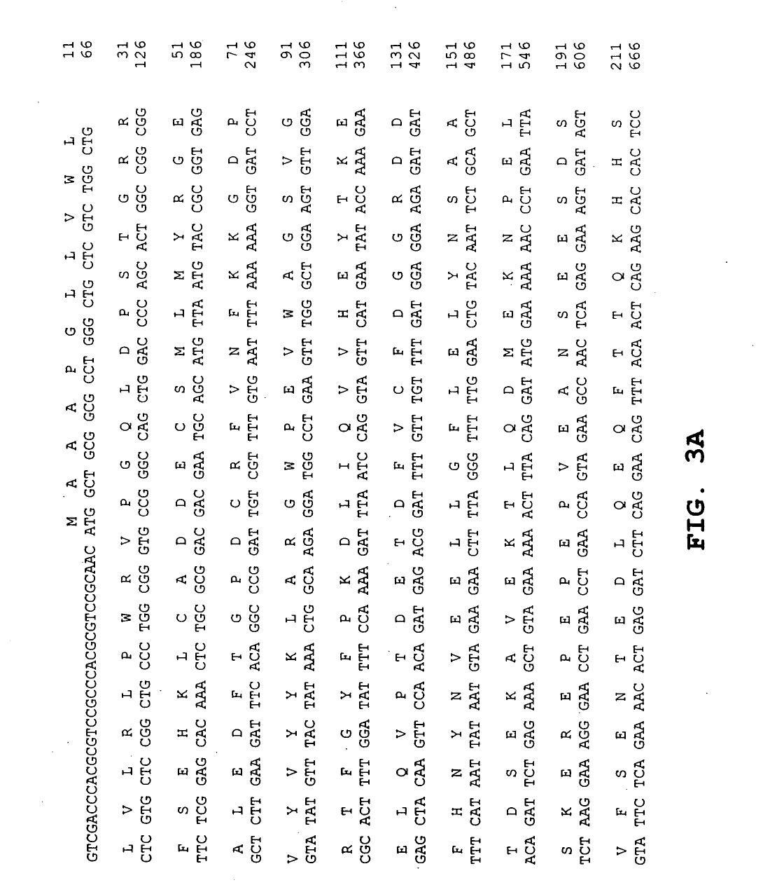

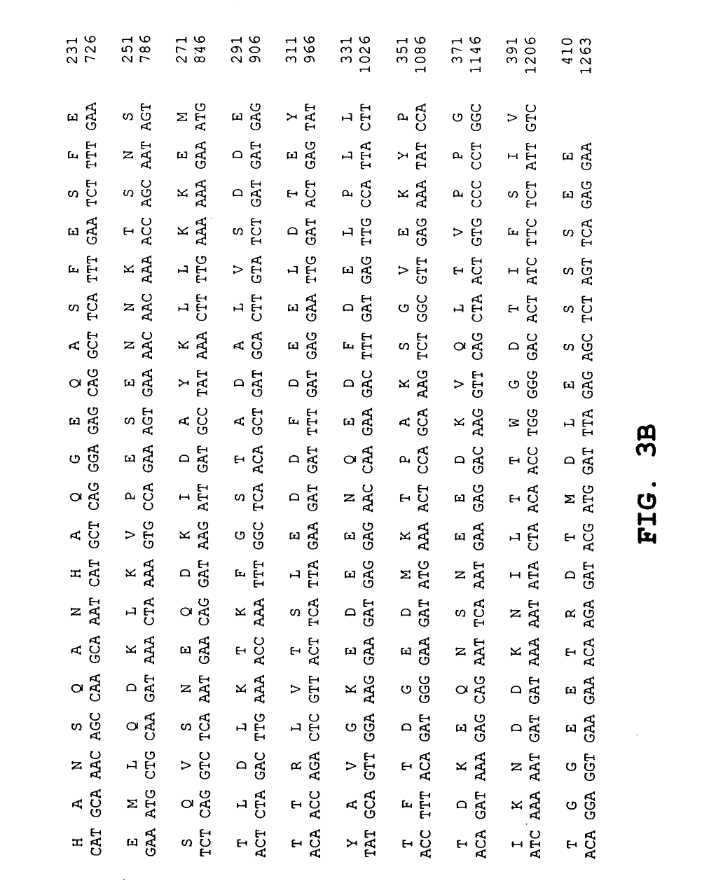

A nucleotide sequence encoding partial human TANGO 130 protein is shown in FIGS. 3A-3B (SEQ ID NO:7; SEQ ID NO:8 includes the open reading frame only). A predicted amino acid sequence of partial human TANGO 130 protein is also shown in FIGS. 3A-3B (SEQ ID NO:9). The human partial TANGO 130 cDNA is approximately 1263 nucleotides long including untranslated regions and encodes a partial protein having a molecular weight of approximately 46.5 kDa (excluding post-translational modifications). The sequence encodes a predicted amino terminal signal peptide sequence of approximately 23 amino acids (SEQ ID NO:11), the cleavage of which leaves an approximately 387 amino acid mature polypeptide (SEQ ID NO:10) with a molecular weight of approximately 44.0 kDa (excluding post-translational modifications). A plasmid containing a cDNA encoding partial human TANGO 130 (with the cDNA insert name of “mamaMIA”) was deposited with American Type Culture Collection (ATCC), Manassas, Va. on Jul. 21, 1998 and assigned Accession Number 98823. These deposits will be maintained under the terms of the Budapest Treaty on the International Recognition of the Deposit of Microorganisms for the Purposes of Patent Procedure. These deposits were made merely as a convenience for those of skill in the art and are not an admission that a deposit is required under 35 U.S.C. §112.

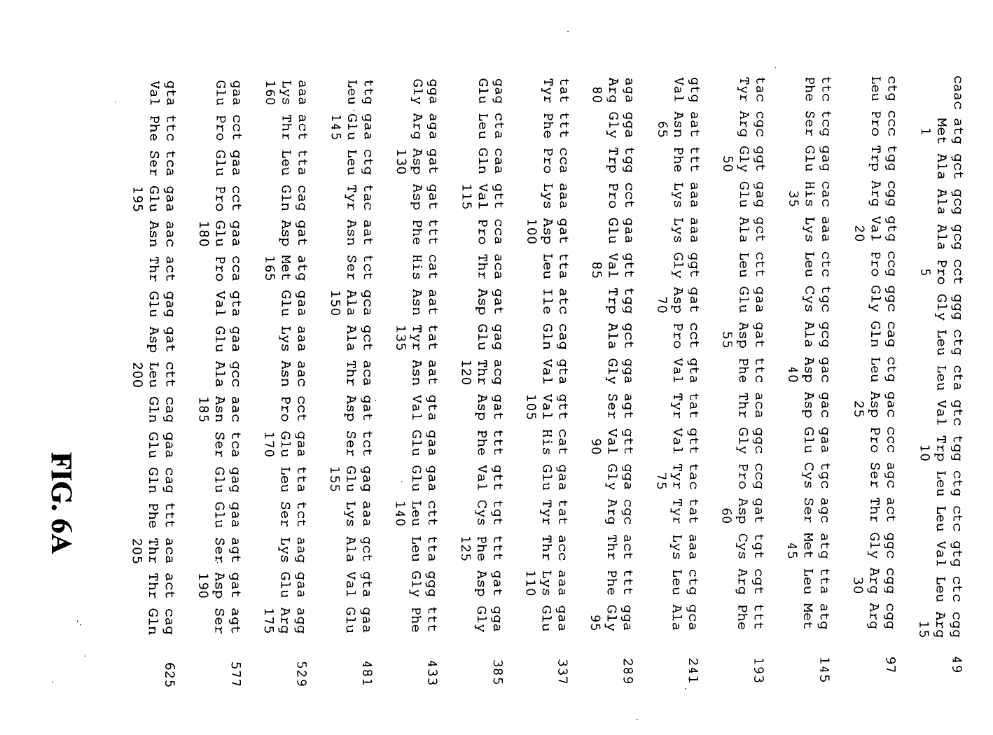

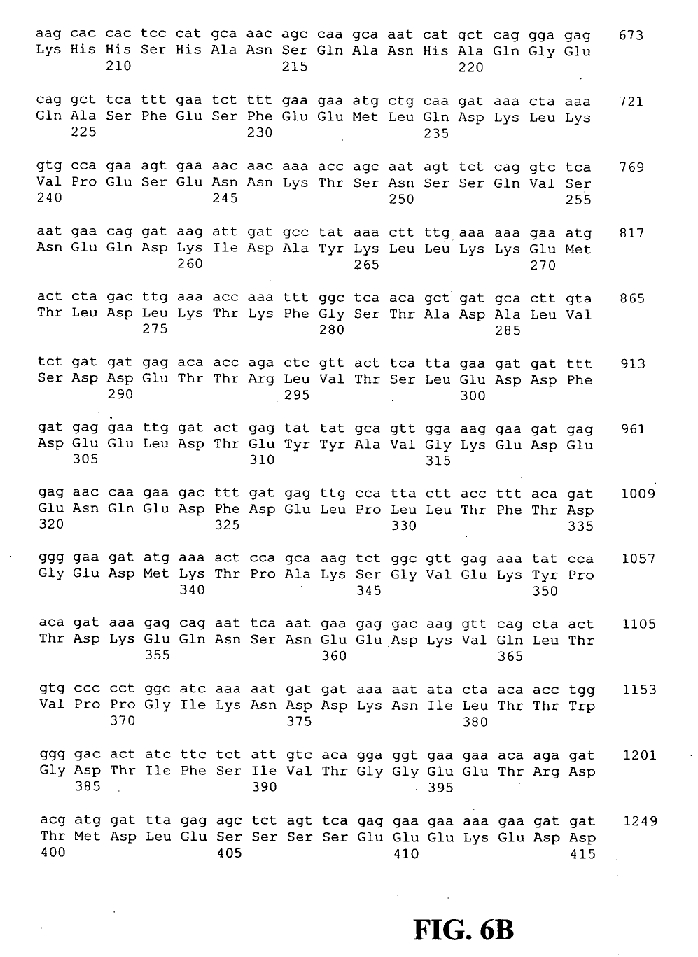

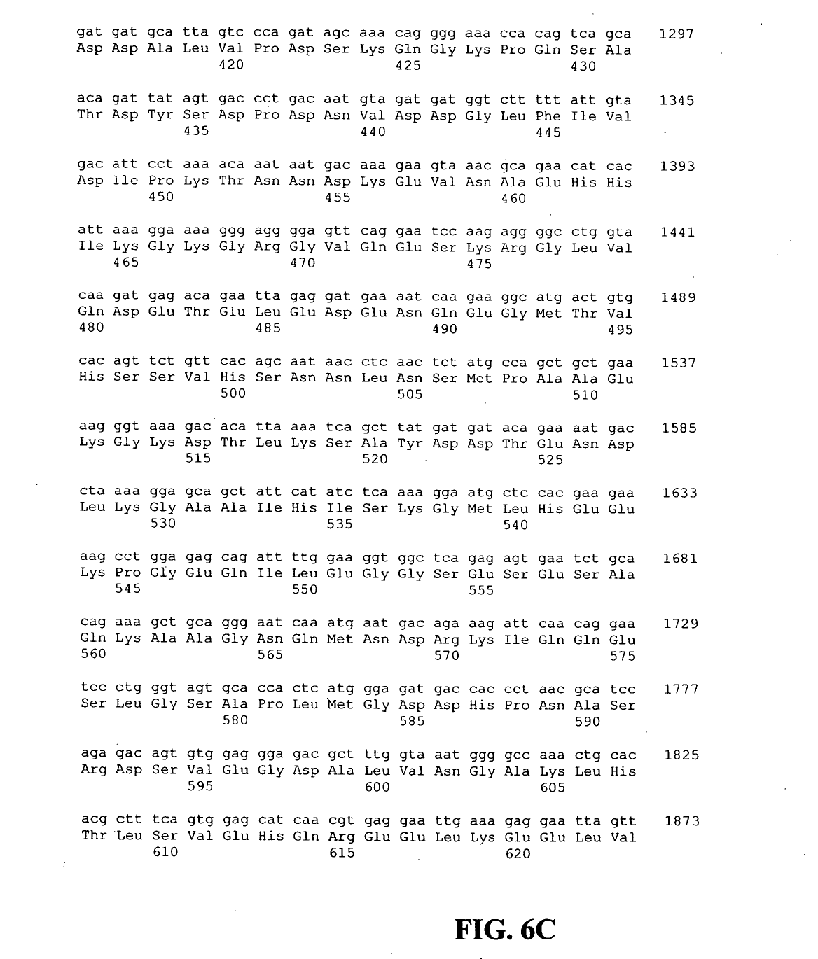

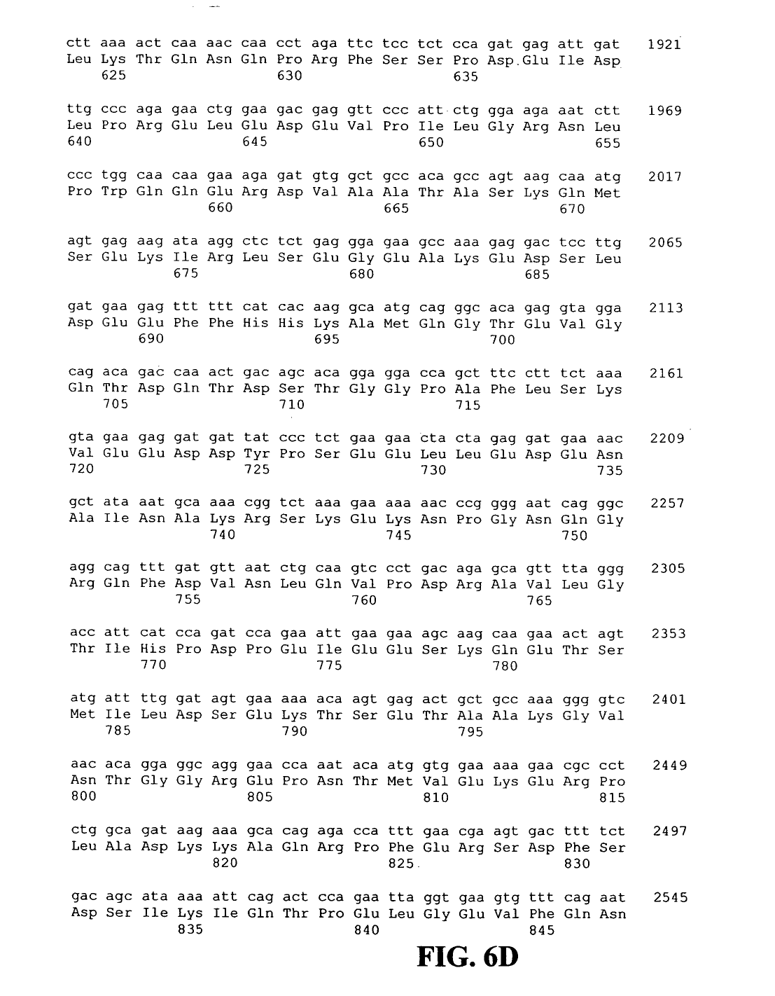

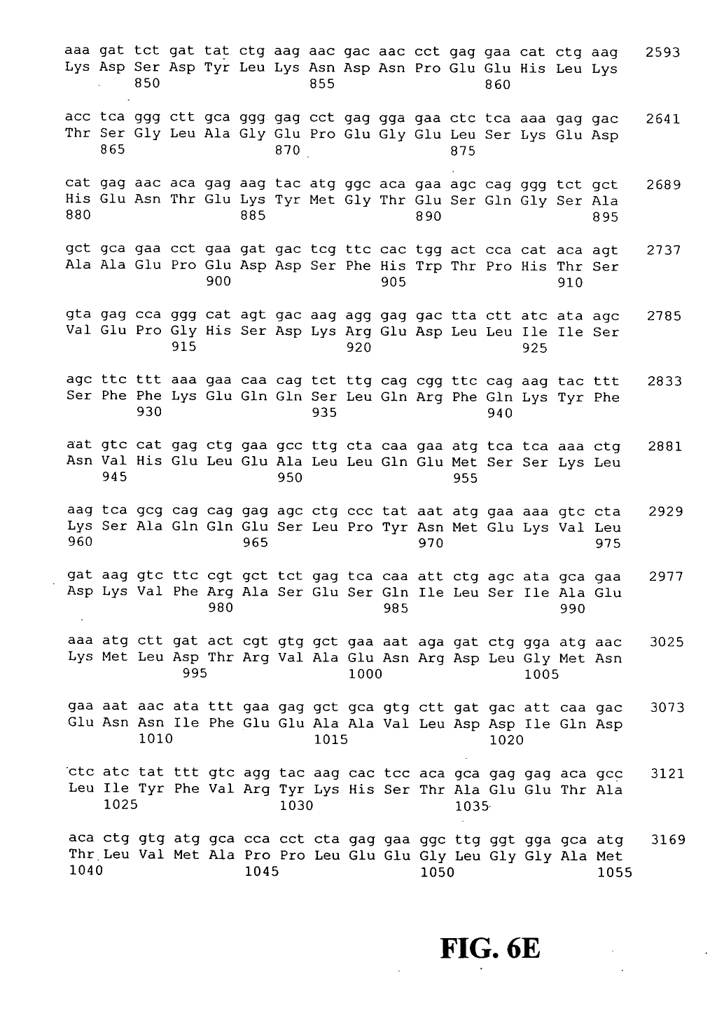

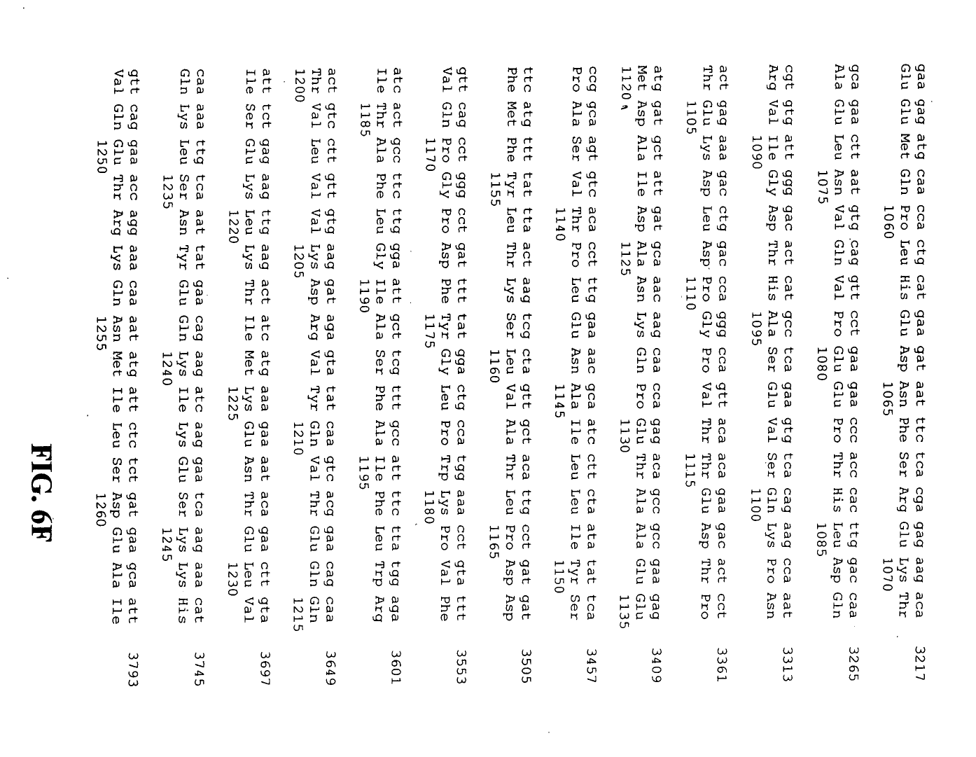

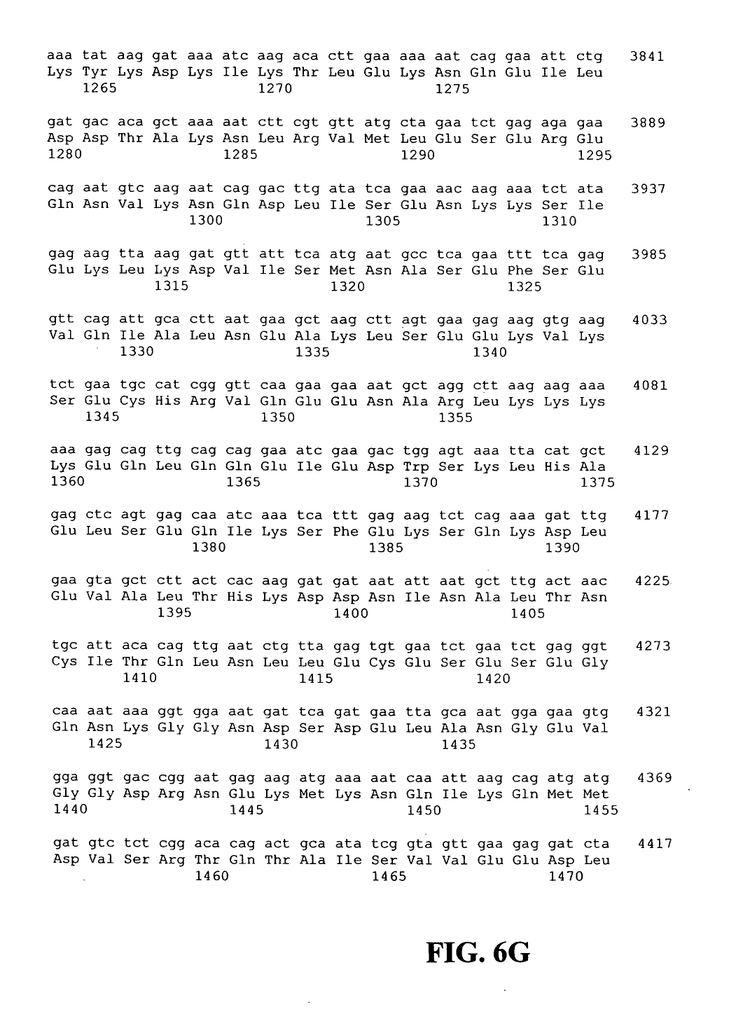

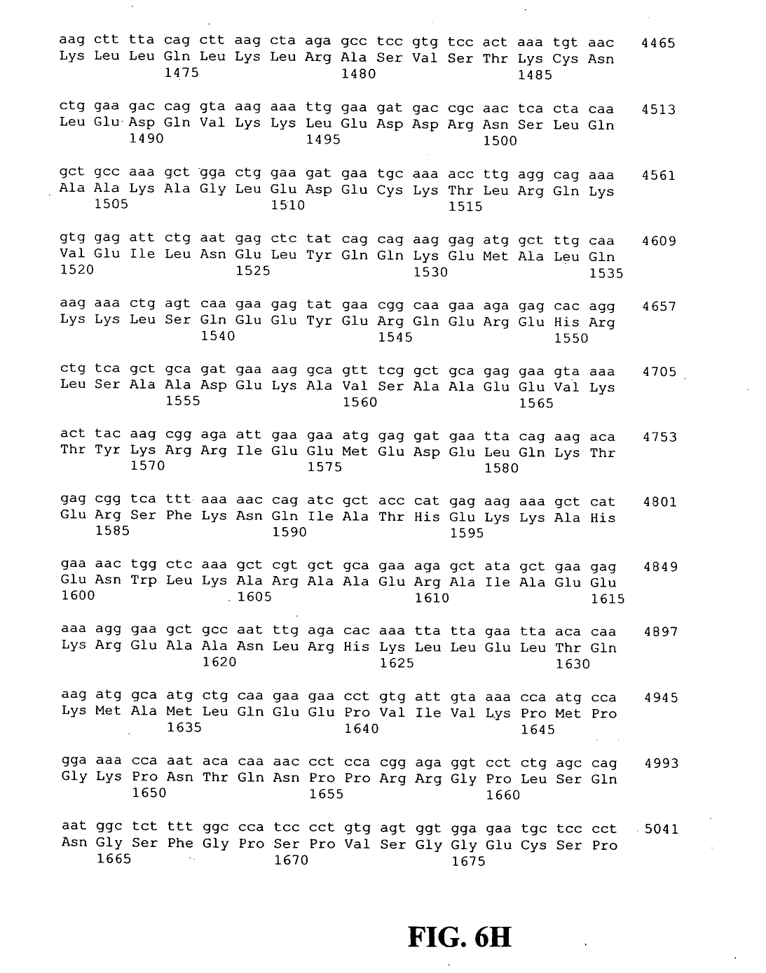

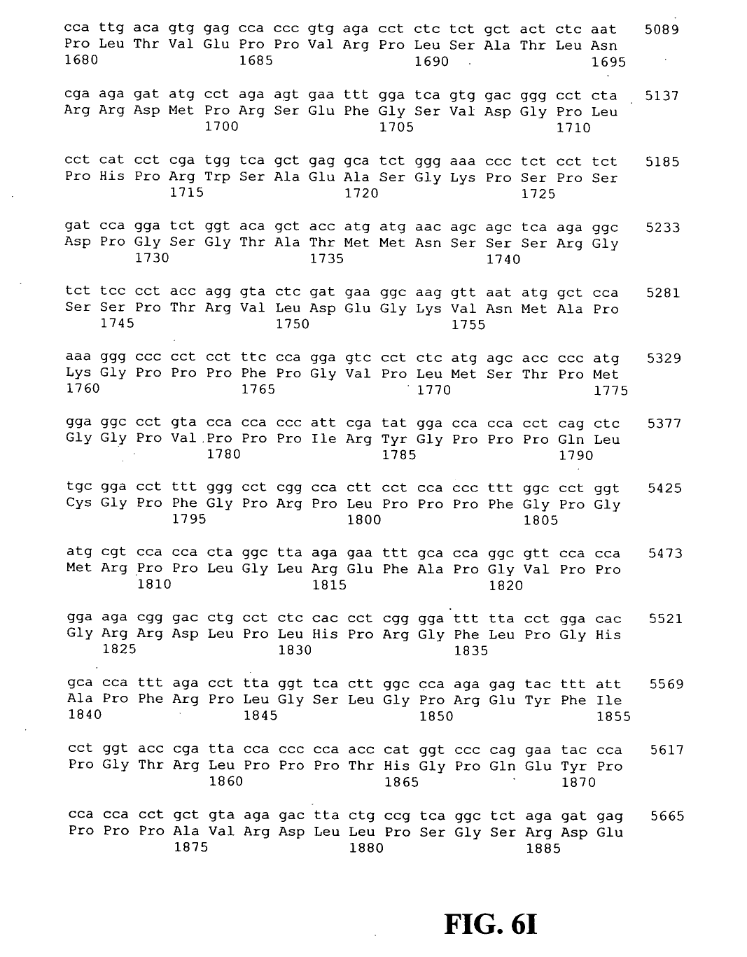

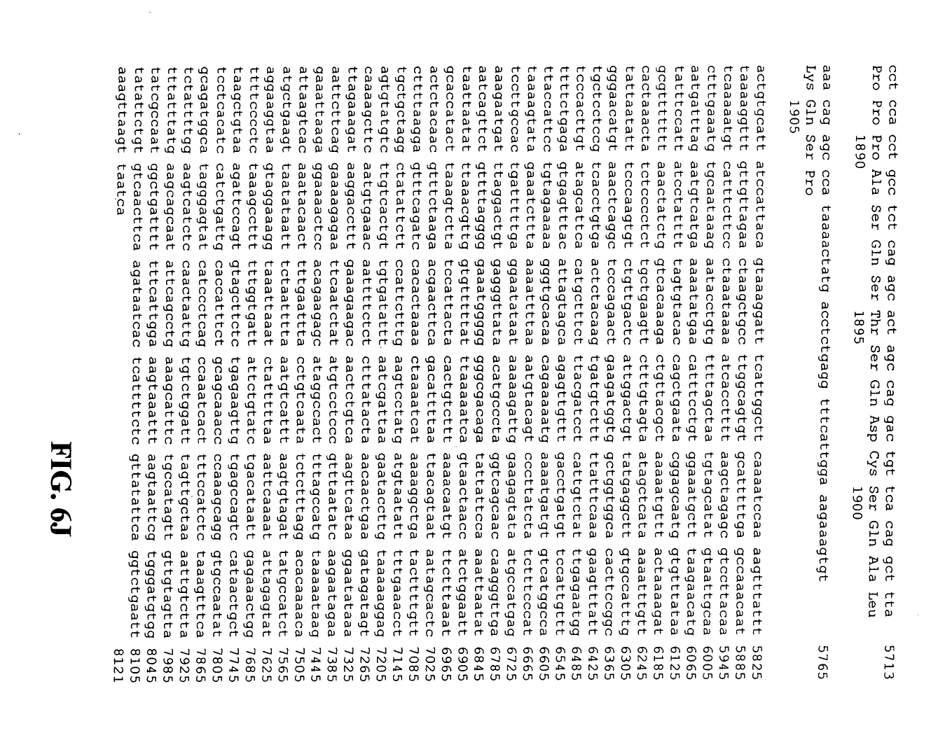

A nucleotide sequence encoding full length human TANGO 130 protein is shown in FIGS. 6A-6J (SEQ ID NO:14; SEQ ID NO:15 includes the open reading frame only). A predicted amino acid sequence of full length human TANGO 130 protein is also shown in FIGS. 6A-6J (SEQ ID NO:16). The full length human TANGO 130 cDNA is approximately 8121 nucleotides long including untranslated regions and encodes a partial protein having a molecular weight of approximately 213.7 kDa (excluding post-translational modifications). The sequence encodes a predicted amino terminal signal peptide sequence of approximately 23 amino acids (SEQ ID NO:18), the cleavage of which leaves an approximately 1884 amino acid mature polypeptide (SEQ ID NO:17) with a molecular weight of approximately 211.2 kDa (excluding post-translational modifications).

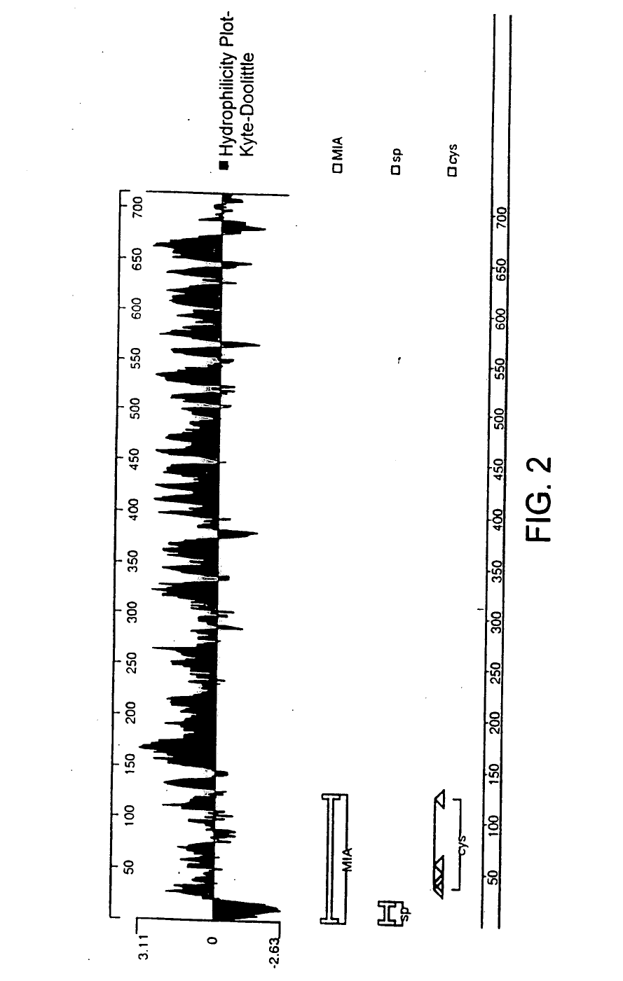

One embodiment of the invention features TANGO 130 molecules which contain a signal sequence. As used herein, a signal sequence (or signal peptide) includes a peptide of at least about 10 amino acid residues in length which occurs at the amino terminus of membrane-bound and secreted proteins and which contains at least about 45% hydrophobic amino acid residues such as alanine, leucine, isoleucine, phenylalanine, proline, tyrosine, tryptophan, or valine. In one embodiment, a signal sequence contains at least about 10 to 35 amino acid residues, and has at least about 35-60%, more preferably 40-50%, and more preferably at least about 45% hydrophobic residues. A signal sequence serves to direct a protein containing such a sequence to a lipid bi-layer. Thus, in one embodiment, a TANGO 130 protein contains a signal sequence corresponding to about amino acid residues 1 to 23 of SEQ ID NOs:9 and 16, and about amino acid residues 1 to 24 of SEQ ID NO:3 (i.e., SEQ ID NOs:11 and 18, and SEQ ID NO:5, respectively). It is recognized that the carboxyl terminal boundary of the signal sequence can be located one or two residues from the residue identified above (i.e., following residues 24, 25, 26, 27, or 28 of SEQ ID NOs:11 and 18, and following residues 25, 26, 27, 28, or 29 of SEQ ID NO:3). The signal sequence is cleaved during processing of the mature protein. The predicted signal sequences of human and mouse TANGO 130, for example, are strongly hydrophobic sequences of about 23 and 24 amino acids, respectively, as shown in the TANGO 130 hydropathy plots (FIGS. 2, 4, and 7).

TANGO 130 proteins typically comprise a variety of potential post-translational modification sites (often within an extracellular domain), such as those described herein, as predicted by computerized sequence analysis of TANGO 130 proteins using amino acid sequence comparison software (comparing the amino acid sequence of TANGO 130 with the information in the PROSITE database {rel. 12.2; Februrary, 1995} and the Hidden Markov Models database {Rel. PFAM 3.3}). The predicted post translational modification sites for murine TANGO 130 protein include: predicted N-glycosylation sites (Pfam accession number PS00001) at about amino acid residues 360-363 and 631-634 of SEQ ID NO:3; a predicted cAMP- and cGMP-dependent protein kinase phosphorylation sites (Pfam accession number PS00004) at about amino acid residues 30-33 of SEQ ID NO:3; predicted protein kinase C phosphorylation sites (Pfam accession number PS00005) at about amino acid residues 154-156, 338-340, 355-357, 459-461, 501-503, 628-630, and 688-690 of SEQ ID NO:3; predicted casein kinase II phosphorylation sites (Pfam accession number PS00006) located at about amino acid residues 57-60, 109-112, 149-152, 167-170, 208-211, 228-231, 232-235, 283-286, 290-293, 299-302, 328-331, 336-339, 355-358, 362-365, 371-374, 381-384, 395-398, 419-422, 441-444, 450-453, 468-471, 492-495, 524-527, 550-553, 610-613, 659-662, and 704-707 of SEQ ID NO:3; a predicted tyrosine kinase phosphorylation site (Pfam accession number PS00008) at about amino acid residues 67-75 of SEQ ID NO:3; predicted N-myristoylation sites (Pfam accession number PS00008) at about amino acid residues 205-210, 225-230, 512-517, 548-553, 591-596, and 663-668 of SEQ ID NO:3; and a predicted amidation site (Pfam accession number PS00009) at about amino acid residues 28-31 of SEQ ID NO:3.

The predicted post translational modification sites for partial human TANGO 130 protein include: predicted N-glycosylation sites (Pfam accession number PS00001) at about amino acid residues 246-249 and 250-253 of SEQ ID NO:1; a predicted cAMP- and cGMP-dependent protein kinase phosphorylation sites (Pfam accession number PS00004) at about amino acid residues 30-33 of SEQ ID NO:1; predicted protein kinase C phosphorylation sites (Pfam accession number PS00005) at about amino acid residues 28-30, 154-156, 206-208, 292-294, and 352-354 of SEQ ID NO:11; predicted casein kinase II phosphorylation sites (Pfam accession number PS00006) located at about amino acid residues 57-60, 109-112, 152-155, 161-164, 229-232, 281-284, 297-300, 332-335, 345-348, 352-355, 358-361, 382-385, 392-395, and 406-409 of SEQ ID NO:11; a predicted tyrosine kinase phosphorylation site (Pfam accession number PS00008) at about amino acid residues 67-73 of SEQ ID NO:11; a predicted N-myristoylation site (Pfam accession number PS00008) at about amino acid residues 393-398 of SEQ ID NO:11; a predicted amidation site (Pfam accession number PS00009) at about amino acid residues 28-31 of SEQ ID NO:11; and a predicted aminoacyl-transfer RNA synthetase class II signature site (Pfam accession number PS00179) at about amino acid residues 48-70 of SEQ ID NO:11.

The predicted post translational modification sites for full length human TANGO 130 protein include: predicted N-glycosylation sites (Pfam accession number PS00001) at about amino acid residues 246-249, 250-253, 589-592, 1065-1068, 1321-1324, 1429-1432, 1664-1667, and 1738-1741 of SEQ ID NO:16; predicted cAMP- and cGMP-dependent protein kinase phosphorylation sites (Pfam accession number PS00004) at about amino acid residues 30-33 and 1536-1539 of SEQ ID NO:16; predicted protein kinase C phosphorylation sites (Pfam accession number PS00005) at about amino acid residues 28-30, 154-156, 206-208, 292-294, 352-354, 474-476, 516-518, 672-674, 788-790, 833-835, 883-885, 917-919, 956-958, 1099-1101, 1104-1106, 1217-1219, 1244-1246, 1251-1253, 1282-1284, 1292-1294, 1387-1389, 1396-1398, 1483-1485, 1515-1517, 1568-1570, 1583-1585, 1586-1588, 1630-1632, 1721-1723, and 1740-1742 of SEQ ID NO:16; predicted casein kinase II phosphorylation sites (Pfam accession number PS00006) located at about amino acid residues 57-60, 109-112, 152-155, 161-164, 229-232, 281-284, 297-300, 332-335, 345-348, 352-355, 358-361, 382-385, 392-395, 406-409, 430-433, 435-438, 452-455, 483-486, 519-522, 524-527, 554-557, 633-636, 678-681, 686-689, 718-721, 778-781, 808-811, 876-879, 910-913, 988-991, 1033-1036, 1083-1086, 1104-1107, 1114-1117, 1118-1121, 1131-1134, 1140-1143, 1163-1166, 1235-1238, 1292-1295, 1387-1390, 1396-1399, 1419-1422, 1465-1468, 1539-1542, 1553-1556, 1561-1564, 1673-1676, 1725-1728, 1884-1887, and 1895-1898 of SEQ ID NO:16; predicted tyrosine kinase phosphorylation sites (Pfam accession number PS00008) at about amino acid residues 67-73 and 719-725 of SEQ ID NO:16; predicted N-myristoylation sites (Pfam accession number PS00008) at about amino acid residues 393-398, 470-475, 552-557, 564-569, 699-704, 798-803, 893-898, 1005-1010, 1050-1055, 1423-1428, 1440-1445, 1730-1735, 1743-1748, and 1820-1825 of SEQ ID NO:16; predicted amidation sites (Pfam accession number PS00009) at about amino acid residues 28-31 and 1823-1826 of SEQ ID NO:16; a predicted leucine zipper pattern (Pfam accession number PS00029) at about amino acid residues 1488-1509 of SEQ ID NO:16; and a predicted aminoacyl-transfer RNA synthetase class II signature site (Pfam accession number PS00179) at about amino acid residues 48-70 of SEQ ID NO:16.

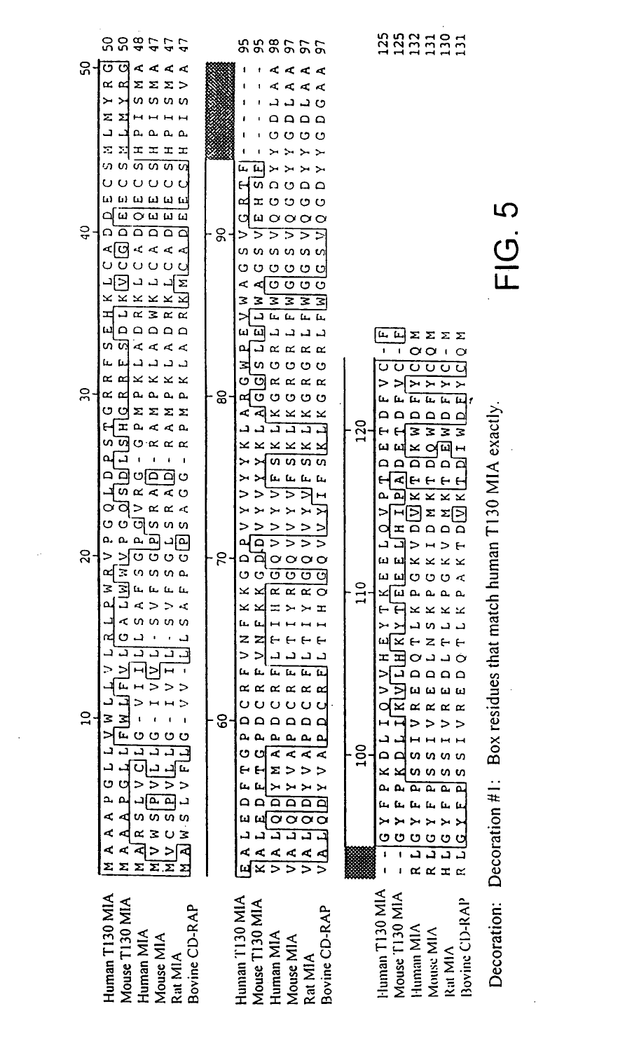

A region of mouse and human TANGO 130 proteins (SEQ ID NO:6 and SEQ ID NOs:12 and 19, respectively) bears some similarity to a MIA homology domain derived by analysis of a comparison between human MIA, rat MIA, mouse MIA and bovine CD-RAP (FIG. 5). The “MIA homology domain” contains a consensus sequence comprising preferably at least about 135 amino acids, more preferably at least about 130, 125, 120, 100, or 50 amino acids. The consensus sequence, derived from the alignment shown in FIG. 5, is as follows:

(SEQ ID NO:13)

M(X)₆L(X)₄₋₅L(X)₁₉₋₂₁K(L/V)C(A/G)DXECS(X)₇ALXD(X)₃
PDCRF(X)₅GXXVYVXXKL(X)₇WXGSV(X)₄₋₁₂GYFP(X)₁₉DXXDFX
CX,

wherein “M” corresponds to the TANGO 130 initiation methionine and “X” represents any amino acid. This consensus sequence also contains 4 conserved cysteines (underlined). The positions of other highly conserved amino acids are indicated with the single letter amino acid code.

The MIA homology domain of TANGO 130 comprises amino acids 1 to 125 of human and mouse TANGO 130 (SEQ ID NOs:12 and 19, and SEQ ID NO:6, respectively). Amino acids 36 through 98 in particular show a high degree of identity between TANGO 130 and MIA/CD-RAP. Also of note is the conservation of four cysteines (underlined above) within the TANGO 130 proteins at amino acid positions 38, 43, 61 and 124 of SEQ ID NO:3, SEQ ID NO:9, and SEQ ID NO:16. The four conserved cysteines are believed to form two intramolecular disulfide bonds and are likely important for maintaining the molecule as an active protein. The positions of these cysteines match those of human, mouse, rat and bovine MIA/CD-RAP.

An approximately 7 kb TANGO 130 mRNA transcript is expressed at a moderate level in mouse liver, testis, heart and skeletal muscle. Lower levels of this transcript were observed in mouse brain. Another TANGO 130 transcript of approximately 1 kb was detected in mouse testis. Additionally, the approximately 7 kb size transcript of TANGO 130 was expressed throughout mouse embryonic development with the highest expression in day 7 embryo.

Mouse and human TANGO 130 are members of a family of molecules (the “TANGO 130 family”) having certain conserved structural and functional features. The term “family” when referring to the protein and nucleic acid molecules of the invention is intended to mean two or more proteins or nucleic acid molecules having a common structural domain and having sufficient amino acid or nucleotide sequence identity as defined herein. Such family members can be naturally occurring and can be from either the same or different species. For example, a family can contain a first protein of human origin and a homologue of that protein of murine origin, as well as a second, distinct protein of human origin and a murine homologue of that protein. Members of a family may also have common functional characteristics.

In one embodiment, a TANGO 130 protein includes a MIA homology domain having at least about 45%, preferably at least about 55%, and more preferably about 65%, 75%, 85%, 95%, or 98% amino acid sequence identity to the MIA homology domain of SEQ ID NO:6, SEQ ID NO:12, or SEQ ID NO:19.

Preferred TANGO 130 polypeptides of the present invention have an amino acid sequence sufficiently identical to the MIA homology domain of SEQ ID NO:6, SEQ ID NO:12, or SEQ ID NO:19. The term “sufficiently identical” is used herein to refer to a first amino acid or nucleotide sequence which contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences which contain a common structural domain having about 45% identity, preferably 55% identity, and more preferably 65%, 75%, 85%, 95%, or 98% identity are defined herein as sufficiently identical.

As used interchangeably herein a “TANGO 130 activity”, “biological activity of TANGO 130” or “functional activity of TANGO 130”, refers to an activity exerted by a TANGO 130 protein, polypeptide or nucleic acid molecule on a TANGO 130 responsive cell as determined in vivo, or in vitro, according to standard techniques. A TANGO 130 activity can be a direct activity, such as an association with or an enzymatic activity on a second protein or an indirect activity, such as a cellular signaling activity mediated by interaction of the TANGO 130 protein with a second protein. In a preferred embodiment, a TANGO 130 activity includes at least one or more of the following activities: (i) the ability to interact with proteins in the TANGO 130 signaling pathway; (ii) the ability to interact with a TANGO 130 receptor; and (iii) the ability to interact with an intracellular target protein. Other activities include: (1) the ability to modulate, e.g., inhibit, cell proliferation (e.g., proliferation of cells of the kidney, liver, heart, testis, immune system, skin, cartilage, skeleton, skeletal muscle and central nervous system), e.g., abnormal cell proliferation, e.g., malignant cell proliferation, e.g., malignant proliferation of epithelial cells (e.g., carcinomas, e.g., melanomas), malignant proliferation of mesenchymal cells (e.g., sarcomas, e.g., chondrosarcomas, glioblastomas); (2) the ability to modulate cell-cell interactions, e.g., cell adhesion, or cell-substrate interactions; (3) the ability to modulate cell migration, e.g., abnormal migration, e.g., metastasis of tumor cells; (4) the ability to modulate cell differentiation (e.g., differentiation of cells of the kidney, liver, heart, testis, immune system, skin, cartilage, skeleton, skeletal muscle and central nervous system); (5) the ability to modulate retinoic acid-mediated functions or activities, e.g., differentiation, e.g., differentiation of cells of the kidney, liver, heart, testis, immune system, skin, cartilage, skeleton, skeletal muscle and central nervous system, cell proliferation, e.g., proliferation of cells of the kidney, liver, heart, testis, immune system, skin, cartilage, skeleton, skeletal muscle and central nervous system or modulation of transcription factor function, e.g., modulation of AP-2 function; and (6) the ability to modulate embryonic cell growth and/or morphogenesis (e.g., of embryonic cells of the kidney, liver, heart, testis, immune system, skin, cartilage, skeleton, skeletal muscle and central nervous system).

TANGO 130 molecules of the invention can be assayed for their ability to modulate growth, e.g., proliferation, or metabolism of cells. Such activities can be detected by, for example, measurement of ³H-thymidine uptake in response to exposure of cells to TANGO 130 molecules (as described in Canadian Patent Application Number 2,167,693 at page 28). Briefly, cells are treated with a TANGO 130 molecule for several days and then pulsed with ³H-thymidine. Radioactivity incorporated into DNA, as measured by, e.g., scintillation counting of the TCA-precipitated DNA, is indicative of DNA synthesis and cellular proliferation.

In addition, TANGO 130 molecules of the invention can be assayed for their ability to modulate tumor cell transforming potential. For example, tumor colony formation can be measured in soft agar (e.g., by transfecting tumor cells with TANGO 130 DNA molecules of the invention, plating in soft agar and monitoring for growth over several weeks). Alternatively, invasiveness of tumor cells can be measured, for example, as the ability to modulate tumor chemotaxis to an attractant through a Costar Boyden chamber (see Canadian Patent Application Number 2,167,693 at page 30; Albini et al. (1987) Cancer Res. 47:3239). The ability of TANGO 130 molecules to modulate invasiveness can also be measured in vivo, for example, by monitoring tumor growth in mice injected with cells, e.g., transformed cells, that have been modified by transfection with a TANGO 130 DNA molecule to express a TANGO 130 protein. Thus, TANGO 130 expression may alter the ability of tumorigenic cells to invade tissues and form tumors in mice.

The ability of TANGO 130 molecules of the invention to modulate cellular adhesiveness or cellular morphology can also be tested. In one embodiment, cells are examined microscopically before and after treatment with TANGO 130 molecules of the invention. TANGO 130 treatment of eukaryotic cells, e.g., metastatic tumor cells or transformed cell lines, may result in observable changes in cell morphology and adhesion, e.g., from flat-shaped and tightly adherent to rounded and less adherent.

The ability of TANGO 130 molecules of the invention to modulate cell differentiation and proliferation includes the use of TANGO 130 molecules to modulate the regenerative capacity of tissues, e.g., of kidney, liver, heart, testis, skeleton, cartilage, skin, brain, immune cells or skeletal muscle. A variety of assays are known in the art for examining regenerative potential of tissues. For example, an ischemic/reperfusion model can be used in kidney, in which the kidney's capacity for renewal is measured following injury (see Ichimura et al. (1998) J. Biol. Chem. 273:4135). Tissue injury and regeneration, with and without pre-treatment with TANGO 130 molecules, can be monitored by immunohistochemistry using markers for regeneration, e.g., vimentin detected using anti-vimentin monoclonal antibodies in sectioned tissue.

Accordingly, another embodiment of the invention features isolated TANGO 130 proteins and polypeptides having a TANGO 130 activity.

Additionally, TANGO 130 molecules of the invention are involved in disorders which affect both tissues in which they are normally expressed and tissues in which they are normally not expressed. For example, TANGO 130 is involved in modulating proliferation, migration, morphology, differentiation, and/or function of cells of tissues is which is it expressed. In addition to the disorders listed herein, TANGO 130 polypeptides, nucleic acids, and modulators thereof of the invention can also be used to treat at least the following disorders:

TANGO 130 polypeptides, nucleic acids, and modulators thereof can be used to treat pancreatic disorders, such as pancreatitis (e.g., acute hemorrhagic pancreatitis and chronic pancreatitis), pancreatic cysts (e.g., congenital cysts, pseudocysts, and benign or malignant neoplastic cysts), pancreatic tumors (e.g., pancreatic carcinoma and adenoma), diabetes mellitus (e.g., insulin- and non-insulin-dependent types, impaired glucose tolerance, and gestational diabetes), or islet cell tumors (e.g., insulinomas, adenomas, Zollinger-Ellison syndrome, glucagonomas, and somatostatinoma).

In another example, TANGO 130 polypeptides, nucleic acids, and modulators thereof can be used to treat cardiovascular disorders, such as ischemic heart disease (e.g., angina pectoris, myocardial infarction, and chronic ischemic heart disease), hypertensive heart disease, pulmonary heart disease, valvular heart disease (e.g., rheumatic fever and rheumatic heart disease, endocarditis, mitral valve prolapse, and aortic valve stenosis), congenital heart disease (e.g., valvular and vascular obstructive lesions, atrial or ventricular septal defect, and patent ductus arteriosus), or myocardial disease (e.g., myocarditis, congestive cardiomyopathy, and hypertrophic cariomyopathy).

In another example, TANGO 130 polypeptides, nucleic acids, and modulators thereof can be used to treat hepatic (liver) disorders, such as jaundice, hepatic failure, hereditary hyperbiliruinemias (e.g., Gilbert's syndrome, Crigler-Naijar syndromes and Dubin-Johnson and Rotor's syndromes), hepatic circulatory disorders (e.g., hepatic vein thrombosis and portal vein obstruction and thrombosis), hepatitis (e.g., chronic active hepatitis, acute viral hepatitis, and toxic and drug-induced hepatitis), cirrhosis (e.g., alcoholic cirrhosis, biliary cirrhosis, and hemochromatosis), or malignant tumors (e.g., primary carcinoma, hepatoma, hepatoblastoma, liver cysts, and angiosarcoma).

In another example, TANGO 130 polypeptides, nucleic acids, and modulators thereof can be used to treat renal (kidney) disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, polycystic kidney disease, neoplasia, sickle cell disease, and chronic inflammatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, gout, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

In another example, TANGO 130 polypeptides, nucleic acids, and modulators thereof can be used to treat testicular disorders, such as unilateral testicular enlargement (e.g., nontuberculous, granulomatous orchitis); inflammatory diseases resulting in testicular dysfunction (e.g., gonorrhea and mumps); cryptorchidism; sperm cell disorders (e.g., immotile cilia syndrome and germinal cell aplasia); acquired testicular defects (e.g., viral orchitis); and tumors (e.g., germ cell tumors, interstitial cell tumors, androblastoma, testicular lymphoma and adenomatoid tumors).

In another example, TANGO 130 polypeptides, nucleic acids, and modulators thereof can be used to treat disorders of skeletal muscle, such as muscular dystrophy (e.g., Duchenne Muscular Dystrophy, Becker Muscular Dystrophy, Emery-Dreifuss Muscular Dystrophy, Limb-Girdle Muscular Dystrophy, Facioscapulohumeral Muscular Dystrophy, Myotonic Dystrophy, Oculopharyngeal Muscular Dystrophy, Distal Muscular Dystrophy, and Congenital Muscular Dystrophy), motor neuron diseases (e.g., Amyotrophic Lateral Sclerosis, Infantile Progressive Spinal Muscular Atrophy, Intermediate Spinal Muscular Atrophy, Spinal Bulbar Muscular Atrophy, and Adult Spinal Muscular Atrophy), myopathies (e.g., inflammatory myopathies (e.g., Dermatomyositis and Polymyositis), Myotonia Congenita, Paramyotonia Congenita, Central Core Disease, Nemaline Myopathy, Myotubular Myopathy, and Periodic Paralysis), and metabolic diseases of muscle (e.g., Phosphorylase Deficiency, Acid Maltase Deficiency, Phosphofructokinase Deficiency, Debrancher Enzyme Deficiency, Mitochondrial Myopathy, Carnitine Deficiency, Carnitine Palmityl Transferase Deficiency, Phosphoglycerate Kinase Deficiency, Phosphoglycerate Mutase Deficiency, Lactate Dehydrogenase Deficiency, and Myoadenylate Deaminase Deficiency).

In another example, TANGO 130 polypeptides, nucleic acids, and modulators thereof can be used to treat brain and CNS related disorders. Such brain and CNS related disorders include but are not limited to bacterial and viral meningitis, cerebral toxoplasmosis, brain cancers (e.g., metastatic carcinoma of the brain, glioblastoma, lymphoma, astrocytoma, acoustic neuroma), hydrocephalus, and encephalitis.

In another example, TANGO 130 polypeptides, nucleic acids, and modulators thereof can be used to treat spleen disorders, including e.g., splenic lymphoma and/or splenomegaly, and/or phagocytotic disorders, e.g., those inhibiting macrophage engulfment of bacteria and viruses in the bloodstream.

Various aspects of the invention are described in further detail in the following subsections.

I. Isolated Nucleic Acid Molecules

One aspect of the invention pertains to isolated nucleic acid molecules that encode TANGO 130 proteins or biologically active portions thereof, as well as nucleic acid molecules sufficient for use as hybridization probes to identify TANGO 130-encoding nucleic acids (e.g., TANGO 130 mRNA) and fragments for use as PCR primers for the amplification or mutation of TANGO 130 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid. Preferably, an “isolated” nucleic acid is free of sequences (preferably protein encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated TANGO 130 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, SEQ ID NO:2, SEQ ID NO:7, SEQ ID NO:8, SEQ ID NO:14, SEQ ID NO:15, the cDNA of ATCC 98823, the cDNA of ATCC 98844, the cDNA of ATCC 98845, or a complement of any of these nucleotide sequences, can be isolated using standard molecular biology techniques and the sequence information provided herein. Using all or a portion of the coding or non-coding nucleic acid sequences of SEQ ID NO:1, SEQ ID NO:7, SEQ ID NO:14, the cDNA of ATCC 98823, the cDNA of ATCC 98844, or the cDNA of ATCC 98845 as a hybridization probe, TANGO 130 nucleic acid molecules can be isolated using standard hybridization and cloning techniques (e.g., as described in Sambrook et al., eds., Molecular Cloning: A Laboratory Manual, 2nd ed., Cold Spring Harbor Laboratory, Cold Spring Harbor Laboratory Press, Cold Spring Harbor, N.Y., 1989).

A nucleic acid molecule of the invention can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to TANGO 130 nucleotide sequences can be prepared by standard synthetic techniques, e.g., using an automated DNA synthesizer.

In another preferred embodiment, an isolated nucleic acid molecule of the invention comprises a nucleic acid molecule which is a complement of the nucleotide sequence shown in SEQ ID NO:1, SEQ ID NO:7, SEQ ID NO:14, the cDNA of ATCC 98823, the cDNA of ATCC 98844, the cDNA of ATCC 98845, or a portion thereof. A nucleic acid molecule which is complementary to a given nucleotide sequence is one which is sufficiently complementary to the given nucleotide sequence that it can hybridize to the given nucleotide sequence to thereby form a stable duplex.

Moreover, the nucleic acid molecule of the invention can comprise only a portion of a nucleic acid sequence encoding TANGO 130, for example, a fragment which can be used as a probe or primer or a fragment encoding a biologically active portion of TANGO 130. The nucleotide sequences determined from the cloning of the mouse and human TANGO 130 genes allow for the generation of probes and primers designed for use in identifying and/or cloning TANGO 130 homologues in other cell types, e.g., from other tissues, as well as TANGO 130 homologues from other mammals. The probe/primer typically comprises substantially purified oligonucleotide. The oligonucleotide typically comprises a region of nucleotide sequence that hybridizes under stringent conditions to at least about 12, preferably about 25, more preferably about 50, 75, 100, 125, 150, 175, 200, 250, 300, 350, 400, 450, 500, 550, 600, 650, 700, 750, 800, 850, 900, 950, or 1000 consecutive nucleotides of the sense or anti-sense sequence of SEQ ID NO:1, SEQ ID NO:7, SEQ ID NO:14, the cDNA of ATCC 98823, the cDNA of ATCC 98844, or the cDNA of ATCC 98845; or of a naturally occurring mutant of SEQ ID NO:1, SEQ ID NO:7, SEQ ID NO:14, the cDNA of ATCC 98823, the cDNA of ATCC 98844, or the cDNA of ATCC 98845.

Probes based on the human TANGO 130 nucleotide sequence can be used to detect transcripts or genomic sequences encoding the same or identical proteins. The probe comprises a label group attached thereto, e.g., a radioisotope, a fluorescent compound, an enzyme, or an enzyme co-factor. Such probes can be used as part of a diagnostic test kit for identifying cells or tissues which mis-express a TANGO 130 protein, such as by measuring levels of a TANGO 130-encoding nucleic acid in a sample of cells from a subject, e.g., detecting TANGO 130 mRNA levels or determining whether a genomic TANGO 130 gene has been mutated or deleted.

A nucleic acid fragment encoding a “biologically active portion of TANGO 130” can be prepared by isolating a portion of SEQ ID NO:1, SEQ ID NO:7, the nucleotide sequence of the cDNA of ATCC 98823, the nucleotide sequence of the cDNA of ATCC 98844, or the nucleotide sequence of the cDNA of ATCC 98845, which encodes a polypeptide having a TANGO 130 biological activity, expressing the encoded portion of TANGO 130 protein (e.g., by recombinant expression in vitro) and assessing the activity of the encoded portion of TANGO 130. For example, a nucleic acid fragment encoding a biologically active portion of TANGO 130 includes a MIA homology domain, e.g., SEQ ID NO:6, SEQ ID NO:12, or SEQ ID NO:19.

The invention further encompasses nucleic acid molecules that differ from the nucleotide sequence of SEQ ID NO:1, SEQ ID NO:2, SEQ ID NO:7, SEQ ID NO:8, SEQ ID NO:14, SEQ ID NO:15, the cDNA of ATCC 98823, the cDNA of ATCC 98844, or the cDNA of ATCC 98845 due to degeneracy of the genetic code and thus encode the same TANGO 130 protein as that encoded by the nucleotide sequence shown in SEQ ID NO:1, SEQ ID NO:2, SEQ ID NO:7, SEQ ID NO:8, SEQ ID NO:14, SEQ ID NO:15, the cDNA of ATCC 98823, the cDNA of ATCC 98844, or the cDNA of ATCC 98845.

In addition to the mouse and human TANGO 130 nucleotide sequences shown in SEQ ID NO:1, SEQ ID NO:7, SEQ ID NO:14, the cDNA of ATCC 98823, the cDNA of 98844, or the cDNA of ATCC 98845, it will be appreciated by those skilled in the art that DNA sequence polymorphisms that lead to changes in the amino acid sequences of TANGO 130 may exist within a population (e.g., the human population). Such genetic polymorphism in the TANGO 130 gene may exist among individuals within a population due to natural allelic variation. An allele is one of a group of genes which occur alternatively at a given genetic locus. As used herein, the terms “gene” and “recombinant gene” refer to nucleic acid molecules comprising an open reading frame encoding a TANGO 130 protein, preferably a mammalian TANGO 130 protein.

As used herein, the phrase “allelic variant” refers to a nucleotide sequence which occurs at a TANGO 130 locus or to a polypeptide encoded by the nucleotide sequence. For example, TANGO 130 maps to murine chromosome 5, between markers D5Mit195 and D5Mit15, an area which is syntenic to human 7q, 7p, 18p1, 4p1, 14q. Such natural allelic variations can typically result in 1-5% variance in the nucleotide sequence of the TANGO 130 gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations in TANGO 130 that are the result of natural allelic variation and that do not alter the functional activity of TANGO 130 are intended to be within the scope of the invention.

Moreover, nucleic acid molecules encoding TANGO 130 proteins from other species (TANGO 130 homologues), which have a nucleotide sequence which differs from that of a human TANGO 130, are intended to be within the scope of the invention. Nucleic acid molecules corresponding to natural allelic variants and homologues of the TANGO 130 cDNA of the invention can be isolated based on their identity to the human TANGO 130 nucleic acids disclosed herein using the mouse or human cDNAs, or a portion thereof, as a hybridization probe according to standard hybridization techniques under stringent hybridization conditions. Similarly, alternate forms of TANGO 130, e.g., membrane-bound forms, can be isolated based on their ability to bind a TANGO 130 hybridization probe.

Accordingly, in another embodiment, an isolated nucleic acid molecule of the invention is at least 500 (550, 600, 650, 700, 800, 900, 1000, 1250, 1500, 1750, 2000, 2250, 2500, 2750, 3000, 3250, 3750, 4000, 4250, 4500, 4750, 5000, 5250, 5500, 5750, 6000, 6250, 6500, 6750, 7000, 7250, 7500, 7750, or 8000) nucleotides in length and hybridizes under stringent conditions to the nucleic acid molecule comprising the nucleotide sequence, preferably the coding sequence, of SEQ ID NO:1, SEQ ID NO:7, the cDNA of ATCC 98823, the cDNA of ATCC 98844, the cDNA of ATCC 98845, or a complement thereof.

As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45(C, followed by one or more washes in 0.2× SSC, 0.1% SDS at 50-65(C. Preferably, an isolated nucleic acid molecule of the invention that hybridizes under stringent conditions to the sequence of SEQ ID NO:1, SEQ ID NO:2, SEQ ID NO:7, SEQ ID NO:8, SEQ ID NO:14, SEQ ID NO:15, the cDNA of ATCC 98823, the cDNA of ATCC 98844, the cDNA of ATCC 98845, or the complement thereof, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the TANGO 130 sequence that may exist in the population, the skilled artisan will further appreciate that changes can be introduced by mutation into the nucleotide sequence of SEQ ID NO:1, SEQ ID NO:2, SEQ ID NO:7, SEQ ID NO:8, SEQ ID NO:14, SEQ ID NO:15, the cDNA of ATCC 98823, the cDNA of ATCC 98844, or the cDNA of ATCC 98845, thereby leading to changes in the amino acid sequence of the encoded TANGO 130 protein, without altering the biological activity of the TANGO 130 protein. For example, one can make nucleotide substitutions leading to amino acid substitutions at “non-essential” amino acid residues. A “non-essential” amino acid residue is a residue that can be altered from the wild-type sequence of TANGO 130 (e.g., the sequences of SEQ ID NO:3, SEQ ID NO:9, or SEQ ID NO:16) without altering the biological activity, whereas an “essential” amino acid residue is required for biological activity. For example, amino acid residues that are not conserved or only semi-conserved among TANGO 130 of various species may be non-essential for activity and thus would be likely targets for alteration. Alternatively, amino acid residues that are conserved among the TANGO 130 proteins of various species may be essential for activity and thus would not be likely targets for alteration.

For example, preferred TANGO 130 proteins of the present invention contain at least one MIA homology domain, as described herein (see, e.g., FIG. 5). Within this domain are a number of amino acid residues which are conserved between human and mouse MIA, bovine CD-RAP, and human and mouse TANGO 130. For example, at approximately amino acids 42 to 44 of SEQ ID NO:3, SEQ ID NO:9, and SEQ ID NO:16, the sequence glutamate-cysteine-serine (ECS) is conserved; at approximately amino acids 59 to 63 of SEQ ID NO:3, SEQ ID NO:9, and SEQ ID NO:16, the sequence proline-aspartate-cysteine-arginine-phenylalanine (PDCRF) is conserved; and at approximately amino acids 95 to 98 of SEQ ID NO:3, SEQ ID NO:9, and SEQ ID NO:16, the sequence glycine-tyrosine-phenylalanine-proline (GYFP) is conserved. These conserved amino acids, for example, are likely to be essential for activity and thus would not be likely targets for alteration. Additionally, preferred TANGO 130 proteins of the present invention contain approximately four cysteine residues in positions corresponding to those shown in FIG. 5. Thus, these cysteines are likely to be essential and would not be likely targets for alteration.

Accordingly, another aspect of the invention pertains to nucleic acid molecules encoding TANGO 130 proteins that contain changes in amino acid residues that are not essential for activity. Such TANGO 130 proteins differ in amino acid sequence from SEQ ID NO:3, SEQ ID NO:9, and SEQ ID NO:16 yet retain biological activity. In one embodiment, the isolated nucleic acid molecule includes a nucleotide sequence encoding a protein that includes an amino acid sequence that is at least about 45% identical, 55%, 65%, 75%, 85%, 95%, or 98% identical to the amino acid sequences of SEQ ID NO:3, SEQ ID NO:9, or SEQ ID NO:16.

An isolated nucleic acid molecule encoding a TANGO 130 protein having a sequence which differs from that of SEQ ID NO:3, SEQ ID NO:9, or SEQ ID NO:16 can be created by introducing one or more nucleotide substitutions, additions or deletions into the nucleotide sequence of SEQ ID NO:1, SEQ ID NO:2, SEQ ID NO:7, SEQ ID NO:8, SEQ ID NO:14, SEQ ID NO:15, the cDNA of ATCC 98823, the cDNA of ATCC 98844, or the cDNA of ATCC 98845 such that one or more amino acid substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in TANGO 130 is preferably replaced with another amino acid residue from the same side chain family. Alternatively, mutations can be introduced randomly along all or part of a TANGO 130 coding sequence, such as by saturation mutagenesis, and the resultant mutants can be screened for TANGO 130 biological activity to identify mutants that retain activity. Following mutagenesis, the encoded protein can be expressed recombinantly and the activity of the protein can be determined. In a preferred embodiment, a mutant TANGO 130 protein can be assayed for the activities described herein.

In one embodiment, a mutant polypeptide that is a variant of a polypeptide of the invention can be assayed for: (1) the ability to form protein:protein interactions with a polypeptide of the invention; (2) the ability to bind a ligand of a polypeptide of the invention; (3) the ability to bind with a modulator or substrate of a polypeptide of the invention; (4) the ability to modulate a physiological activity of a polypeptide of the invention, such as one of those disclosed herein; or (5) the ability to catalyze a reaction catalyzed by a polypeptide of the invention.

The present invention encompasses antisense nucleic acid molecules, i.e., molecules which are complementary to a sense nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire TANGO 130 coding strand, or to only a portion thereof, e.g., all or part of the protein coding region (or open reading frame). An antisense nucleic acid molecule can be antisense to a noncoding region of the coding strand of a nucleotide sequence encoding TANGO 130. The noncoding regions (“5′ and 3′ untranslated regions”) are the 5′ and 3′ sequences which flank the coding region and are not translated into amino acids.

Given the coding strand sequences encoding TANGO 130 disclosed herein (e.g., SEQ ID NO:1, SEQ ID NO:2, SEQ ID NO:7, SEQ ID NO:8, SEQ ID NO:14, or SEQ ID NO:15), antisense nucleic acids of the invention can be designed according to the rules of Watson and Crick base pairing. The antisense nucleic acid molecule can be complementary to the entire coding region of TANGO 130 mRNA, but more preferably is an oligonucleotide which is antisense to only a portion of the coding or noncoding region of TANGO 130 mRNA. For example, the antisense oligonucleotide can be complementary to the region surrounding the translation start site of TANGO 130 mRNA, e.g., an oligonucleotide having the sequence corresponding to nucleotides 18 to 36, nucleotides 21 to 50, or nucleotides 20 to 66 of SEQ ID NO:1, or corresponding to nucleotides 31 to 49, nucleotides 30 to 76, or nucleotides 25 to 56 of SEQ ID NO:7 or SEQ ID NO:14. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a TANGO 130 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625). The antisense nucleic acid molecule can also comprise a 2′-o-methylribonucleotide (Inoue et al. (1987) Nucleic Acids Res. 15:6131) or a chimeric RNA-DNA analogue (Inoue et al. (1987) FEBS Lett. 215:327).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585)) can be used to catalytically cleave TANGO 130 mRNA transcripts to thereby inhibit translation of TANGO 130 mRNA. A ribozyme having specificity for a TANGO 130-encoding nucleic acid can be designed based upon the nucleotide sequence of a TANGO 130 cDNA disclosed herein (e.g., SEQ ID NO:1, SEQ ID NO:2, SEQ ID NO:7, or SEQ ID NO:8). For example, a derivative of a Tetrahymena L-19 IVS RNA can be constructed in which the nucleotide sequence of the active site is complementary to the nucleotide sequence to be cleaved in a TANGO 130-encoding mRNA. See, e.g., Cech et al. U.S. Pat. No. 4,987,071; and Cech et al. U.S. Pat. No. 5,116,742. Alternatively, TANGO 130 mRNA can be used to select a catalytic RNA having a specific ribonuclease activity from a pool of RNA molecules. See, e.g., Bartel and Szostak (1993) Science 261:1411.

The invention also encompasses nucleic acid molecules which form triple helical structures. For example, TANGO 130 gene expression can be inhibited by targeting nucleotide sequences complementary to the regulatory region of the TANGO 130 (e.g., the TANGO 130 promoter and/or enhancers) to form triple helical structures that prevent transcription of the TANGO 130 gene in target cells. See generally Helene (1991) Anticancer Drug Des. 6(6):569; Helene (1992) Ann. N.Y. Acad. Sci. 660:27; Maher (1992) Bioassays 14(12):807.

In preferred embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4:5). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) supra; Perry-O'Keefe et al. (1996) Proc. Natl. Acad. Sci. USA 93:14670.

PNAs of TANGO 130 can be used in therapeutic and diagnostic applications. For example, PNAs can be used as antisense or antigene agents for sequence-specific modulation of gene expression by, e.g., inducing transcription or translation arrest or inhibiting replication. PNAs of TANGO 130 can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra) or as probes or primers for DNA sequence and hybridization (Hyrup (1996), supra; Perry-O'Keefe et al. (1996), supra).

In another embodiment, PNAs of TANGO 130 can be modified, e.g., to enhance their stability, specificity or cellular uptake, by attaching lipophilic or other helper groups to PNA, by the formation of PNA-DNA chimeras, or by the use of liposomes or other techniques of drug delivery known in the art. The synthesis of PNA-DNA chimeras can be performed as described in Hyrup (1996) supra; Finn et al. (1996) Nucleic Acids Res. 24(17):3357; Mag et al. (1989) Nucleic Acids Res. 17:5973; and Peterser et al. (1975) Bioorganic Med. Chem. Lett. 5:1119.

II. Isolated TANGO 130 Proteins and Anti-TANGO 130 Antibodies

One aspect of the invention pertains to isolated TANGO 130 proteins, and biologically active portions thereof, as well as polypeptide fragments suitable for use as immunogens to raise anti-TANGO 130 antibodies. In one embodiment, native TANGO 130 proteins can be isolated from cells or tissue sources by an appropriate purification scheme using standard protein purification techniques. In another embodiment, TANGO 130 proteins are produced by recombinant DNA techniques. Alternative to recombinant expression, a TANGO 130 protein or polypeptide can be synthesized chemically using standard peptide synthesis techniques.

An “isolated” or “purified” protein or biologically active portion thereof is substantially free of cellular material or other contaminating proteins from the cell or tissue source from which the TANGO 130 protein is derived, or substantially free of chemical precursors or other chemicals when chemically synthesized. The language “substantially free of cellular material” includes preparations of TANGO 130 protein in which the protein is separated from cellular components of the cells from which it is isolated or recombinantly produced. Thus, TANGO 130 protein that is substantially free of cellular material includes preparations of TANGO 130 protein having less than about 30%, 20%, 10%, or 5% (by dry weight) of non-TANGO 130 protein (also referred to herein as a “contaminating protein”). When the TANGO 130 protein or biologically active portion thereof is recombinantly produced, it is also preferably substantially free of culture medium, i.e., culture medium represents less than about 20%, 10%, or 5% of the volume of the protein preparation. When TANGO 130 protein is produced by chemical synthesis, it is preferably substantially free of chemical precursors or other chemicals, i.e., it is separated from chemical precursors or other chemicals which are involved in the synthesis of the protein. Accordingly such preparations of TANGO 130 protein have less than about 30%, 20%, 10%, 5% (by dry weight) of chemical precursors or non-TANGO 130 chemicals.

Biologically active portions of a TANGO 130 protein include peptides comprising amino acid sequences sufficiently identical to or derived from the amino acid sequence of the TANGO 130 protein (e.g., the amino acid sequence shown in SEQ ID NO:3, SEQ ID NO:9, or SEQ ID NO:16), which include fewer amino acids than the full length TANGO 130 proteins, and exhibit at least one activity of a TANGO 130 protein. Typically, biologically active portions comprise a domain or motif with at least one activity of the TANGO 130 protein. A biologically active portion of a TANGO 130 protein can be a polypeptide which is, for example, 10, 25, 50, 100 or more amino acids in length. Preferred biologically active polypeptides include one or more identified TANGO 130 structural domains, e.g., the MIA homology domain (e.g., SEQ ID NO:6, SEQ ID NO:12, or SEQ ID NO:19).

Moreover, other biologically active portions, in which other regions of the protein are deleted, can be prepared by recombinant techniques and evaluated for one or more of the functional activities of a native TANGO 130 protein.

Preferred TANGO 130 protein has the amino acid sequence of SEQ ID NO:3, SEQ ID NO:9, or SEQ ID NO:16. Other useful TANGO 130 proteins are substantially identical to SEQ ID NO:3, SEQ ID NO:9, or SEQ ID NO:16 and retain the functional activity of the protein of SEQ ID NO:3, SEQ ID NO:9, or SEQ ID NO:16 yet differ in amino acid sequence due to natural allelic variation or mutagenesis. For example, such TANGO 130 proteins and polypeptides possess at least one biological activity described herein. Accordingly, a useful TANGO 130 protein is a protein which includes an amino acid sequence at least about 45%, preferably 55%, 65%, 75%, 85%, 95%, or 99% identical to the amino acid sequence of SEQ ID NO:3, SEQ ID NO:9, or SEQ ID NO:16 and retains the functional activity of the TANGO 130 proteins of SEQ ID NO:3, SEQ ID NO:9, or SEQ ID NO:16. In other instances, the TANGO 130 protein is a protein having an amino acid sequence 55%, 65%, 75%, 85%, 95%, or 98% identical to the TANGO 130 MIA homology domain (SEQ ID NO:6 or SEQ ID NO:12). In a preferred embodiment, the TANGO 130 protein retains a functional activity of the TANGO 130 protein of SEQ ID NO:3, SEQ ID NO:9, or SEQ ID NO:16.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions {e.g., overlapping positions}×100). In one embodiment, the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov.

Another preferred, non-limiting example of a mathematical algorithm utilized for the comparison of sequences is the local homology algorithm of Smith and Waterman (Advances in Applied Mathematics 2: 482-489 (1981)). Such an algorithm is incorporated into the BestFit program, which is part of the Wisconsin™ package, and is used to find the best segment of similarity between two sequences. BestFit reads a scoring matrix that contains values for every possible GCG symbol match. The program uses these values to construct a path matrix that represents the entire surface of comparison with a score at every position for the best possible alignment to that point. The quality score for the best alignment to any point is equal to the sum of the scoring matrix values of the matches in that alignment, less the gap creation penalty multiplied by the number of gaps in that alignment, less the gap extension penalty multiplied by the total length of all gaps in that alignment. The gap creation and gap extension penalties are set by the user. If the best path to any point has a negative value, a zero is put in that position.

After the path matrix is complete, the highest value on the surface of comparison represents the end of the best region of similarity between the sequences. The best path from this highest value backwards to the point where the values revert to zero is the alignment shown by BestFit. This alignment is the best segment of similarity between the two sequences. Further documentation can be found at http://ir.ucdavis.edu/GCGhelp/bestfit.html#algorithm.

Additional algorithms for sequence analysis are known in the art and include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. For a further description of FASTA parameters, see http://bioweb.pasteur.fr/docs/man/man/fasta.1.html#sect2, the contents of which are incorporated herein by reference.

The percent identity between two sequences can be determined using techniques similar to those described above, with or without allowing gaps. In calculating percent identity, only exact matches are counted.

The invention also provides TANGO 130 chimeric or fusion proteins. As used herein, a TANGO 130 “chimeric protein” or “fusion protein” comprises a TANGO 130 polypeptide operably linked to a non-TANGO 130 polypeptide. A “TANGO 130 polypeptide” refers to a polypeptide having an amino acid sequence corresponding to TANGO 130, whereas a “non-TANGO 130 polypeptide” refers to a polypeptide having an amino acid sequence corresponding to a protein which is not substantially identical to the TANGO 130 protein, e.g., a protein which is different from the TANGO 130 protein and which is derived from the same or a different organism. Within a TANGO 130 fusion protein the TANGO 130 polypeptide can correspond to all or a portion of a TANGO 130 protein, preferably at least one biologically active portion of a TANGO 130 protein. Within the fusion protein, the term “operably linked” is intended to indicate that the TANGO 130 polypeptide and the non-TANGO 130 polypeptide are fused in-frame to each other. The non-TANGO 130 polypeptide can be fused to the N-terminus or C-terminus of the TANGO 130 polypeptide.

One useful fusion protein is a GST-TANGO 130 fusion protein in which the TANGO 130 sequences are fused to the C-terminus of the GST sequences. Such fusion proteins can facilitate the purification of recombinant TANGO 130.

In another embodiment, the fusion protein is a TANGO 130 protein containing a heterologous signal sequence at its N-terminus. For example, the native TANGO 130 signal sequence (i.e., about amino acids 1 to 24 of SEQ ID NO:3 or about amino acids 1 to 23 of SEQ ID NO:9) can be removed and replaced with a signal sequence from another protein. In certain host cells (e.g., mammalian host cells), expression and/or secretion of TANGO 130 can be increased through use of a heterologous signal sequence. For example, the gp67 secretory sequence of the baculovirus envelope protein can be used as a heterologous signal sequence (Current Protocols in Molecular Biology, Ausubel et al., eds. (1992) John Wiley & Sons). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech, Piscataway, N.J.).

In yet another embodiment, the fusion protein is a TANGO 130-immunoglobulin fusion protein in which all or part of a polypeptide of the invention is fused with sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction can be useful therapeutically, both for treating proliferative and differentiative disorders and for modulating (e.g., promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands. The immunoglobulin fusion protein can, for example, comprise a portion of a polypeptide of the invention fused with the amino-terminus or the carboxyl-terminus of an immunoglobulin constant region, as disclosed in U.S. Pat. No. 5,714,147, U.S. Pat. No. 5,116,964, U.S. Pat. No. 5,514,582, and U.S. Pat. No. 5,455,165.

Preferably, a TANGO 130 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Ausubel et al., supra). Moreover, many expression vectors are commercially available that already encode a fusion moiety (e.g., a GST polypeptide). An TANGO 130-encoding nucleic acid can be cloned into such an expression vector such that the fusion moiety is linked in-frame to the TANGO 130 protein.

A signal sequence of the invention (e.g., a signal sequence in any of SEQ ID NOs:5, 11, and 18) can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having signal sequences, as well as to the signal sequences themselves and to the polypeptides in the absence of the signal sequence (i.e., the cleavage products; SEQ ID NO:4, SEQ ID NO:10, and SEQ ID NO:17). The location (amino acid position) of the signal sequences of the molecules of the invention are shown in the Figures. In one embodiment, a nucleic acid sequence encoding a signal sequence of the invention can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

In another embodiment, the signal sequences of the present invention can be used to identify regulatory sequences, e.g., promoters, enhancers, repressors. Since signal sequences are the most amino-terminal sequences of a peptide, it is expected that the nucleic acids which flank the signal sequence on its amino-terminal side will be regulatory sequences which affect transcription. Thus, a nucleotide sequence which encodes all or a portion of a signal sequence can be used as a probe to identify and isolate signal sequences and their flanking regions, and these flanking regions can be used to identify regulatory elements therein.

The present invention also pertains to variants of the TANGO 130 proteins (i.e., proteins having a sequence which differs from that of the TANGO 130 amino acid sequence). Such variants can function as either TANGO 130 agonists (mimetics) or as TANGO 130 antagonists. Variants of the TANGO 130 protein can be generated by mutagenesis, e.g., discrete point mutation or truncation of the TANGO 130 protein. An agonist of the TANGO 130 protein can retain substantially the same, or a subset, of the biological activities of the naturally occurring form of the TANGO 130 protein. An antagonist of the TANGO 130 protein can inhibit one or more of the activities of the naturally occurring form of the TANGO 130 protein by, for example, competitively binding to a downstream or upstream member of a cellular signaling cascade which includes the TANGO 130 protein. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the TANGO 130 proteins.

Variants of the TANGO 130 protein which function as either TANGO 130 agonists (mimetics) or as TANGO 130 antagonists can be identified by screening combinatorial libraries of mutants, e.g., truncation mutants, of the TANGO 130 protein for TANGO 130 protein agonist or antagonist activity. In one embodiment, a variegated library of TANGO 130 variants is generated by combinatorial mutagenesis at the nucleic acid level and is encoded by a variegated gene library. A variegated library of TANGO 130 variants can be produced by, for example, enzymatically ligating a mixture of synthetic oligonucleotides into gene sequences such that a degenerate set of potential TANGO 130 sequences is expressible as individual polypeptides, or alternatively, as a set of larger fusion proteins (e.g., for phage display) containing the set of TANGO 130 sequences therein. There are a variety of methods which can be used to produce libraries of potential TANGO 130 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential TANGO 130 sequences. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of the TANGO 130 protein coding sequence can be used to generate a variegated population of TANGO 130 fragments for screening and subsequent selection of variants of a TANGO 130 protein. In one embodiment, a library of coding sequence fragments can be generated by treating a double stranded PCR fragment of a TANGO 130 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with SI nuclease, and ligating the resulting fragment library into an expression vector. By this method, an expression library can be derived which encodes N-terminal and internal fragments of various sizes of the TANGO 130 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of TANGO 130 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify TANGO 130 variants (Arkin and Yourvan (1992) Proc. Natl. Acad. Sci. USA 89:7811; Delgrave et al. (1993) Protein Engineering 6:327).

An isolated TANGO 130 protein, or a portion or fragment thereof, can be used as an immunogen to generate antibodies that bind TANGO 130 using standard techniques for polyclonal and monoclonal antibody preparation. The full-length TANGO 130 protein can be used or, alternatively, the invention provides antigenic peptide fragments of TANGO 130 for use as immunogens. The antigenic peptide of TANGO 130 comprises at least 8 (preferably 10, 15, 20, or 30) amino acid residues of the amino acid sequence shown in SEQ ID NO:3, SEQ ID NO:9, or SEQ ID NO:16 and encompasses an epitope of TANGO 130 such that an antibody raised against the peptide forms a specific immune complex with TANGO 130.

Preferred epitopes encompassed by the antigenic peptide are regions of TANGO 130 that are located on the surface of the protein, e.g., hydrophilic regions. A hydrophobicity analysis of the mouse TANGO 130 protein sequence indicates that the regions between, e.g., amino acids 152 and 215, between amino acids 391 and 436, and between amino acids 570 and 622 of SEQ ID NO:3 are particularly hydrophilic and, therefore, are likely to encode surface residues useful for targeting antibody production. Likewise, hydrophobicity analysis of the human TANGO 130 protein sequences indicates that the regions between, e.g., amino acids 159 and 224, between amino acids 242 and 260, and between amino acids 335 and 363 of SEQ ID NO:9 and SEQ ID NO:16 are particularly hydrophilic and, therefore, are likely to encode surface residues useful for targeting antibody production.

A TANGO 130 immunogen typically is used to prepare antibodies by immunizing a suitable subject, (e.g., rabbit, goat, mouse or other mammal) with the immunogen. An appropriate immunogenic preparation can contain, for example, recombinantly expressed TANGO 130 protein or a chemically synthesized TANGO 130 polypeptide. The preparation can further include an adjuvant, such as Freund's complete or incomplete adjuvant, or similar immunostimulatory agent. Immunization of a suitable subject with an immunogenic TANGO 130 preparation induces a polyclonal anti-TANGO 130 antibody response.

Accordingly, another aspect of the invention pertains to anti-TANGO 130 antibodies. The term “antibody” as used herein refers to immunoglobulin molecules and immunologically active portions of immunoglobulin molecules, i.e., molecules that contain an antigen binding site which specifically binds an antigen, such as TANGO 130. A molecule which specifically binds to TANGO 130 is a molecule which binds TANGO 130, but does not substantially bind other molecules in a sample, e.g., a biological sample, which naturally contains TANGO 130. Examples of immunologically active portions of immunoglobulin molecules include F(ab) and F(ab′)₂ fragments which can be generated by treating the antibody with an enzyme such as pepsin or papin, respectively. The invention provides polyclonal and monoclonal antibodies that bind TANGO 130. The term “monoclonal antibody” or “monoclonal antibody composition”, as used herein, refers to a population of antibody molecules that contain only one species of an antigen binding site capable of immunoreacting with a particular epitope of TANGO 130. A monoclonal antibody composition thus typically displays a single binding affinity for a particular TANGO 130 protein with which it immunoreacts.

Polyclonal anti-TANGO 130 antibodies can be prepared as described above by immunizing a suitable subject with a TANGO 130 immunogen. The anti-TANGO 130 antibody titer in the immunized subject can be monitored over time by standard techniques, such as with an enzyme linked immunosorbent assay (ELISA) using immobilized TANGO 130. If desired, the antibody molecules directed against TANGO 130 can be isolated from a mammal (e.g., from the blood) and further purified by well-known techniques, such as protein A chromatography to obtain the IgG fraction. At an appropriate time after immunization, e.g., when the anti-TANGO 130 antibody titers are highest, antibody-producing cells can be obtained from the subject and used to prepare monoclonal antibodies by standard techniques, such as the hybridoma technique originally described by Kohler and Milstein (1975) Nature 256:495, the human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with a TANGO 130 immunogen as described above, and the culture supernatants of the resulting hybridoma cells are screened to identify a hybridoma producing a monoclonal antibody that binds TANGO 130.

Any of the many well known protocols used for fusing lymphocytes and immortalized cell lines can be applied for the purpose of generating an anti-TANGO 130 monoclonal antibody (see, e.g., Current Protocols in Immunology, supra; Galfre et al. (1977) Nature 266:550; R. H. Kenneth, in Monoclonal Antibodies: A New Dimension In Biological Analyses, Plenum Publishing Corp., New York, N.Y. (1980); and Lerner (1981) Yale J. Biol. Med., 54:387. Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line, e.g., a myeloma cell line that is sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind TANGO 130, e.g., using a standard ELISA assay.

Alternative to preparing monoclonal antibody-secreting hybridomas, a monoclonal anti-TANGO 130 antibody can be identified and isolated by screening a recombinant combinatorial immunoglobulin library (e.g., an antibody phage display library) with TANGO 130 to thereby isolate immunoglobulin library members that bind TANGO 130. Kits for generating and screening phage display libraries are commercially available (e.g., the Pharmacia Recombinant Phage Antibody System, Catalog No. 27-9400-01; and the Stratagene SurjZAP (Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) BioTechniques 9:1370; Hay et al. (1992) Hum. Antibod. Hybridomas 3:81; Huse et al. (1989) Science 246:1275; Griffiths et al. (1993) EMBO J. 12:725.

Additionally, recombinant anti-TANGO 130 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041; Liu et al. (1987) Proc. Natl. Acad. Sci. USA 84:3439; Liu et al. (1987) J. Immunol. 139:3521; Sun et al. (1987) Proc. Natl. Acad. Sci. USA 84:214; Nishimura et al. (1987) Canc. Res. 47:999; Wood et al. (1985) Nature 314:446; and Shaw et al. (1988) J. Natl. Cancer Inst. 80:1553); Morrison (1985) Science 229:1202; Oi et al. (1986) BioTechniques 4:214; U.S. Pat. No. 5,225,539; Jones et al. (1986) Nature 321:552; Verhoeyan et al. (1988) Science 239:1534; and Beidler et al. (1988) J. Immunol. 141:4053.

Completely human antibodies are particularly desirable for therapeutic treatment of human patients. Such antibodies can be produced using transgenic mice which are incapable of expressing endogenous immunoglobulin heavy and light chain genes, but which can express human heavy and light chain genes. The transgenic mice are immunized in the normal fashion with a selected antigen, e.g., all or a portion of TANGO 130. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65. For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope.

First, a non-human monoclonal antibody which binds a selected antigen (epitope), e.g., an antibody which inhibits TANGO 130 activity, is identified. The heavy chain and the light chain of the non-human antibody are cloned and used to create phage display Fab fragments. For example, the heavy chain gene can be cloned into a plasmid vector so that the heavy chain can be secreted from bacteria. The light chain gene can be cloned into a phage coat protein gene so that the light chain can be expressed on the surface of phage. A repertoire (random collection) of human light chains fused to phage is used to infect the bacteria which express the non-human heavy chain. The resulting progeny phage display hybrid antibodies (human light chain/non-human heavy chain). The selected antigen is used in a panning screen to select phage which bind the selected antigen. Several rounds of selection may be required to identify such phage. Next, human light chain genes are isolated from the selected phage which bind the selected antigen. These selected human light chain genes are then used to guide the selection of human heavy chain genes as follows. The selected human light chain genes are inserted into vectors for expression by bacteria. Bacteria expressing the selected human light chains are infected with a repertoire of human heavy chains fused to phage. The resulting progeny phage display human antibodies (human light chain/human heavy chain).

Next, the selected antigen is used in a panning screen to select phage which bind the selected antigen. The phage selected in this step display a completely human antibody which recognizes the same epitope recognized by the original selected, non-human monoclonal antibody. The genes encoding both the heavy and light chains are readily isolated and can be further manipulated for production of human antibody. This technology is described by Jespers et al. (1994) Biotechnology 12:899.

An anti-TANGO 130 antibody (e.g., monoclonal antibody) can be used to isolate TANGO 130 by standard techniques, such as affinity chromatography or immunoprecipitation. An anti-TANGO 130 antibody can facilitate the purification of natural TANGO 130 from cells and of recombinantly produced TANGO 130 expressed in host cells. Moreover, an anti-TANGO 130 antibody can be used to detect TANGO 130 protein (e.g., in a cellular lysate or cell supernatant) in order to evaluate the abundance and pattern of expression of the TANGO 130 protein. Anti-TANGO 130 antibodies can be used diagnostically to monitor protein levels in tissue as part of a clinical testing procedure, e.g., to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, (-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

An antibody (or fragment thereof) can be conjugated to a therapeutic moiety such as a cytotoxin, a therapeutic agent, or a radioactive agent (e.g., a radioactive metal ion). Cytotoxins and cytotoxic agents include any agent that is detrimental to cells. Examples of such agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, and 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin {formerly designated daunomycin} and doxorubicin), antibiotics (e.g., dactinomycin {formerly designated actinomycin}, bleomycin, mithramycin, and anthramycin), and anti-mitotic agents (e.g., vincristine and vinblastine).

Conjugated antibodies of the invention can be used for modifying a given biological response, the drug moiety not being limited to classical chemical therapeutic agents. For example, the drug moiety can be a protein or polypeptide possessing a desired biological activity. Such proteins include, for example, toxins such as abrin, ricin A, Pseudomonas exotoxin, or diphtheria toxin; proteins such as tumor necrosis factor, alpha-interferon, beta-interferon, nerve growth factor, platelet derived growth factor, tissue plasminogen activator; and biological response modifiers such as lymphokines, interleukin-1, interleukin-2, interleukin-6, granulocyte macrophage colony stimulating factor, granulocyte colony stimulating factor, or other growth factors.

Techniques for conjugating a therapeutic moiety to an antibody are well known (see, e.g., Amon et al., 1985, “Monoclonal Antibodies For Immunotargeting Of Drugs In Cancer Therapy”, in Monoclonal Antibodies And Cancer Therapy, Reisfeld et al., Eds., Alan R. Liss, Inc. pp. 243-256; Hellstrom et al., 1987, “Antibodies For Drug Delivery”, in Controlled Drug Delivery, 2nd ed., Robinson et al., Eds., Marcel Dekker, Inc., pp. 623-653; Thorpe, 1985, “Antibody Carriers Of Cytotoxic Agents In Cancer Therapy: A Review”, in Monoclonal Antibodies '84: Biological And Clinical Applications, Pinchera et al., Eds., pp. 475-506; “Analysis, Results, And Future Prospective Of The Therapeutic Use Of Radiolabeled Antibody In Cancer Therapy”, in Monoclonal Antibodies For Cancer Detection And Therapy, Baldwin et al., Eds., Academic Press, pp. 303-316, 1985; and Thorpe et al., 1982, Immunol. Rev., 62:119-158). Alternatively, an antibody can be conjugated to a second antibody to form an antibody heteroconjugate as described by Segal in U.S. Pat. No. 4,676,980.

III. Recombinant Expression Vectors and Host Cells

Another aspect of the invention pertains to vectors, preferably expression vectors, containing a nucleic acid encoding TANGO 130 (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., TANGO 130 proteins, mutant forms of TANGO 130, fusion proteins, etc.).

The recombinant expression vectors of the invention can be designed for expression of TANGO 130 in prokaryotic or eukaryotic cells, e.g., bacterial cells such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident (prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111). Such alteration of nucleic acid sequences of the invention can be carried out by standard DNA synthesis techniques.

In another embodiment, the TANGO 130 expression vector is a yeast expression vector. Examples of vectors for expression in yeast S. cerivisae include pYepSec1 (Baldari et al. (1987) EMBO J. 6:229), pMFa (Kurjan and Herskowitz, (1982) Cell 30:933), pJRY88 (Schultz et al. (1987) Gene 54:113), pYES2 (Invitrogen Corporation, San Diego, Calif.), and pPicZ (InVitrogen Corp, San Diego, Calif.).

Alternatively, TANGO 130 can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156) and the pVL series (Lucklow and Summers (1989) Virology 170:31).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268), lymphoid-specific promoters (Calame and Eaton (1988) Adv. Immunol. 43:235), in particular promoters of T cell receptors (Winoto and Baltimore (1989) EMBO J. 8:729) and immunoglobulins (Banerji et al. (1983) Cell 33:729; Queen and Baltimore (1983) Cell 33:741), neuron-specific promoters (e.g., the neurofilament promoter; Byrne and Ruddle (1989) Proc. Natl. Acad. Sci. USA 86:5473), pancreas-specific promoters (Edlund et al. (1985) Science 230:912), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374) and the (-fetoprotein promoter (Campes and Tilghman (1989) Genes Dev. 3:537).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to TANGO 130 mRNA. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, TANGO 130 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding TANGO 130 or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) TANGO 130 protein. Accordingly, the invention further provides methods for producing TANGO 130 protein using the host cells of the invention. In one embodiment, the method comprises culturing the host cell of invention (into which a recombinant expression vector encoding TANGO 130 has been introduced) in a suitable medium such that TANGO 130 protein is produced. In another embodiment, the method further comprises isolating TANGO 130 from the medium or the host cell.

The host cells of the invention can also be used to produce nonhuman transgenic animals. For example, in one embodiment, a host cell of the invention is a fertilized oocyte or an embryonic stem cell into which TANGO 130-coding sequences have been introduced. Such host cells can then be used to create non-human transgenic animals in which exogenous TANGO 130 sequences have been introduced into their genome or homologous recombinant animals in which endogenous TANGO 130 sequences have been altered. Such animals are useful for studying the function and/or activity of TANGO 130 and for identifying and/or evaluating modulators of TANGO 130 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous TANGO 130 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing TANGO 130-encoding nucleic acid into the male pronuclei of a fertilized oocyte, e.g., by microinjection, retroviral infection, and allowing the oocyte to develop in a pseudopregnant female foster animal. The TANGO 130 cDNA sequence e.g., that of (SEQ ID NO:1, SEQ ID NO:7, SEQ ID NO:14, the cDNA of ATCC 98823, the cDNA of ATCC 98844, or the cDNA of ATCC 98845) can be introduced as a transgene into the genome of a non-human animal. Alternatively, a nonhuman homologue of the human TANGO 130 gene, such as the mouse TANGO 130 gene, can be used as a transgene. Intronic sequences and polyadenylation signals can also be included in the transgene to increase the efficiency of expression of the transgene. A tissue-specific regulatory sequence(s) can be operably linked to the TANGO 130 transgene to direct expression of TANGO 130 protein to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the TANGO 130 transgene in its genome and/or expression of TANGO 130 mRNA in tissues or cells of the animals. A transgenic founder animal can then be used to breed additional animals carrying the transgene. Moreover, transgenic animals carrying a transgene encoding TANGO 130 can further be bred to other transgenic animals carrying other transgenes.

To create an homologous recombinant animal, a vector is prepared which contains at least a portion of a TANGO 130 gene (e.g., a human or a non-human homolog of the TANGO 130 gene, e.g., the murine TANGO 130 gene) into which a deletion, addition or substitution has been introduced to thereby alter, e.g., functionally disrupt, the TANGO 130 gene. In a preferred embodiment, the vector is designed such that, upon homologous recombination, the endogenous TANGO 130 gene is functionally disrupted (i.e., no longer encodes a functional protein; also referred to as a “knock out” vector). Alternatively, the vector can be designed such that, upon homologous recombination, the endogenous TANGO 130 gene is mutated or otherwise altered but still encodes functional protein (e.g., the upstream regulatory region can be altered to thereby alter the expression of the endogenous TANGO 130 protein). In the homologous recombination vector, the altered portion of the TANGO 130 gene is flanked at its 5′ and 3′ ends by additional nucleic acid of the TANGO 130 gene to allow for homologous recombination to occur between the exogenous TANGO 130 gene carried by the vector and an endogenous TANGO 130 gene in an embryonic stem cell. The additional flanking TANGO 130 nucleic acid is of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation) and cells in which the introduced TANGO 130 gene has homologously recombined with the endogenous TANGO 130 gene are selected (see, e.g., Li et al. (1992) Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication Nos. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232. Another example of a recombinase system is the FLP recombinase system of Saccharomyces cerevisiae (O'Gorman et al. (1991) Science 251:1351). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810 and PCT Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The TANGO 130 nucleic acid molecules, TANGO 130 proteins, and anti-TANGO 130 antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid of the invention. Such methods comprise formulating a pharmaceutically acceptable carrier with an agent which modulates expression or activity of a polypeptide or nucleic acid of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid of the invention and one or more additional active compounds.

The agent which modulates expression or activity can, for example, be a small molecule. For example, such small molecules include peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents and protein or polypeptide agents depends upon a number of factors within the ken of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid or polypeptide of the invention. Examples of doses of a small molecule include milligram or microgram amounts per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). Examples of doses of a protein or polypeptide include gram, milligram or microgram amounts per kilogram of subject or sample weight (e.g., about 1 microgram per kilogram to about 5 grams per kilogram, about 100 micrograms per kilogram to about 500 milligrams per kilogram, or about 1 milligram per kilogram to about 50 milligrams per kilogram). For antibodies, examples of dosages are from about 0.1 milligram per kilogram to 100 milligrams per kilogram of body weight (generally 10 milligrams per kilogram to 20 milligrams per kilogram). If the antibody is to act in the brain, a dosage of 50 milligrams per kilogram to 100 milligrams per kilogram is usually appropriate. It is furthermore understood that appropriate doses of one of these agents depend upon the potency of the agent with respect to the expression or activity to be modulated. Such appropriate doses can be determined using the assays described herein. When one or more of these agents is to be administered to an animal (e.g., a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetra-acetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL((BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a TANGO 130 protein or anti-TANGO 130 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. Depending on the type and severity of the disease, about 1 μg/kg to 15 mg/kg (e.g., 0.1 to 20 mg/kg) of active compound, e.g., an antibody, is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays. An exemplary dosing regimen is disclosed in WO 94/04188. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see, e.g., Chen et al. (1994) Proc. Natl. Acad. Sci. USA 91:3054). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

It is recognized that the pharmaceutical compositions and methods described herein can be used independently or in combination with one another. That is, subjects can be administered one or more of the pharmaceutical compositions, e.g., pharmaceutical compositions comprising a nucleic acid molecule or protein of the invention or a modulator thereof, subjected to one or more of the therapeutic methods described herein, or both, in temporally overlapping or non-overlapping regimens. When therapies overlap temporally, the therapies may generally occur in any order and can be simultaneous (e.g., administered simultaneously together in a composite composition or simultaneously but as separate compositions) or interspersed. By way of example, a subject afflicted with a disorder described herein can be simultaneously or sequentially administered both a cytotoxic agent which selectively kills aberrant cells and an antibody (e.g., an antibody of the invention) which can, in one embodiment, be conjugated or linked with a therapeutic agent, a cytotoxic agent, an imaging agent, or the like.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: a) screening assays; b) detection assays (e.g., chromosomal mapping, tissue typing, forensic biology); c) predictive medicine (e.g., diagnostic assays, prognostic assays, monitoring clinical trials, and pharmacogenomics); and d) methods of treatment (e.g., therapeutic and prophylactic). A TANGO 130 protein interacts with other cellular proteins and can thus be used for (i) regulation of cellular proliferation; (ii) regulation of cellular differentiation; and (iii) regulation of cell survival. The isolated nucleic acid molecules of the invention can be used to express TANGO 130 protein (e.g., via a recombinant expression vector in a host cell in gene therapy applications), to detect TANGO 130 mRNA (e.g., in a biological sample) or a genetic lesion in a TANGO 130 gene, and to modulate TANGO 130 activity. In addition, the TANGO 130 proteins can be used to screen drugs or compounds which modulate the TANGO 130 activity or expression as well as to treat disorders characterized by insufficient or excessive production of TANGO 130 protein or production of TANGO 130 protein forms which have decreased or aberrant activity compared to TANGO 130 wild type protein. In addition, the anti-TANGO 130 antibodies of the invention can be used to detect and isolate TANGO 130 proteins and modulate TANGO 130 activity.

This invention further pertains to novel agents identified by the above-described screening assays and uses thereof for treatments as described herein.

A. Screening Assays

The invention provides a method (also referred to herein as a “screening assay”) for identifying modulators, i.e., candidate or test compounds or agents (e.g., peptides, peptidomimetics, small molecules or other drugs) which bind to TANGO 130 proteins or have a stimulatory or inhibitory effect on, for example, TANGO 130 expression or TANGO 130 activity.

In one embodiment, the invention provides assays for screening candidate or test compounds which bind to or modulate the activity of a membrane-bound form of a TANGO 130 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) BioTechniques 13:412), or on beads (Lam (1991) Nature 354:82), chips (Fodor (1993) Nature 364:555), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865) or phage (Scott and Smith (1990) Science 249:386; Devlin (1990) Science 249:404; Cwirla et al. (1990) Proc. Natl. Acad. Sci. USA 87:6378; and Felici (1991) J. Mol. Biol. 222:301).

TANGO 130 molecules of the invention may include forms of TANGO 130 which are membrane-bound. Such membrane-bound forms may be naturally occuring forms of TANGO 130, or they may be forms that have been modified such that they are expressed as membrane-bound proteins, e.g., by expressing TANGO 130 molecules of the invention which have been operably linked to a heterologous transmembrane sequence at their carboxy terminus. Accordingly, in one embodiment, an assay is a cell-based assay in which a cell which expresses a membrane-bound form of TANGO 130 protein, or a biologically active portion thereof, on the cell surface is contacted with a test compound and the ability of the test compound to bind to a TANGO 130 protein determined. The cell, for example, can be a yeast cell or a cell of mammalian origin. Determining the ability of the test compound to bind to the TANGO 130 protein can be accomplished, for example, by coupling the test compound with a radioisotope or enzymatic label such that binding of the test compound to the TANGO 130 protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In a preferred embodiment, the assay comprises contacting a cell which expresses a membrane-bound form of TANGO 130 protein, or a biologically active portion thereof, on the cell surface with a known compound which binds TANGO 130 to form an assay mixture, contacting the assay mixture with a test compound, and determining the ability of the test compound to interact with a TANGO 130 protein, wherein determining the ability of the test compound to interact with a TANGO 130 protein comprises determining the ability of the test compound to preferentially bind to TANGO 130 or a biologically active portion thereof as compared to the known compound.

In another embodiment, an assay is a cell-based assay comprising contacting a cell expressing a membrane-bound form of TANGO 130 protein, or a biologically active portion thereof, on the cell surface with a test compound and determining the ability of the test compound to modulate (e.g., stimulate or inhibit) the activity of the TANGO 130 protein or biologically active portion thereof. Determining the ability of the test compound to modulate the activity of TANGO 130 or a biologically active portion thereof can be accomplished, for example, by determining the ability of the TANGO 130 protein to bind to or interact with a TANGO 130 target molecule.

As used herein, a “target molecule” is a molecule with which a TANGO 130 protein binds or interacts in nature, for example, a molecule on the surface of a cell which expresses a TANGO 130 protein, a molecule on the surface of a second cell, a molecule in the extracellular milieu, a molecule associated with the internal surface of a cell membrane or a cytoplasmic molecule. A TANGO 130 target molecule can be a non-TANGO 130 molecule or a TANGO 130 protein or polypeptide of the present invention. In one embodiment, a TANGO 130 target molecule is a component of a signal transduction pathway which facilitates transduction of an extracellular signal (e.g., a signal generated by binding of a compound to a membrane-bound TANGO 130 molecule) through the cell membrane and into the cell. The target, for example, can be a second intercellular protein which has catalytic activity or a protein which facilitates the association of downstream signaling molecules with TANGO 130.

Determining the ability of the TANGO 130 protein to bind to or interact with a TANGO 130 target molecule can be accomplished by one of the methods described above for determining direct binding. In a preferred embodiment, determining the ability of the TANGO 130 protein to bind to or interact with a TANGO 130 target molecule can be accomplished by determining the activity of the target molecule. For example, the activity of the target molecule can be determined by detecting induction of a cellular second messenger of the target (e.g., intracellular Ca²⁺, diacylglycerol, IP₃, etc.), detecting catalytic/enzymatic activity of the target on an appropriate substrate, detecting the induction of a reporter gene (e.g., a TANGO 130-responsive regulatory element operably linked to a nucleic acid encoding a detectable marker, e.g., luciferase), or detecting a cellular response, for example, cellular differentiation, or cell proliferation.

In yet another embodiment, an assay of the present invention is a cell-free assay comprising contacting a TANGO 130 protein or biologically active portion thereof with a test compound and determining the ability of the test compound to bind to the TANGO 130 protein or biologically active portion thereof. Binding of the test compound to the TANGO 130 protein can be determined either directly or indirectly as described above. In a preferred embodiment, the assay includes contacting the TANGO 130 protein or biologically active portion thereof with a known compound which binds TANGO 130 to form an assay mixture, contacting the assay mixture with a test compound, and determining the ability of the test compound to interact with a TANGO 130 protein, wherein determining the ability of the test compound to interact with a TANGO 130 protein comprises determining the ability of the test compound to preferentially bind to TANGO 130 or biologically active portion thereof as compared to the known compound.

In another embodiment, an assay is a cell-free assay comprising contacting TANGO 130 protein or biologically active portion thereof with a test compound and determining the ability of the test compound to modulate (e.g., stimulate or inhibit) the activity of the TANGO 130 protein or biologically active portion thereof. Determining the ability of the test compound to modulate the activity of TANGO 130 can be accomplished, for example, by determining the ability of the TANGO 130 protein to bind to a TANGO 130 target molecule by one of the methods described above for determining direct binding. In an alternative embodiment, determining the ability of the test compound to modulate the activity of TANGO 130 can be accomplished by determining the ability of the TANGO 130 protein to further modulate a TANGO 130 target molecule. For example, the catalytic/enzymatic activity of the target molecule on an appropriate substrate can be determined as previously described.

In yet another embodiment, the cell-free assay comprises contacting the TANGO 130 protein or biologically active portion thereof with a known compound which binds TANGO 130 to form an assay mixture, contacting the assay mixture with a test compound, and determining the ability of the test compound to interact with a TANGO 130 protein, wherein determining the ability of the test compound to interact with a TANGO 130 protein comprises determining the ability of the TANGO 130 protein to preferentially bind to or modulate the activity of a TANGO 130 target molecule.

The cell-free assays of the present invention are amenable to use of both soluble or membrane-bound forms of TANGO 130. In the case of cell-free assays comprising a membrane-bound form of TANGO 130, it may be desirable to utilize a solubilizing agent such that the membrane-bound form of TANGO 130 is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton X-100, Triton X-1 14, Thesit™, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either TANGO 130 or its target molecule to facilitate separation of complexed from uncomplexed forms of one or both of the proteins, as well as to accommodate automation of the assay. Binding of a test compound to TANGO 130, or interaction of TANGO 130 with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/TANGO 130 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or TANGO 130 protein, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of TANGO 130 binding or activity determined using standard techniques.

Other techniques for immobilizing proteins on matrices can also be used in the screening assays of the invention. For example, either TANGO 130 or its target molecule can be immobilized utilizing conjugation of biotin and streptavidin. Biotinylated TANGO 130 or target molecules can be prepared from biotin-NHS (N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with TANGO 130 or target molecules but which do not interfere with binding of the TANGO 130 protein to its target molecule can be derivatized to the wells of the plate, and unbound target or TANGO 130 trapped in the wells by antibody conjugation. Methods for detecting such complexes, in addition to those described above for the GST-immobilized complexes, include immunodetection of complexes using antibodies reactive with the TANGO 130 or target molecule, as well as enzyme-linked assays which rely on detecting an enzymatic activity associated with the TANGO 130 or target molecule.

In another embodiment, modulators of TANGO 130 expression are identified in a method in which a cell is contacted with a candidate compound and the expression of TANGO 130 mRNA or protein in the cell is determined. The level of expression of TANGO 130 mRNA or protein in the presence of the candidate compound is compared to the level of expression of TANGO 130 mRNA or protein in the absence of the candidate compound. The candidate compound can then be identified as a modulator of TANGO 130 expression based on this comparison. For example, when expression of TANGO 130 mRNA or protein is greater (statistically significantly greater) in the presence of the candidate compound than in its absence, the candidate compound is identified as a stimulator of TANGO 130 mRNA or protein expression. Alternatively, when expression of TANGO 130 mRNA or protein is less (statistically significantly less) in the presence of the candidate compound than in its absence, the candidate compound is identified as an inhibitor of TANGO 130 mRNA or protein expression. The level of TANGO 130 mRNA or protein expression in the cells can be determined by methods described herein for detecting TANGO 130 mRNA or protein.

In yet another aspect of the invention, the TANGO 130 proteins can be used as “bait proteins” in a two-hybrid assay or three hybrid assay (see, e.g., U.S. Pat. No. 5,283,317; Zervos et al. (1993) Cell 72:223; Madura et al. (1993) J. Biol. Chem. 268:12046; Bartel et al. (1993) BioTechniques 14:920; Iwabuchi et al. (1993) Oncogene 8:1693; and PCT Publication No. WO 94/10300), to identify other proteins, which bind to or interact with TANGO 130 (“TANGO 130-binding proteins” or “TANGO 130-bp”) and modulate TANGO 130 activity. Such TANGO 130-binding proteins are also likely to be involved in the propagation of signals by the TANGO 130 proteins as, for example, upstream or downstream elements of the TANGO 130 pathway.

This invention further pertains to novel agents identified by the above-described screening assays and uses thereof for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. Accordingly, TANGO 130 nucleic acid molecules described herein or fragments thereof, can be used to map the location of TANGO 130 genes on a chromosome. The mapping of the TANGO 130 sequences to chromosomes is an important first step in correlating these sequences with genes associated with disease. For example, TANGO 130 was mapped to chromosome 5 between markers D5Mit195 and D5Mit15, a region syntenic to human 7q, 7p, 18p1, 4p1, 14q.

Briefly, TANGO 130 genes can be mapped to chromosomes by preparing PCR primers (preferably 15-25 bp in length) from the TANGO 130 sequences. Computer analysis of TANGO 130 sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the TANGO 130 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow (because they lack a particular enzyme), but in which human cells can grow, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio et al. (1983) Science 220:919). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the TANGO 130 sequences to design oligonucleotide primers, sublocalization can be achieved with panels of fragments from specific chromosomes. Other mapping strategies which can similarly be used to map a TANGO 130 sequence to its chromosome include in situ hybridization (described in Fan et al. (1990) Proc. Natl. Acad. Sci. USA 87:6223), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical, e.g., colcemid, that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., (Human Chromosomes: A Manual of Basic Techniques (Pergamon Press, New York, 1988)).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al. (1987) Nature 325:783.

Moreover, differences in the DNA sequences between individuals affected and unaffected with a disease associated with the TANGO 130 gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The TANGO 130 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the TANGO 130 sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The TANGO 130 sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NOs:1, 7, and 14 can comfortably provide positive individual identification with a panel of perhaps 10 to 1,000 primers which each yield a noncoding amplified sequence of 100 bases. If predicted coding sequences, such as those in SEQ ID NOs:2, 8, and 15 are used, a more appropriate number of primers for positive individual identification would be 500-2,000.

If a panel of reagents from TANGO 130 sequences described herein is used to generate a unique identification database for an individual, those same reagents can later be used to identify tissue from that individual. Using the unique identification database, positive identification of the individual, living or dead, can be made from extremely small tissue samples.

3. Use of Partial TANGO 130 Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:7 are particularly appropriate for this use as greater numbers of polymorphisms occur in the noncoding regions, making it easier to differentiate individuals using this technique. Examples of polynucleotide reagents include the TANGO 130 sequences or portions thereof, e.g., fragments derived from the noncoding regions of SEQ ID NO:7 having a length of at least 20 or 30 bases.

The TANGO 130 sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such TANGO 130 probes can be used to identify tissue by species and/or by organ type.

In a similar fashion, these reagents, e.g., TANGO 130 primers or probes can be used to screen tissue culture for contamination (i.e., screen for the presence of a mixture of different types of cells in a culture).

C. Predictive Medicine

The present invention also pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic assays for determining TANGO 130 protein and/or nucleic acid expression as well as TANGO 130 activity, in the context of a biological sample (e.g., blood, serum, cells, tissue) to thereby determine whether an individual is afflicted with a disease or disorder, or is at risk of developing a disorder, associated with aberrant TANGO 130 expression or activity. The invention also provides for prognostic (or predictive) assays for determining whether an individual is at risk of developing a disorder associated with TANGO 130 protein, nucleic acid expression or activity. For example, mutations in a TANGO 130 gene can be assayed in a biological sample. Such assays can be used for prognostic or predictive purpose to thereby prophylactically treat an individual prior to the onset of a disorder characterized by or associated with TANGO 130 protein, nucleic acid expression or activity.

As an alternative to making determinations based on the absolute expression level of selected genes, determinations may be based on the normalized expression levels of these genes. Expression levels are normalized by correcting the absolute expression level of a a gene encoding a polypeptide of the invention by comparing its expression to the expression of a different gene, e.g., a housekeeping gene that is constitutively expressed. Suitable genes for normalization include housekeeping genes such as the actin gene. This normalization allows the comparison of the expression level in one sample (e.g., a patient sample), to another sample, or between samples from different sources.

Alternatively, the expression level can be provided as a relative expression level. To determine a relative expression level of a gene, the level of expression of the gene is determined for 10 or more samples of different endothelial (e.g. intestinal endothelium, airway endothelium, or other mucosal epithelium) cell isolates, preferably 50 or more samples, prior to the determination of the expression level for the sample in question. The mean expression level of each of the genes assayed in the larger number of samples is determined and this is used as a baseline expression level for the gene(s) in question. The expression level of the gene determined for the test sample (absolute level of expression) is then divided by the mean expression value obtained for that gene. This provides a relative expression level and aids in identifying extreme cases of disorders associated with aberrant expression of a gene encoding a polypeptide of the invention protein or with aberrant expression of a ligand thereof.

Preferably, the samples used in the baseline determination will be from either or both of cells which aberrantly express a gene encoding a polypeptide of the invention or a ligand thereof (i.e. ‘diseased cells’) and cells which express a gene encoding a polypeptide of the invention at a normal levelor a ligand thereof (i.e. ‘normal’ cells). The choice of the cell source is dependent on the use of the relative expression level. Using expression found in normal tissues as a mean expression score aids in validating whether aberrance in expression of a gene encoding a polypeptide of the invention occurs specifically in diseased cells. Such a use is particularly important in identifying whether a gene encoding a polypeptide of the invention can serve as a target gene. In addition, as more data is accumulated, the mean expression value can be revised, providing improved relative expression values based on accumulated data. Expression data from endothelial cells (e.g. mucosal endothelial cells) provides a means for grading the severity of the disorder.

Another aspect of the invention provides methods for determining TANGO 130 protein, nucleic acid expression or TANGO 130 activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.) Yet another aspect of the invention pertains to monitoring the influence of agents (e.g., drugs or other compounds) on the expression or activity of TANGO 130 in clinical trials.

These and other agents are described in further detail in the following sections.

1. Diagnostic Assays

An exemplary method for detecting the presence or absence of TANGO 130 in a biological sample involves obtaining a biological sample from a test subject and contacting the biological sample with a compound or an agent capable of detecting TANGO 130 protein or nucleic acid (e.g., mRNA, genomic DNA) that encodes TANGO 130 protein such that the presence of TANGO 130 is detected in the biological sample. A preferred agent for detecting TANGO 130 mRNA or genomic DNA is a labeled nucleic acid probe capable of hybridizing to TANGO 130 mRNA or genomic DNA. The nucleic acid probe can be, for example, a full-length TANGO 130 nucleic acid, such as the nucleic acid of SEQ ID NOs:1, 7, 14, or a portion thereof, such as an oligonucleotide of at least 15, 30, 50, 100, 250 or 500 nucleotides in length and sufficient to specifically hybridize under stringent conditions to TANGO 130 mRNA or genomic DNA. Other suitable probes for use in the diagnostic assays of the invention are described herein.

A preferred agent for detecting TANGO 130 protein is an antibody capable of binding to TANGO 130 protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling,of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect TANGO 130 mRNA, protein, or genomic DNA in a biological sample in vitro as well as in vivo. For example, in vitro techniques for detection of TANGO 130 mRNA include Northern hybridizations and in situ hybridizations. In vitro techniques for detection of TANGO 130 protein include enzyme linked immunosorbent assays (ELISAs), Western blots, immunoprecipitations and immunofluorescence. In vitro techniques for detection of TANGO 130 genomic DNA include Southern hybridizations. Furthermore, in vivo techniques for detection of TANGO 130 protein include introducing into a subject a labeled anti-TANGO 130 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting TANGO 130 protein, mRNA, or genomic DNA, such that the presence of TANGO 130 protein, mRNA or genomic DNA is detected in the biological sample, and comparing the presence of TANGO 130 protein, mRNA or genomic DNA in the control sample with the presence of TANGO 130 protein, mRNA or genomic DNA in the test sample.

The invention also encompasses kits for detecting the presence of TANGO 130 in a biological sample (a test sample). Such kits can be used to determine if a subject is suffering from or is at increased risk of developing a disorder associated with aberrant expression of TANGO 130 (e.g., a cell differentiation or cell proliferation disorder). For example, the kit can comprise a labeled compound or agent capable of detecting TANGO 130 protein or mRNA in a biological sample and means for determining the amount of TANGO 130 in the sample (e.g., an anti-TANGO 130 antibody or an oligonucleotide probe which binds to DNA encoding TANGO 130, e.g., to SEQ ID NO:7). Kits can also include instructions for observing that the tested subject is suffering from or is at risk of developing a disorder associated with aberrant expression of TANGO 130 if the amount of TANGO 130 protein or mRNA is above or below a normal level.

For antibody-based kits, the kit can comprise, for example: (1) a first antibody (e.g., attached to a solid support) which binds to TANGO 130 protein; and, optionally, (2) a second, different antibody which binds to TANGO 130 protein or the first antibody and is conjugated to a detectable agent.

For oligonucleotide-based kits, the kit can comprise, for example: (1) an oligonucleotide, e.g., a detectably labelled oligonucleotide, which hybridizes to a TANGO 130 nucleic acid sequence or (2) a pair of primers useful for amplifying a TANGO 130 nucleic acid molecule.

The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples which can be assayed and compared to the test sample contained. Each component of the kit is usually enclosed within an individual container and all of the various containers are within a single package along with instructions for observing whether the tested subject is suffering from or is at risk of developing a disorder associated with aberrant expression of TANGO 130.

2. Prognostic Assays

The methods described herein can furthermore be utilized as diagnostic or prognostic assays to identify subjects having or at risk of developing a disease or disorder associated with aberrant TANGO 130 expression or activity. For example, the assays described herein, such as the preceding diagnostic assays or the following assays, can be utilized to identify a subject having or at risk of developing a disorder associated with TANGO 130 protein and/or nucleic acid expression or activity, e.g., a disorder characterized by aberrant TANGO 130 protein or nucleic acid expression marked by abnormal cellular growth or by abnormal development. Alternatively, the prognostic assays can be utilized to identify a subject having or at risk for developing such a disease or disorder. Thus, the present invention provides a method in which a test sample is obtained from a subject and TANGO 130 protein or nucleic acid (e.g., mRNA, genomic DNA) is detected, wherein the presence of TANGO 130 protein or nucleic acid is diagnostic for a subject having or at risk of developing a disease or disorder associated with aberrant TANGO 130 expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant TANGO 130 expression or activity. For example, such methods can be used to determine whether a subject can be effectively treated with a specific agent or class of agents (e.g., agents of a type which decrease TANGO 130 activity). Thus, the present invention provides methods for determining whether a subject can be effectively treated with an agent for a disorder associated with aberrant TANGO 130 expression or activity in which a test sample is obtained and TANGO 130 protein or nucleic acid is detected (e.g., wherein the presence of TANGO 130 protein or nucleic acid is diagnostic for a subject that can be administered the agent to treat a disorder associated with aberrant TANGO 130 expression or activity).

The methods of the invention can also be used to detect genetic lesions or mutations in a TANGO 130 gene, thereby determining if a subject with the lesioned gene is at risk for a disorder characterized by aberrant cell proliferation and/or differentiation. In preferred embodiments, the methods include detecting, in a sample of cells from the subject, the presence or absence of a genetic lesion or mutation characterized by at least one of an alteration affecting the integrity of a gene encoding a TANGO 130-protein, or the mis-expression of the TANGO 130 gene. For example, such genetic lesions or mutations can be detected by ascertaining the existence of at least one of: 1) a deletion of one or more nucleotides from a TANGO 130 gene; 2) an addition of one or more nucleotides to a TANGO 130 gene; 3) a substitution of one or more nucleotides of a TANGO 130 gene; 4) a chromosomal rearrangement of a TANGO 130 gene; 5) an alteration in the level of a messenger RNA transcript of a TANGO 130 gene; 6) an aberrant modification of a TANGO 130 gene, such as of the methylation pattern of the genomic DNA; 7) the presence of a non-wild type splicing pattern of a messenger RNA transcript of a TANGO 130 gene; 8) a non-wild type level of a TANGO 130-protein; 9) an allelic loss of a TANGO 130 gene; and 10) an inappropriate post-translational modification of a TANGO 130-protein. As described herein, there are a large number of assay techniques known in the art which can be used for detecting lesions in a TANGO 130 gene. A preferred biological sample is a peripheral blood leukocyte sample isolated by conventional means from a subject.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077; and Nakazawa et al. (1994) Proc. Natl. Acad. Sci. USA 91:360), the latter of which can be particularly useful for detecting point mutations in the TANGO 130 gene (see, e.g., Abravaya et al. (1995) Nucleic Acids Res. 23:675). This method can include the steps of collecting a sample of cells from a patient, isolating nucleic acid (e.g., genomic, mRNA or both) from the cells of the sample, contacting the nucleic acid sample with one or more primers which specifically hybridize to a TANGO 130 gene under conditions such that hybridization and amplification of the TANGO 130-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874), transcriptional amplification system (Kwoh, et al. (1989) Proc. Natl. Acad. Sci. USA 86:1173), Q-Beta Replicase (Lizardi et al. (1988) BioTechniques 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a TANGO 130 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations in TANGO 130 can be identified by hybridizing a sample and control nucleic acids, e.g., DNA or RNA, to high density arrays containing hundreds or thousands of oligonucleotides probes (Cronin et al. (1996) Human Mutation 7:244; Kozal et al. (1996) Nature Medicine 2:753). For example, genetic mutations in TANGO 130 can be identified in two-dimensional arrays containing light-generated DNA probes as described in Cronin et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the TANGO 130 gene and detect mutations by comparing the sequence of the sample TANGO 130 with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) BioTechniques 19:448), including sequencing by mass spectrometry (see, e.g., PCT Publication No. WO 94/16101; Cohen et al. (1996) Adv. Chromatogr. 36:127; and Griffin et al. (1993) Appl. Biochem. Biotechnol. 38:147).

Other methods for detecting mutations in the TANGO 130 gene include methods in which protection from cleavage agents is used to detect mismatched bases in RNA/RNA or RNA/DNA heteroduplexes (Myers et al. (1985) Science 230:1242). In general, the technique of “mismatch cleavage” entails providing heteroduplexes formed by hybridizing (labeled) RNA or DNA containing the wild-type TANGO 130 sequence with potentially mutant RNA or DNA obtained from a tissue sample. The double-stranded duplexes are treated with an agent which cleaves single-stranded regions of the duplex such as which will exist due to basepair mismatches between the control and sample strands. RNA/DNA duplexes can be treated with RNase to digest mismatched regions, and DNA/DNA hybrids can be treated with S 1 nuclease to digest mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton et al. (1988) Proc. Natl. Acad. Sci. USA 85:4397; Saleeba et al. (1992) Methods Enzymol. 217:286. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in TANGO 130 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657). According to an exemplary embodiment, a probe based on a TANGO 130 sequence, e.g., a wild-type TANGO 130 sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in TANGO 130 genes. For example, single strand conformation polymorphism (SSCP) may be used to detect differences in electrophoretic mobility between mutant and wild type nucleic acids (Orita et al. (1989) Proc. Natl. Acad. Sci. USA 86:2766; see also Cotton (1993) Mutat. Res. 285:125; Hayashi (1992) Genet. Anal. Tech. Appl. 9:73). Single-stranded DNA fragments of sample and control TANGO 130 nucleic acids will be denatured and allowed to renature. The secondary structure of single-stranded nucleic acids varies according to sequence, and the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437) or at the extreme 3′ end of one primer where, under appropriate conditions, mismatch can prevent or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a TANGO 130 gene.

Furthermore, any cell type or tissue, preferably peripheral blood leukocytes, in which TANGO 130 is expressed may be utilized in the prognostic assays described herein.

3. Pharmacogenomics

Agents, or modulators which have a stimulatory or inhibitory effect on TANGO 130 activity (e.g., TANGO 130 gene expression) as identified by a screening assay described herein can be administered to individuals to treat (prophylactically or therapeutically) disorders (e.g., cell proliferation or cell differentiation disorders) associated with aberrant TANGO 130 activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of TANGO 130 protein, expression of TANGO 130 nucleic acid, or mutation content of TANGO 130 genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of TANGO 130 protein, expression of TANGO 130 nucleic acid, or mutation content of TANGO 130 genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a TANGO 130 modulator, such as a modulator identified by one of the exemplary screening assays described herein.

4. Monitoring of Effects During Clinical Trials

Monitoring the influence of agents (e.g., drugs, compounds) on the expression or activity of TANGO 130 (e.g., the ability to modulate aberrant cell proliferation and/or differentiation) can be applied not only in basic drug screening, but also in clinical trials. For example, the effectiveness of an agent, as determined by a screening assay as described herein, to increase TANGO 130 gene expression, protein levels or protein activity, can be monitored in clinical trials of subjects exhibiting decreased TANGO 130 gene expression, protein levels, or protein activity. Alternatively, the effectiveness of an agent, as determined by a screening assay, to decrease TANGO 130 gene expression, protein levels or protein activity, can be monitored in clinical trials of subjects exhibiting increased TANGO 130 gene expression, protein levels, or protein activity. In such clinical trials, TANGO 130 expression or activity and preferably, that of other genes that have been implicated in for example, a cellular proliferation disorder, can be used as a marker of the immune responsiveness of a particular cell.

For example, and not by way of limitation, genes, including TANGO 130, that are modulated in cells by treatment with an agent (e.g., compound, drug or small molecule) which modulates TANGO 130 activity (e.g., as identified in a screening assay described herein) can be identified. Thus, to study the effect of agents on cellular proliferation disorders, for example, in a clinical trial, cells can be isolated and RNA prepared and analyzed for the levels of expression of TANGO 130 and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of TANGO 130 or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of a TANGO 130 protein, mRNA, or genomic DNA in the preadministration sample; (iii) obtaining one or more post-administration samples from the subject; (iv) detecting the level of expression or activity of the TANGO 130 protein, mRNA, or genomic DNA in the post-administration samples; (v) comparing the level of expression or activity of the TANGO 130 protein, mRNA, or genomic DNA in the pre-administration sample with the TANGO 130 protein, mRNA, or genomic DNA in the post administration sample or samples; and (vi) altering the administration of the agent to the subject accordingly. For example, increased administration of the agent may be desirable to increase the expression or activity of TANGO 130 to higher levels than detected, i.e., to increase the effectiveness of the agent. Alternatively, decreased administration of the agent may be desirable to decrease expression or activity of TANGO 130 to lower levels than detected, i.e., to decrease the effectiveness of the agent.

C. Methods of Treatment

The present invention provides for both prophylactic and therapeutic methods of treating a subject at risk of (or susceptible to) a disorder or having a disorder associated with aberrant TANGO 130 expression or activity. Such disorders include developmental disorders and growth disorders, e.g., cancer, and others are described elsewhere in this disclosure.

1. Prophylactic Methods

In one aspect, the invention provides a method for preventing in a subject, a disease or condition associated with an aberrant TANGO 130 expression or activity, by administering to the subject an agent which modulates TANGO 130 expression or at least one TANGO 130 activity. Subjects at risk for a disease which is caused or contributed to by aberrant TANGO 130 expression or activity can be identified by, for example, any or a combination of diagnostic or prognostic assays as described herein. Administration of a prophylactic agent can occur prior to the manifestation of symptoms characteristic of the TANGO 130 aberrancy, such that a disease or disorder is prevented or, alternatively, delayed in its progression. Depending on the type of TANGO 130 aberrancy, for example, a TANGO 130 agonist or TANGO 130 antagonist agent can be used for treating the subject. The appropriate agent can be determined based on screening assays described herein.

2. Therapeutic Methods

Another aspect of the invention pertains to methods of modulating TANGO 130 expression or activity for therapeutic purposes. The modulatory method of the invention involves contacting a cell with an agent that modulates one or more of the activities of TANGO 130 protein activity associated with the cell. An agent that modulates TANGO 130 protein activity can be an agent as described herein, such as a nucleic acid or a protein, a naturally-occurring cognate ligand of a TANGO 130 protein, a peptide, a TANGO 130 peptidomimetic, or other small molecule. In one embodiment, the agent stimulates one or more of the biological activities of TANGO 130 protein. Examples of such stimulatory agents include active TANGO 130 protein and a nucleic acid molecule encoding TANGO 130 that has been introduced into the cell. In another embodiment, the agent inhibits one or more of the biological activities of TANGO 130 protein. Examples of such inhibitory agents include antisense TANGO 130 nucleic acid molecules and anti-TANGO 130 antibodies. These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by aberrant expression or activity of a TANGO 130 protein or nucleic acid molecule. In one embodiment, the method involves administering an agent (e.g., an agent identified by a screening assay described herein), or combination of agents that modulates (e.g., upregulates or downregulates) TANGO 130 expression or activity. In another embodiment, the method involves administering a TANGO 130 protein or nucleic acid molecule as therapy to compensate for reduced or aberrant TANGO 130 expression or activity.

Stimulation of TANGO 130 activity is desirable in situations in which TANGO 130 is abnormally downregulated and/or in which increased TANGO 130 activity is likely to have a beneficial effect. Conversely, inhibition of TANGO 130 activity is desirable in situations in which TANGO 130 is abnormally upregulated and/or in which decreased TANGO 130 activity is likely to have a beneficial effect.

This invention is further illustrated by the following examples which should not be construed as limiting. The contents of all references, patents and published patent applications cited throughout this application are hereby incorporated by reference.

### EXAMPLES

### Example 1

### Identification of the TANGO 130 Gene

Murine TANGO 130 was identified in a murine hypothalamus cDNA library. This murine TANGO 130 gene was used to identify a human TANGO 130 gene. The identification and sequencing of both genes is described in this example.

Hypothalamus mRNA Isolation: The murine mRNA used to create the murine hypothalamus library was prepared as follows. Total RNA was isolated from mouse hypothalamus tissue using the guanidinium isothiocyanate/CsCl method of Chirgwin et al. Biochemistry (1979) 18:5294 as described in Current Protocols in Molecular Biology, supra. The RNA was quantitated, diluted to 1 mg/ml in water, and then incubated for 30 minutes at 37° C. with an equal volume of DNase solution (20 mM MgCl₂, 2 mM DTT, 0.1 units DNase, 0.6 units RNase inhibitor in TE) to remove contaminating DNA. The RNA was then extracted with phenol-chloroform-isoamyl alcohol, and ethanol precipitated. After quantitation at 260 nm, an aliquot was electrophoresed to check the integrity of the RNA. Next, PolyA⁺ RNA was isolated using an Oligotex-dT kit from Qiagen (Chatsworth, Calif.) as described by the manufacturer. After quantitation, the mRNA was precipitated in ethanol and resuspended at a concentration of 1 mg/ml in water.

cDNA Library Construction: The isolated hypothalamus mRNA described above was used to prepare cDNA as follows. Hypothalamus mRNA was used as a template for preparation of cDNA according to the method of Gubler et al. (1983) Gene 25:263 using a Superscript Plasmid cDNA synthesis kit (Gibco BRL; Gaithersburg, Md.). The cDNA obtained was ligated into the NotI/SalI sites of the mammalian expression vector pMET7, a modified version of pME18S, which utilizes the SRa promoter as described previously (Takebe (1988) Mol. Cell. Bio. 8:466). Ligated cDNA was transformed into electrocompetent DH10B E. coli either prepared by standard procedures or obtained from Gibco BRL.

DNA Preparation and Sequence Analysis: A number of cDNA clones in the murine hypothalamus library were sequenced to identify sequences of interest. The identified sequences were then used to clone and sequence a complete murine TANGO 130 gene. The identification and analysis were performed as follows.

First, 96-well plates were inoculated with individual hypothalamus library transformants in 1 ml of LB-amp. These inoculations were based on the titers of the cDNA transformants. The resulting cultures were grown for 15 to 16 hours at 37° C. with aeration. Prior to DNA preparation, 100 ml of cell suspension was removed and added to 100 ml of 50% glycerol, mixed and stored at −80° C. (glycerol freeze plate). DNA was then prepared using the Wizard™ miniprep system (Promega, Madison, Wis.) employing modifications for a 96-well format.

The insert cDNAs of a number of clones were sequenced by standard, automated fluorescent dideoxynucleotide sequencing using dye-primer chemistry (Applied Biosystems, Inc., Foster City, Calif.) on Applied Biosystems 373 and 377 sequenators (Applied Biosystems, Foster City, Calif.). The primer used in this sequencing was proximal to the SRa promoter of the vector and therefore selective for the 5′ end of the clones, although other primers with this selectivity can also be used. The short cDNA sequences obtained in this manner were screened as follows.

First, each sequence was checked to determine if it was a bacterial, ribosomal, or mitochondrial contaminant. Such sequences were excluded from the subsequent analysis. Second, sequence artifacts, such as vector and repetitive elements, were masked and/or removed from each sequence. Third, the remaining sequences were searched against a copy of the GenBank™ nucleotide database using the BLASTN program (BLASTN 1.3MP: Altschul et al. (1990) J. Mol. Bio. 215:403). Fourth, the sequences were analyzed against a non-redundant protein database with the BLASTX program (BLASTX 1.3MP: Altschul et al., supra). This protein database is a combination of the Swiss-Prot, PIR, and NCBI GenPept protein databases. The BLASTX program was run using the default BLOSLUM-62 substitution matrix with the filter parameter: “xnu+seg”. The score cutoff utilized was 75.

Assembly of overlapping clones into contigs was done using the program Sequencher (Gene Codes Corp., Ann Arbor, Mich.). The assembled contigs were analyzed using the programs in the GCG package (Genetic Computer Group, Madison, Wis.).

The above-described analysis resulted in the identification of a clone having an open reading frame of 714 amino acids (FIGS. 1A-1D). The protein encoded by this clone was named TANGO 130. The first approximately 24 amino acids in this open reading frame were predicted to be a signal sequence using the method of Von Heijne (1990) J. Membrane Biol. 115:195. The amino-terminal portion of murine TANGO 130 has significant homology to MIA/CD-RAP. This portion is 37% identical to mouse MIA based on a primary sequence alignment of residues 1 to 125 of murine TANGO 130 with murine MIA. The same portion of murine TANGO 130 shows 36% identity to human MIA.

A human heart cDNA library was probed using standard techniques (Sambrook et al. supra) using a ³²P-labeled DNA fragment encoding the full-length mouse TANGO 130. This resulted in the identification of a human (FIGS. 3A-3B) containing an approximately 1.2 kb insert and showing a high degree of homology to the mouse TANGO 130. This clone, named partial human TANGO 130, encodes a polypeptide containing an N-terminal MIA homology domain, with approximately 37% identity to mouse MIA and 38% identity to human MIA. Partial human TANGO 130, however, does not appear to encode the full length human TANGO 130, but lacks coding sequence for approximately 300 amino acids at the carboxy terminus.

Other human clones were pulled from human libraries derived from such tissues as prostate, placenta, and brain. One such clone overlapped the 3′-most region of the partial TANGO 130 cDNA, and extended to the end of the full length TANGO 130 sequence, i.e., the 3′ end of the entire TANGO 130 cDNA. A consensus sequence based on the overlapping regions of sequence, including with partial human TANGO 130 (SEQ ID NO:7), comprises the full length human TANGO 130 sequence (SEQ ID NO:14).

### Example 2

### Distribution of TANGO 130 mRNA in Mouse and Human Tissues

Northern analysis was used to examine TANGO 130 expression in mouse tissues as follows. Northern blots (Mouse Multiple Tissue Northern Blot, Cat.# 7762-1 and Mouse Embryo Multiple Tissue Northern Blot, Cat.# 7763-1; Clontech, Palo Alto, Calif.) containing 2 μg of polyA+ RNA per lane were probed using standard techniques (Chirgwin et al. (1979) Biochemistry 18:5294) with a ³²P-labeled DNA fragment encoding the full-length TANGO 130.

This Northern analysis revealed that an approximately 7 kb TANGO 130 mRNA is expressed in liver, heart, testis, skeletal muscle and brain. This same Northern analysis also revealed the presence of a 1 kb message in testis. These messages are likely to represent alternatively spliced forms of TANGO 130 or the transcription products of related genes. Additionally, an approximately 7 kb message was detected in day 7, day 11, day 15 and day 17 mouse embryo, with highest expression appearing at day 7. Little or no expression was seen in adult spleen, lung, or kidney.

Northern analysis was used to examine TANGO 130 expression in human tissues as follows. Northern blots (Human Multiple Tissue Northern Blots, catalog numbers 7760-1 and 7766-1, Clontech, Palo Alto, Calif.) containing 2 μg of polyA+RNA per lane were probed using standard techniques (Chirgwin et al. (1979) Biochemistry 18:5294) with a ³²P-labeled DNA fragment encoding the 5′ end of human TANGO 130. This Northern analysis revealed that an approximately 7 kb TANGO 130 mRNA is expressed in heart, brain, placenta, lung, liver, skeletal muscle, pancreas, kidney, spleen, thymus, prostate, testis (highest expression), and uterus. It was not detected in colon, peripheral blood lymphocytes, or small intestine.

In addition, Northern analysis was used to examine TANGO 130 expression in human cancer tissues as follows. Northern blots (Human Multiple Tissue Northern Blots, catalog number 7757-1, Clontech, Palo Alto, Calif.) containing 2 μg of polyA+ RNA per lane were probed using standard techniques (Chirgwin et al. (1979) Biochemistry 18:5294) with a ³²P-labeled DNA fragment encoding the 5′ end of human TANGO 130. This Northern analysis revealed that an approximately 7 kb TANGO 130 mRNA is expressed promyelocytic leukemia cells (HL-60), cervical adenocarcinoma cells (HeLa), chronic myelogenous leukemia cells (K562), lymphoblastic leukemia cells (MOLT-4), colorectal adenocarcinoma cells (SW480), and melanoma cells (G361).

### Example 3

### Characterization of TANGO 130 Proteins

In this example, the predicted amino acid sequences of mouse and human TANGO 130 proteins were compared to amino acid sequences of known proteins and various motifs were identified. In addition, the molecular weights of the human TANGO 130 proteins were predicted.

Mouse TANGO 130 (FIGS. 1A-1D; SEQ ID NO:1) isolated as described above encodes a 714 amino acid protein (FIGS. 1A-1D; SEQ ID NO:3). The signal peptide prediction program SIGNALP Optimized Tool (Nielsen et al. (1997) Protein Engineering 10:1) predicted that mouse TANGO 130 includes a 24 amino acid signal peptide (amino acid 1 to about amino acid 24 of SEQ ID NO:3; SEQ ID NO:5) preceding the 690 amino acid mature protein (about amino acid 25 to amino acid 714 of SEQ ID NO:9; SEQ ID NO:10). A hydropathy plot (Protean™; DNASTAR Inc., Madison, Wis.) of mouse TANGO 130 is shown in FIG. 2. This plot shows the location of the predicted signal peptide (“sp”), the location of cysteines (“cys”) and the MIA homology domain (“MIA”).

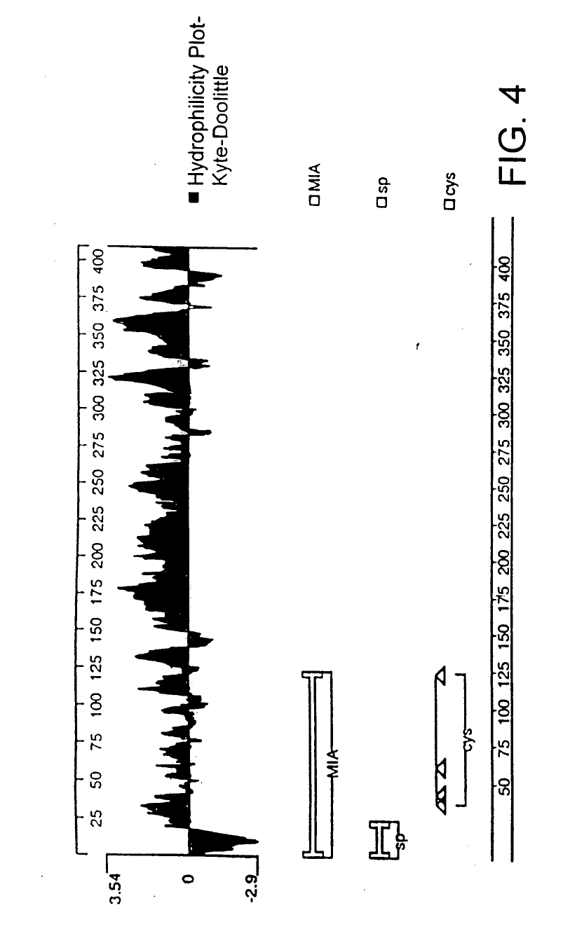

Partial human TANGO 130 cDNA (FIGS. 3A-3B; SEQ ID NO:7) isolated as described above encodes a 410 amino acid protein (FIGS. 3A-3B; SEQ ID NO:9). Alignment with the mouse TANGO 130 nucleotide and amino acid sequences suggests that. the human TANGO 130 molecule is a partial clone. The signal peptide prediction program SIGNALP Optimized Tool (Nielsen et al. (1997) Protein Engineering 10:1) predicted that human TANGO 130 includes a 23 amino acid signal peptide (amino acid 1 to about amino acid 23 of SEQ ID NO:9; SEQ ID NO:11) preceding the 387 amino acid mature protein (about amino acid 24 to amino acid 410 of SEQ ID NO:9; SEQ ID NO:10). A hydropathy plot (Protean™; DNASTAR) of human TANGO 130 is presented in FIG. 4. This plot shows the location of the predicted signal peptide (“sp”), the location of cysteines (“cys”) and the MIA homology domain (“MIA”).

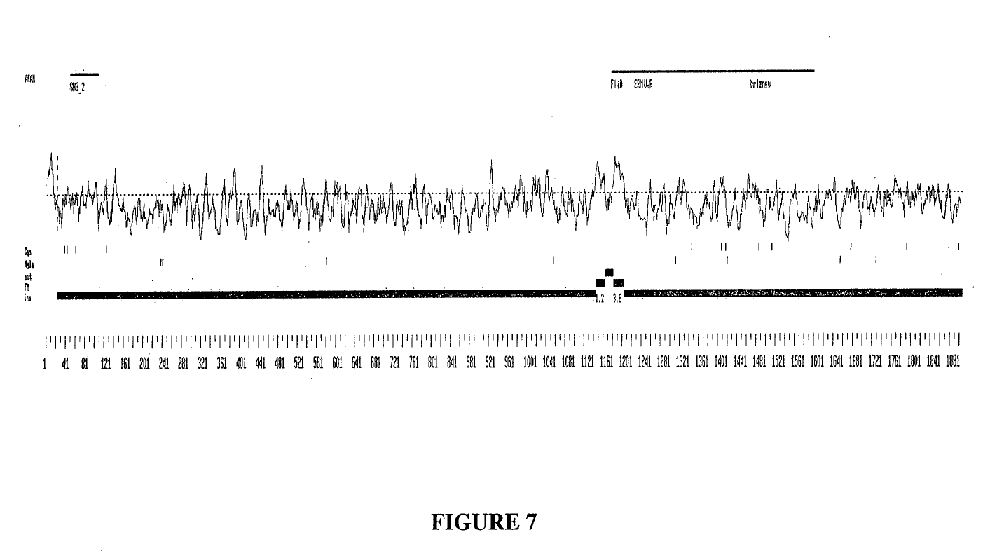

Full length human TANGO 130 cDNA (FIGS. 6A-6J; SEQ ID NO:14) isolated as described above encodes a 1907 amino acid protein (FIGS. 6A-6J; SEQ ID NO:16). The signal peptide prediction program SIGNALP Optimized Tool (Nielsen et al. (1997) Protein Engineering 10:1) predicted that human TANGO 130 includes a 23 amino acid signal peptide (amino acid 1 to about amino acid 23 of SEQ ID NO:16; SEQ ID NO:18) preceding the 1884 amino acid mature protein (about amino acid 24 to amino acid 1907 of SEQ ID NO:16; SEQ ID NO:17). A hydropathy plot (Protean™; DNASTAR) of full length human TANGO 130 is presented in FIG. 7.

The program MegAlign (DNASTAR) was used to align human and mouse TANGO 130 with the MIA/CD-RAP molecules from several species. The “Clustal Method” was used to generate this alignment, with the Gap Penalty set at 5, the Gap Length Penalty set at 10, and the Pairwise Alignment Parameters set as follows: Ktuple=1, Gap Penalty=3, Window=5, and Diagonals Saved=5. As shown in FIG. 5, mouse and human TANGO 130 have a region (amino acids 1-125 of SEQ ID NO:3 and SEQ ID NO:9; SEQ ID NO:6 and SEQ ID NO:12, respectively) of strong homology to MIA/CD-RAP. This region has been named the MIA homology domain. The MIA homology domain contains the following consensus sequence:

(SEQ ID NO:13)

M(X)₆L(X)₄₋₅L(X)₁₉₋₂₁K(L/V)C(A/G)DXECS(X)₇ALXD(X)₃
PDCRF(X)₅GXXVYVXXKL(X)₇WXGSV(X)₄₋₁₂GYFP(X)₁₉DXXDFX
CX,

wherein “M” corresponds to the TANGO 130 initiation methionine and “X” represents any amino acid. The consensus sequence also contains 4 conserved cysteines (underlined). The positions of other highly conserved amino acids are indicated with the single letter amino acid code.

Mature mouse TANGO 130 has a predicted MW of 75.9 kDa (78.5 kDa for immature mouse TANGO 130), not including post-translational modifications. Mature partial human TANGO 130 has a predicted MW of 44.0 kDa (46.5 kDa for immature partial human TANGO 130). Full length human TANGO 130 has a predicted MW of 211.2 kDa (213.7 kDa for immature full length human TANGO 130).

### Example 4

### Preparation of TANGO 130 Fusion Proteins

Recombinant TANGO 130 can be produced in a variety of expression systems. For example, the mature TANGO 130 peptide can be expressed as a recombinant glutathione-S-transferase (GST) fusion protein in E. coli and the fusion protein can be isolated and characterized. Specifically, as described above, TANGO 130 can be fused to GST and this fusion protein can be expressed in E. coli strain PEB 199. Expression of the GST-TANGO 130 fusion protein in PEB 199 can be induced with IPTG. The recombinant fusion protein can be purified from crude bacterial lysates of the induced PEB199 strain by affinity chromatography on glutathione beads.

### Example 5

### Effects of TANGO 130 on Tumor Cells

Cells that can be used to examine effects of TANGO 130 on tumor cell proliferation and tumorigenicity, as described in the following examples, include the human melanoma cell lines WM-266-4 (ATCC Accession Number CRL 1676), G-361 (ATCC Accession Number CRL 1424), and SK-MEL-3 (ATCC Accession Number HTB 69). Other cells include the human astrocytoma glioblastoma line U-373 MG (ATCC Accession Number HTB 17). Additionally, cells, e.g., NIH-3T3 cells (ATCC Accession Number CRL 6442), may be transformed by, e.g., transfection with, e.g., the polyoma virus middle T antigen; by treatment with transforming agents, e.g., phorbol esters; or by mutation of genes involved in tumor suppression, e.g. mutation of p53.

Measurement of TANGO 130 Effect on Cell Proliferation. Tumor cell proliferation following mitogenic stimulation can be measured as follows. ³H-thymidine incorporation is measured after 100 μl tumor cell cultures are pulsed for 6 hours with 0.5 μCi ³H-thymidine (Amersham Corp., Arlington Heights, Ill.). Viable cell numbers are determined by trypan blue exclusion or by staining cultures with propidium iodide (PI), adding a known number of FACS calibration beads (Flow Cytometry Standards Corp., Research Triangle Park, N.C.) and analyzing the samples on a FACScan (Becton Dickinson Immunocytometry Systems, San Jose, Calif.). Beads and viable (PI negative) cells are distinguished by their different forward and side light scattering properties and the ratio of the two is used to calculate the concentration of live cells in the cultures.

Cells undergoing DNA synthesis are identified by addition of ³H-thymidine or BrdU (Boehringer Mannheim Biochemicals, Indianapolis, Ind.) to culture medium. Tumor cells are transfected with 10 μg of plasmid DNAs. After 24 hours, cells are harvested with Trypsin-EDTA solution (Irvine Scientific, Santa Ana, Calif.), seeded into 24-well plates, and either 10 μCi/ml ³H-thymidine or 10 μM BrdU is added. Cultures are continued for 24 hours to increase the yield of labeled cells compared with that obtained with shorter pulse-labelings. The ³H-thymidine-labeled cells are harvested by rinsing cell monolayers with ice-cold phosphate-buffered saline (PBS) and applying 1 ml of 10% TCA. Precipitated DNA is recovered by centrifugation at 16,000×g for 10 minutes, washed with 5% TCA three times and solubilized in 0.5 ml of 0.5 M NaOH. After neutralization by addition of 0.25 ml of 0.1 N HCl, DNA incorporated radioactivity is determined by scintillation counting. BrdU-labeled cells are fixed in 70% ethanol containing 20 mM glycine (pH 2.0) for 20 minutes at −20° C. Incorporated BrdU is detected by an immunofluorescence method involving incubation for 30 minutes with 6 μg/ml of a mouse anti-BrdU primary antibody (Boehringer Mannheim Biochemicals), followed by 1:50 (v/v) of FITC-labeled goat anti-mouse immunoglobulin (DAKO Corp., Carpinteria, Calif.). The proportion of positive nuclei is assessed based on analysis of at least 1000 cells. Incorporation of ³H-thymidine or BrdU reflects the level of cell proliferation in response to treatment of cells with TANGO 130 molecules.

Soft Agar Assays to Measure TANGO 130 Tumor Formation. Growth of cells in soft agar is a commonly used in vitro assay for predicting malignant tumorigenicity in vivo. Soft agar assays can be performed as follows. Vector control and TANGO 130 transfected cells are plated in soft agar and scored for growth after 4 weeks. A 3% solution of agar (at 56° C.) is diluted to a final concentration of 0.6% with growth medium (at 56° C.), pipetted into tissue culture dishes and allowed to solidify at room temperature for 20-30 minutes. At this time, approximately 2×10⁵ cells in a volume of 50 μl are mixed with 0.3% agar (diluted with growth medium at 40° C.), pipetted gently onto the bottom agar layer and allowed to solidify for 20-25 minutes at room temperature. Once solidified, the plates are incubated at 37° C. in a 5% CO₂ atmosphere. Fresh top agar is added once a week. After 4 weeks the plates are stained with neutral red. Stained “colonies” or “foci” of greater than, e.g., eight cells, are counted in TANGO 130 tranfected cells and compared to vector-only transfected or non-transfected cells. The appearance of colonies or foci in soft agar demonstrates growth that is anchorage independent, a hallmark of cellular transformation.

Effects of TANGO 130 on Tumor Cell Tumorigenicity. The following experiments can be done to determine the tumorigenicity of TANGO 130 transfected cells in vivo. To prepare control and TANGO 130 transfected tumor cells for inoculation, cells in exponential growth phase are harvested by brief exposure to 0.25% trypsin+0.2% EDTA solution (w/v). The cell suspension is pipetted to produce a single-cell suspension. The cells are washed and resuspended in Ca²⁺— and Mg²⁺-free HBSS to the desired cell concentration. Cell viability is determined by Trypan-Blue exclusion, and only single-cell suspensions of 90% viability are used. Tumor cells in 0.2 ml HBSS are injected s.c. over the right scapular region. Growth of s.c. tumors is monitored by examination of the mice every day and weekly measurement of tumors with calipers. The mice are sacrificed 2 months after injection, and tumors are processed for hematoxylin and eosin staining.

To measure the effect of TANGO 130 on metastatic potential, an experimental lung metastasis assay can be performed as follows. Approximately 1×10⁶ control or TANGO 130 transfected tumor cells in 0.2 ml of HBSS are injected i.v. into the lateral tail vein of BALB/c nude mice. The mice are killed after 60 days, and tissues (e.g., the lungs) are removed, washed in water, and fixed with Bouin's solution for 24 hours to facilitate counting of tumor nodules as described previously (Radinsky et al. (1994) Oncogene 9:1887; Huang et al. (1996) Oncogene 13:2339). The number of surface tumor nodules is counted under a dissecting microscope. Sections of tissues are stained with hemotoxylin and eosin to confirm that the nodules are melanoma and to identify micrometastasis.

### Example 6

### Effect of TANGO 130 on Melanoma Cell Proliferation

The effect of TANGO 130 on the proliferation of B16 melanoma cells was studied using a recombinant Fc-TANGO 130 fusion protein and a flag tagged TANGO 130 fusion protein. The B16 cells were seeded at 12,000/well in DMEM containing 10% FCS/1× GPS in 10% CO₂ and incubated for 24 hours. They were then re-fed with the same medium containing 0.5% FCS. Next, Fc-TANGO 130 fusion protein or flag tagged TANGO 130 fusion protein was added to the cell culture and the cultures were incubated for 6 days. At the end of treatment, the cells were trypsinized and quantified using a Coulter counter.

Flag tagged TANGO 130 fusion protein increased B16 cell proliferation by 42% when administered at 4 μg/ml. In contrast, Fc-TANGO 130 fusion protein inhibited B16 cell proliferation by 18% when administered at 4 μg/ml.

### Example 7

### In situ Expression Analysis of Murine TANGO 130

A probe encoding the MIA domain of murine TANGO 130 was used to perform in situ expression analysis of murine TANGO 130. This analysis revealed that murine TANGO 130 expression is highest in liver, testes, ovary, and the submandibular gland. The fact that expression was observed in liver and testes is consistent with the results of Northern blot analysis (Example 2). However, the in situ expression analysis did not detect TANGO 130 expression in the heart while the Northern blot analysis revealed high level TANGO 130 expression in the heart. Overall, the in situ expression analysis revealed expression of TANGO 130 in the following adult murine tissues: brain (signal slightly above background, most noticeable in the olfactory bulb), eye and harderian gland (signal observed in the retina), submandibular gland (strong, ubiquitous signal), stomach (signal observed in the mucosal epithelium), liver (strong, ubiquitous signal), kidney (signal, slightly above background), adrenal gland (signal slightly above background and slightly stronger signal in the medulla), colon (signal above background in the muscle layer), small intestine (signal above background), thymus (ubiquitous signal above background), lymph node (ubiquitous signal above background), testes (strong signal that outlines the seminiferous vesicles), ovaries (strong multifocal signal), placenta (ubiquitous signal that is stronger in the decidua region). No expression was detected in the following tissues: spinal cord, white fat, brown fat, heart, lung, spleen, pancreas, skeletal muscle, and bladder.

### Example 8

### Identification of Tissues having TANGO 130 Binding Sites

Two different alkaline phosphatase-murine TANGO 130 fusion proteins were used to identify tissues having TANGO 130 binding sites. One fusion protein consisted of alkaline phosphatase fused to the N-terminus of murine TANGO 130 (AP-mT130) and the other fusion protein consisted of alkaline phosphatase fused to the C-terminus of murine TANGO 130 (mT130-AP). The screening of tissue sections with the fusion proteins was performed essentially as described previously (Cheng and Flanagan (1994) Cell 79:157-168). Briefly, fresh frozen tissue sections (8 μm) were prepared and rinsed in HBHA (Hank's balanced salt solution supplemented with 20 M Hepes, pH 7, 0.05% BSA and 0.1% sodium azide). The tissue sections were then incubated for 1 h at RT with supernatant containing AP-mT130, mT130-AP or alkaline phosphatase (AP) at a concentration of 5 nM. After incubation, the tissue sections were washed six times in HBHA, fixed in a solution containing 60% acetone, 3% formaldehyde and 20 mM Hepes, pH 7.5, washed three times in HBS (20 mM Hepes, pH 7.5, 150 mM NaCl) and then heated for 30 min at 65° C. to inactivate endogenous alkaline phosphatase activity. Bound AP and AP fusion protein was detected by developing sections in BCIP/NBT substrate solution (100 mM Tris-HCl, pH 9.5, 100 mM NaCl, 5 mM MgCl, 0.17 mg/ml BCIP and 0.33 mg/ml NBT).

Cell supernatant containing AP-mT130 or mT130-AP was used to screen tissue sections of embryos (day 14.5 of prenatal development), whole mice (postnatal day 1.5) and a collection of adult mouse tissues for T130 binding sites. Both AP-mT130 and mT130-AP, but not AP itself, bound to connective tissue, cartilage and bone. Binding of AP-mT130 and mT130-AP was also observed in the following adult mouse tissues: esophagus (connective tissue), trachea (cartilage, connective tissue, and chondrocytes), aorta (connective tissue), eye (cornea/sclera), adrenal gland (capsule), spleen (capsule, connective tissue, and scattered cells throughout red pulp), skeletal muscle (connective tissue), skin (derma and connective tissue), testes (tunica albuginea), bladder (connective tissue), uterus/ovaries (connective tissue), kidney (connective tissue), and brain (meninges).

AP-mT130 and mT130-AP were also found to bind to the mouse fibroblast cell line 3T3L1, the human chondrosarcoma cell line HTB-94, and human and mouse osteoblasts.

Binding of AP-mT130 and mT130-AP to tissues and cells was completely blocked in the presence of 100-fold excess of purified Fc-TANGO 130 fusion protein, but not by a control Fc fusion protein indicating that the binding is specific for murine TANGO 130.

In addition, AP-mT130 and mT130-AP were found to bind to connective tissue layers in human ovaries, testes, breast, and adipose tissue. This result demonstrates that murine TANGO 130 is able to bind to human tissues.

### Example 9

### TANGO 130 is Secreted

A secretion assay revealed that alkaline phosphatase TANGO 130 MIA domain fusion protein and flag tagged TANGO 130 MIA domain fusion protein are secreted from 293T cells. Briefly, 8×10⁵ 293T cells were plated per well in a 6-well plate and the cells were incubated in growth medium (DMEM, 10% fetal bovine serum, penicillin/strepomycin) at 37° C., 5% CO₂ overnight. The 293T cells were transfected with a vector expressing a TANGO 130 fusion protein and 10 ìg LipofectAMINE (GIBCO/BRL Cat. # 18324-012)/well according to the protocol for GIBCO/BRL LipofectAMINE. The transfectant was removed 5 hours later and fresh growth medium was added to allow the cells to recover overnight. The medium was removed and each well was gently washed twice with DMEM without methionine and cysteine (ICN Cat. # 16-424-54). Next, 1 ml DMEM without methionine and cysteine with 50 ìCi Trans-35S (ICN Cat. # 51006) was added to each well and the cells were incubated at 37° C., 5% CO₂ for the appropriate time period. A 150 ìl aliquot of conditioned medium was obtained and 150 ìl of 2×SDS sample buffer was added to the aliquot. The sample was heat-inactivated and loaded on a 4-20% SDS-PAGE gel. The gel was fixed and the presence of secreted protein was detected by autoradiography.

### Example 10

### Preparation of Anti-TANGO 130 Antibodies

The following peptides were used to prepare polyclonal antibodies in rabbits: DLSHGRRFSDLK (amino acids 25 to 36 of murine TANGO 130; SEQ ID NO:20); EDFTGPDCRFVNFKK (amino acids 54 to 68 of murine TANGO 130; SEQ ID NO:21); QLDPSTGRRFSEHK (amino acids 23 to 36 of human TANGO 130; SEQ ID NO:22); EDFTGPDCRFVNFKK (amino acids 54 to 68 of human TANGO 130; SEQ ID NO:23); and GFLELYNSAATDSE (amino acids 142 to 155 of human TANGO 130; SEQ ID NO:24). Each polyclonal antibody was affinity purified using the corresponding. These polyclonal antibodies are able to bind various TANGO 130 fusion proteins.

### Example 11

### Effect of TANGO 130 on Embryonic Development

The effect of TANGO 130 on embryonic development was investigated by injecting Xenopus embryos with murine TANGO 130 MIA domain (T130(MIA)) mRNA or flag tagged murine TANGO 130 MIA domain fusion protein (T130(MIA)-flag) mRNA. Briefly, capped mRNAs were synthesized using SP6 RNA polymerase and the using mMESSAGE mMACHINE kit (Ambion, Austin, Tex.) according to the manufacturer's instructions. Linearized plasmids encoding T130(MIA) or T130(MIA)-flag were used as templates for mRNA synthesis. In vitro transcribed capped RNA was purified using RNAesy kit (Qiagen) and analyzed by gel electrophoresis.

Xenopus embryos were obtained by in vitro fertilization, dejellied in 2% cysteine HCl (pH 7.6), washed thoroughly in Modified Ringers solution, and incubated at 15-25° C. Embryos were transferred to injection solution (Modified Ringers solution containing 3% Ficoll) prior to injections. Next, 1 ng and 2.5 ng of T130(MIA) mRNA and T130(MIA)-flag mRNA was injected into one blastomere at the 2-cell stage. Embryos were transferred to 0.1× MMR from the injection solution after approximately 6 hours and grown until the appropriate stage.

Examination two days later of embryos injected with TANGO 130 mRNAs showed an overexpression phenotype (slight to moderate enlargement of head and anterior trunk region). These results suggest that TANGO 130 has an effect on early tissue development/differentiation. Embryos for histological examination were fixed in 4% formaldehyde overnight, embedded in paraffin and stained by standard procedures.

In another study, 2 ng of T130(MIA) mRNA was injected into the animal pole of each of the 2 blastomeres at the 2-cell stage. Animal caps from uninjected or injected embryos were explanted at stage 9 and cultured in 1× Modified Ringers containing 0.01% BSA and 50 ug/ml gentamycin. Animal caps were cultured until control embryos have reached stage 23-24. Animal cap tissue was lysed and total RNA was extracted using RNeasy kit (Qiagen). Next, RT-PCR was performed on these samples using gene-specific primers and appropriate annealing temperatures and the products were analyzed by gel electrophoresis. The RT-PCR analysis indicated weak induction of Sox-17, an endodermal specific marker.

### Example 12

### Chromosomal Mapping of Murine TANGO 130

Murine TANGO 130 was mapped to chromosome 5 between markers D5Mit195 and D5Mit15. The following genes are located in this region: gprk21 (a G protein coupled receptor, kinase), add1 (adducin), lx (luxate), drd5 (dopamine receptor 5), bp3 (alloantigen), qdpr (quininoid dihyropteridine reductase), sod3 (superoxide dismutase3), cckr (cholecystokinin A receptor), pgm3 (phosphoglucomutase), arp (lymphoid, erythroid hyperplasia), gckr (glucokinase regulatory protein), hdh (Huntington's homolog), and khk (ketohexokinase).

The region to which murine TANGO 130 maps corresponds to human 7q, 7p, 18p1, 4p1, 14q.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule selected from the group consisting of:
a) a nucleic acid molecule comprising a nucleotide sequence which is at least 80% identical to the nucleotide sequence of SEQ ID NO:1, SEQ ID NO:2, SEQ ID NO:7, SEQ ID NO:8, the cDNA insert of the plasmid deposited with ATCC as Accession Number 98823, the cDNA insert of the plasmid deposited with ATCC as Accession Number 98844, the cDNA insert of the plasmid deposited with ATCC as Accession Number 98845, or a complement thereof;
b) a nucleic acid molecule comprising a fragment of at least 500 nucleotides of the nucleotide sequence of SEQ ID NO:1, SEQ ID NO:2, SEQ ID NO:7, SEQ ID NO:8, the cDNA insert of the plasmid deposited with ATCC as Accession Number 98823, the cDNA insert of the plasmid deposited with ATCC as Accession Number 98844, the cDNA insert of the plasmid deposited with ATCC as Accession Number 98845, or a complement thereof;
c) a nucleic acid molecule comprising a fragment of at least 5820 nucleotides of the nucleotide sequence of SEQ ID NO:14, or a complement thereof;
d) a nucleic acid molecule comprising a fragment of at least 3610 nucleotides of the nucleotide sequence of SEQ ID NO:15, or a complement thereof;
e) a nucleic acid molecule which encodes a polypeptide comprising the amino acid sequence of SEQ ID NO:3, SEQ ID NO:4, SEQ ID NO:9, SEQ ID NO:10, SEQ ID NO:16, SEQ ID NO:17, an amino acid sequence encoded by the cDNA insert of the plasmid deposited with ATCC as Accession Number 98823, an amino acid sequence encoded by the cDNA insert of the plasmid deposited with ATCC as Accession Number 98844, or an amino acid sequence encoded by the cDNA insert of the plasmid deposited with ATCC as Accession Number 98845;
f) a nucleic acid molecule which encodes a fragment of a polypeptide comprising the amino acid sequence of SEQ ID NO:3, SEQ ID NO:4, SEQ ID NO:9, or SEQ ID NO:10, wherein the fragment comprises at least 15 contiguous amino acids of SEQ ID NO:3, SEQ ID NO:4, SEQ ID NO:9, or SEQ ID NO:10, the polypeptide encoded by the cDNA insert of the plasmid deposited with ATCC as Accession Number 98823, the polypeptide encoded by the cDNA insert of the plasmid deposited with ATCC as Accession Number 98844, or the polypeptide encoded by the cDNA insert of the plasmid deposited with ATCC as Accession Number 98845;
g) a nucleic acid molecule which encodes a fragment of a polypeptide comprising the amino acid sequence of SEQ ID NO:16 or SEQ ID NO:17, wherein the fragment comprises at least 1200 contiguous amino acids of SEQ ID NO:16 or SEQ ID NO:17; and
h) a nucleic acid molecule which encodes a naturally occurring allelic variant of a polypeptide comprising the amino acid sequence of SEQ ID NO:3, SEQ ID NO:4, SEQ ID NO:9, SEQ ID NO:10, SEQ ID NO:16, SEQ ID NO:17, an amino acid sequence encoded by the cDNA insert of the plasmid deposited with ATCC as Accession Number 98823, an amino acid sequence encoded by the cDNA insert of the plasmid deposited with ATCC as Accession Number 98844, or an amino acid sequence encoded by the cDNA insert of the plasmid deposited with ATCC as Accession Number 98845, wherein the nucleic acid molecule hybridizes to a nucleic acid molecule comprising SEQ ID NO:1, SEQ ID NO:2, SEQ ID NO:7, SEQ ID NO:8, SEQ ID NO:14, SEQ ID NO:15, or a complement thereof under stringent conditions.

2. The isolated nucleic acid molecule of claim 1, which is selected from the group consisting of:
a) a nucleic acid comprising the nucleotide sequence of SEQ ID NO:1, SEQ ID NO:2, SEQ ID NO:7, SEQ ID NO:8, SEQ ID NO:14, SEQ ID NO:15, the cDNA insert of the plasmid deposited with ATCC as Accession Number 98823, the cDNA insert of the plasmid deposited with ATCC as Accession Number 98844, the cDNA insert of the plasmid deposited with ATCC as Accession Number 98845, or a complement thereof; and
b) a nucleic acid molecule which encodes a polypeptide comprising the amino acid sequence of SEQ ID NO:3, SEQ ID NO:4, SEQ ID NO:9, SEQ ID NO:10, SEQ ID NO:16, SEQ ID NO:17, an amino acid sequence encoded by the cDNA insert of the plasmid deposited with ATCC as Accession Number 98823, an amino acid sequence encoded by the cDNA insert of the plasmid deposited with ATCC as Accession Number 98844, or an amino acid sequence encoded by the cDNA insert of the plasmid deposited with ATCC as Accession Number 98845.

3. The nucleic acid molecule of claim 1 further comprising vector nucleic acid sequences.

4. The nucleic acid molecule of claim 1 further comprising nucleic acid sequences encoding a heterologous polypeptide.

5. A host cell which contains the nucleic acid molecule of claim 1.

6. The host cell of claim 5 which is a mammalian host cell.

7. A non-human mammalian host cell containing the nucleic acid molecule of claim 1.

8. An isolated polypeptide selected from the group consisting of:
a) a fragment of a polypeptide comprising the amino acid sequence of SEQ ID NO:3, SEQ ID NO:4, SEQ ID NO:9, or SEQ ID NO:10, wherein the fragment comprises at least 15 contiguous amino acids of SEQ ID NO:3, SEQ ID NO:4, SEQ ID NO:9, or SEQ ID NO:10;
b) a fragment of a polypeptide comprising the amino acid sequence of SEQ ID NO:16 or SEQ ID NO:17, wherein the fragment comprises at least 1200 contiguous amino acids of SEQ ID NO:16 or SEQ ID NO:17;
c) a naturally occurring allelic variant of a polypeptide comprising the amino acid sequence of SEQ ID NO:3, SEQ ID NO:4, SEQ ID NO:9, SEQ ID NO:10, SEQ ID NO:16, SEQ ID NO:17, an amino acid sequence encoded by the cDNA insert of the plasmid deposited with ATCC as Accession Number 98823, an amino acid sequence encoded by the cDNA insert of the plasmid deposited with ATCC as Accession Number 98844, or an amino acid sequence encoded by the cDNA insert of the plasmid deposited with ATCC as Accession Number 98845, wherein the polypeptide is encoded by a nucleic acid molecule which hybridizes to a nucleic acid molecule comprising SEQ ID NO:1, SEQ ID NO:2, SEQ ID NO:7, SEQ ID NO:8, SEQ ID NO:14, SEQ ID NO:15, or a complement thereof, under stringent conditions; and
d) a polypeptide which is encoded by a nucleic acid molecule comprising a nucleotide sequence which is at least 80% identical to a nucleic acid comprising the nucleotide sequence of SEQ ID NO:1, SEQ ID NO:2, SEQ ID NO:7, SEQ ID NO:8, SEQ ID NO:14, SEQ ID NO:15, or a complement thereof.

9. The isolated polypeptide of claim 8 comprising the amino acid sequence of SEQ ID NO:3, SEQ ID NO:4, SEQ ID NO:9, SEQ ID NO:10, SEQ ID NO:16, SEQ ID NO:17, an amino acid sequence encoded by the cDNA insert of the plasmid deposited with ATCC as Accession Number 98823, an amino acid sequence encoded by the cDNA insert of the plasmid deposited with ATCC as Accession Number 98844, or an amino acid sequence encoded by the cDNA insert of the plasmid deposited with ATCC as Accession Number 98845.

10. The polypeptide of claim 8 further comprising heterologous amino acid sequences.

11. An antibody which selectively binds to a polypeptide of claim 8.

12. A method for producing a polypeptide selected from the group consisting of:
a) a polypeptide comprising the amino acid sequence of SEQ ID NO:3, SEQ ID NO:4, SEQ ID NO:9, SEQ ID NO:10, SEQ ID NO:16, SEQ ID NO:17 an amino acid sequence encoded by the cDNA insert of the plasmid deposited with ATCC as Accession Number 98823, or an amino acid sequence encoded by the cDNA insert of the plasmid deposited with ATCC as Accession Number 98844, an amino acid sequence encoded by the cDNA insert of the plasmid deposited with ATCC as Accession Number 98845;
b) a polypeptide comprising a fragment of the amino acid sequence of SEQ ID NO:3, SEQ ID NO:4, SEQ ID NO:9, SEQ ID NO:10, an amino acid sequence encoded by the cDNA insert of the plasmid deposited with ATCC as Accession Number 98823, an amino acid sequence encoded by the cDNA insert of the plasmid deposited with ATCC as Accession Number 98844, or an amino acid sequence encoded by the cDNA insert of the plasmid deposited with ATCC as Accession Number 98845, wherein the fragment comprises at least 15 contiguous amino acids of SEQ ID NO:3, SEQ ID NO:4, SEQ ID NO:9, SEQ ID NO:10, an amino acid sequence encoded by the cDNA insert of the plasmid deposited with ATCC as Accession Number 98823, an amino acid sequence encoded by the cDNA insert of the plasmid deposited with ATCC as Accession Number 98844, or an amino acid sequence encoded by the cDNA insert of the plasmid deposited with ATCC as Accession Number 98845;
c) a polypeptide comprising a fragment of the amino acid sequence of SEQ ID NO:16 or SEQ ID NO:17, wherein the fragment comprises at least 1200 contiguous amino acids of SEQ ID NO:16 or SEQ ID NO:17; and
d) a naturally occurring allelic variant of a polypeptide comprising the amino acid sequence of SEQ ID NO:3, SEQ ID NO:4, SEQ ID NO:9, SEQ ID NO:10, SEQ ID NO:16, SEQ ID NO:17, an amino acid sequence encoded by the cDNA insert of the plasmid deposited with ATCC as Accession Number 98823, an amino acid sequence encoded by the cDNA insert of the plasmid deposited with ATCC as Accession Number 98844, or an amino acid sequence encoded by the cDNA insert of the plasmid deposited with ATCC as Accession Number 98845, wherein the polypeptide is encoded by a nucleic acid molecule which hybridizes to a nucleic acid molecule comprising SEQ ID NO:1, SEQ ID NO:2, SEQ ID NO:7, SEQ ID NO:8, SEQ ID NO:14, SEQ ID NO:15, or a complement thereof, under stringent conditions;
comprising culturing the host cell of claim 5 under conditions in which the nucleic acid molecule is expressed.

13. The method of claim 12, wherein the polypeptide comprises the amino acid sequence of SEQ ID NO:3 or SEQ ID NO:4, SEQ ID NO:9, SEQ ID NO:10, SEQ ID NO:16, SEQ ID NO:17, an amino acid sequence encoded by the cDNA insert of the plasmid deposited with ATCC as Accession Number 98823, an amino acid sequence encoded by the cDNA insert of the plasmid deposited with ATCC as Accession Number 98844, or an amino acid sequence encoded by the cDNA insert of the plasmid deposited with ATCC as Accession Number 98845.

14. A method for detecting the presence of a polypeptide of claim 8 in a sample, comprising:
a) contacting the sample with a compound which selectively binds to a polypeptide of claim 8; and
b) determining whether the compound binds to the polypeptide in the sample.

15. The method of claim 14, wherein the compound which binds to the polypeptide is an antibody.

16. A kit comprising a compound which selectively binds to a polypeptide of claim 8 and instructions for use.

17. A method for detecting the presence of a nucleic acid molecule of claim 1 in a sample, comprising the steps of:
a) contacting the sample with a nucleic acid probe or primer which selectively hybridizes to the nucleic acid molecule; and
b) determining whether the nucleic acid probe or primer binds to a nucleic acid molecule in the sample.

18. The method of claim 17, wherein the sample comprises mRNA molecules and is contacted with a nucleic acid probe.

19. A kit comprising a compound which selectively hybridizes to a nucleic acid molecule of claim 1 and instructions for use.

20. A method for identifying a compound which binds to a polypeptide of claim 8 comprising the steps of:
a) contacting a polypeptide, or a cell expressing a polypeptide of claim 8 with a test compound; and
b) determining whether the polypeptide binds to the test compound.

21. The method of claim 20, wherein the binding of the test compound to the polypeptide is detected by a method selected from the group consisting of:
a) detection of binding by direct detecting of test compound/polypeptide binding;
b) detection of binding using a competition binding assay; and
c) detection of binding using an assay for TANGO 130-mediated signal transduction.

22. A method for modulating the activity of a polypeptide of claim 8 comprising contacting a polypeptide or a cell expressing a polypeptide of claim 8 with a compound which binds to the polypeptide in a sufficient concentration to modulate the activity of the polypeptide.

23. A method for identifying a compound which modulates the activity of a polypeptide of claim 8, comprising:
a) contacting a polypeptide of claim 8 with a test compound; and
b) determining the effect of the test compound on the activity of the polypeptide to thereby identify a compound which modulates the activity of the polypeptide.
